SEQUENCE LISTING (84 sequences)

SEQ ID NO: 1 - DNA, 1258 bp, Homo sapiens
gcctccaaggtttgtcttgaagcatagctccagctggagggtaccttttaagctgttcaaggtcaagatgaatacaaactcaaaggaggttttatccctgggtgttcaagttcccgaggcatgggaagaacttctgacaatgaaagtggaagcaaaaagtcaccttcaatggcaggaatccagactgaaacgcagtaatccactggcaagggaaatcttccgaaggcactttcgacagctgtgctaccaagagacccctggaccaagggaggctcttactcgactccaggaactttgctaccagtggttgaggccacatgtgagcacaaaggagcagattttggatctgctggtgctggagcagtttctatccattctgcccaaggagctccagggctgggtgagggaacactgtccagagagtggagaagaggctgtgattttgctggaggatctggagagagagctcgatgaaccacaacatgagatggtggcccacagacacagacaagaagtcctctgtaaagagatggtgcctctagcagagcagacaccactgacccttcagtcccagcctaaggagccacagctcacatgtgactctgctcagaagtgccattctattggagagacagatgaagtaaccaagactgaggacagagagttggtgctaaggaaagactgtcctaagatagtggaaccacatgggaaaatgtttaatgagcagacctgggaggtatcacagcaggatccctcacatggagaagttggtgaacataaggataggatagagaggcagtggggaaacctcttaggagaggggcaacacaaatgtgatgaatgtgggaagagctttactcagagctcaggtctcattcgacatcaaagaattcatactggagaaagaccttatgaatgtaatgaatgtgggaaagccttcagtcgaagttctggtctttttaatcaccgaggaatccacaatatacagaaacggtaccactgcaaggagtgtgggaaggtcttcagtcagagtgcgggtcttatccagcatcagagaatccacaaaggagaaaagccgtatcagtgcagccagtgcagtaagagctacagtcggcgttcatttctcattgaacatcagagaagccacacaggggagcgacctcaccagtgcattgaatgtgggaaaagctttaatcgacactgcaacctcattcgccatcagaagatccacacagtggctgagctggtctagggcttg

SEQ ID NO: 2 - protein, 394 aa, Homo sapiens
MNTNSKEVLSLGVQVPEAWEELLTMKVEAKSHLQWQESRLKRSNPLAREIFRRHFRQLCYQETPGPREALTRLQELCYQWLRPHVSTKEQILDLLVLEQFLSILPKELQGWVREHCPESGEEAVILLEDLERELDEPQHEMVAHRHRQEVLCKEMVPLAEQTPLTLQSQPKEPQLTCDSAQKCHSIGETDEVTKTEDRELVLRKDCPKIVEPHGKMFNEQTWEVSQQDPSHGEVGEHKDRIERQWGNLLGEGQHKCDECGKSFTQSSGLIRHQRIHTGERPYECNECGKAFSRSSGLFNHRGIHNIQKRYHCKECGKVFSQSAGLIQHQRIHKGEKPYQCSQCSKSYSRRSFLIEHQRSHTGERPHQCIECGKSFNRHCNLIRHQKIHTVAELV

SEQ ID NO: 3 - DNA, 16 bp, Artificial Sequence
  anchored primer for PIG12
aagcttttttttttta

SEQ ID NO: 4 - DNA, 13 bp, Artificial Sequence
  H-AP21 primer for PIG12
aagctttctctgg

SEQ ID NO: 5 - DNA, 2403 bp, Homo sapiens
cccagcagttataacagcagtgggcagttgatggcaagtaaaatggatacctgctctagtaacctgaataacagcatatacaaaaagctgttaacttaggaaaagggactgctgggaggttaaaaagaaaagtttataaaagtgaataacctgaggattctattagtccccacccaaactttattgattcacctcctaaaacaacagatgtacgacttgcatacctgctttttatgggagctgtcaagcatgtatttttgtcaattaccagaaagataacaggacgagatgacggtgttattccaagggaatattgccaatgctacagtaataaatgaatgtcacttctggatatagctaggtgacatatacatacttacatgtgtgtatatgtagatgtatgcacacacatatattatttgcagtgcagtatagaataggcactttaaaacactctttccccgcaccccagcaattatgaaaataatctctgattccctgatttaatatgcaaagtctaggttggtagagtttagccctgaacatttcatggtgttcatcaacagtgagagactccatagtttgggcttgtaccacttttgcaaataagtgtattttgaaattgtttgacggcaaggtttaagttattaagaggtaagacttagtactatctgtgcgtagaagttctagtgttttcaattttaaacatatccaagtttgaattcctaaaattatggaaacagatgaaaagcctctgttttgatatgggtagtattttttacattttacacactgtacacataagccaaaactgagcataagtcctctagtgaatgtaggctggctttcagagtaggctattcctgagagctgcatgtgtccgcccccgatggaggactccaggcagcagacacatgccagggccatgtcagacacagattggccagaaaccttcctgctgagcctcacagcagtgagactggggccactacatttgctccatcctcctgggattggctgtgaactgatcatgtttatgagaaactggcaaagcagaatgtgatatcctaggaggtaatgaccatgaaagacttctctacccatcttaaaaacaacgaaagaaggcatggacttctggatgcccatccactgggtgtaaacacatctagtagttgttctgaaatgtcagttctgatatggaagcacccattatgcgctgtggccactccaataggtgctgagtgtacagagtggaataagacagagacctgccctcaagagcaaagtagatcatgcatagagtgtgatgtatgtgtaataaatatgtttcacacaaacaaggcctgtcagctaaagaagtttgaacatttgggttactatttcttgtggttataacttaatgaaaacaatgcagtacaggacatatattttttaaaataagtctgatttaattgggcactatttatttacaaatgttttgctcaatagattgctcaaatcaggttttcttttaagaatcaatcatgtcagtctgcttagaaataacagaagaaaatagaattgacattgaaatctaggaaaattattctataatttccatttacttaagacttaatgagactttaaaagcattttttaacctcctaagtatcaagtatagaaaatcttcatggaattcacaaagtaatttggaaattaggttgaaacatatctcttatcttacgaaaaaatggtagcattttaaacaaaatagaaagttgcaaggcaaatgtttatttaaaagagcaggccaggcgcggtggctcacgcctgtaatcccagcactttgggaggctgaggcgggtggatcacgaggtcaggagatcgagaccatcctggctaacacggtgaaacccgtctctactaaaaatgcaaaaaaaattagccgggcgtggtggcaggcacctgtagtcccagctactcgggaggctgaggcaggagactggcgtgaacccaggaggcggaccttgtagtgagccgagatcgcgccactgtgctccagcctgggcaacagagcaagactccatctcaaaaaataaaaataaataaaaaataaaaaaataaaagagcaaactatttccaaataccatagaataacttacataaaagtaatataactgtattgtaagtagaagctagcactggttttattaatttagtgactattcattttatctaaatcagtgaagatttactgtcattgtttattagtctgtatatattaaaatatgatatcattaatgtacttacaaaatagtatgtcactgtttttatgttcattcttaaaaacataacctgtattaataaatgtgaacatttgcttggtaaaaaaaaaaaaaaaaaaaaaaaaaaaaaaaaaaaaaa

SEQ ID NO: 6 - protein, 167 aa, Homo sapiens
MCPPPMEDSRQQTHARAMSDTDWPETFLLSLTAVRLGPLHLLHPPGIGCELIMFMRNWQSRMMTMKDFSTHLKNNERRHGLLDAHPLGVNTSSSCSEMSVLIWKHPLCAVATPIGAECTEWNKTETCPQEQSRSCIECDVCVINMFHTNKACQLKKFEHLGYYFLWL

SEQ ID NO: 7 - DNA, 16 bp, Artificial Sequence
  anchored primer for PIG18
aagctttttttttttc

SEQ ID NO: 8 - DNA, 13 bp, Artificial Sequence
  H-AP11 primer for PIG18
aagcttcgggtaa

SEQ ID NO: 9 - DNA, 2150 bp, Homo sapiens
gcggacagtgcggaactaaagcaaatggttatgagccttagagtttctgaactccaagtactgttgggctacgccgggagaaacaagcacggacgcaaacacgaacttctcacaaaagccctgcatttgctaaaggctggctgtagtcctgctgtgcaaatgaaaattaaggaactctataggcggcggttcccacagaaaatcatgacgcctgcagacttgtccatccccaacgtacattcaagtcctatgccagcaactttgtctccatctaccattccacaactcacttacgatggtcaccctgcatcatcgccattactccctgtttctcttctgggacctaaacatgaactggaactcccacatcttacatcagctcttcacccagtccatccggatataaaacttcaaaaattaccattttatgatttactggatgaactgataaaacccaccagtctagcatcagacaacagtcagcgctttcgagaaacctgttttgcatttgccttgacaccacaacaagtgcagcaaatcagtagttccatggatatttctgggaccaaatgtgacttcacagtacaggtccagttaaggttttgtttatcagaaaccagttgtccacaagaagatcacttcccacccaatctttgtgtgaaagtgaatacaaaaccttgcagccttccaggttaccttccacctacaaaaaatggcgtggaaccaaagcgacccagccgaccaattaatatcacctcacttgtccgactgtccacaacagtaccaaacacgattgttgtttcttggactgcagaaattggaagaaactattccatggcagtatatcttgtaaaacagttgtcctcaacagttcttcttcagaggttacgagcaaagggaataaggaatccggatcattctagagctttaattaaagagaagttgactgcggatccagacagtgaaatagctacaaccagcctaagggtttctctactatgtccacttggtaaaatgcggctgacaattccgtgtcgggcccttacatgttctcatctacaatgttttgacgcaactctttacattcagatgaatgagaaaaaaccaacctgggtttgtcctgtctgtgataagaaggctccatatgaacaccttattattgatggcttgtttatggaaatcctaaagtactgtacagactgtgatgaaatacaatttaaggaggatggcacttgggcaccgatgagatcaaaaaaggaagtacaggaagtttctgcctcttacaatggagtcgatggatgcttgagctccacattggagcatcaggtagcgtctcaccaccagtcctcaaataaaaacaagaaagtagaagtgattgacctaaccatagacagttcatctgatgaagaggaagaagagccatctgccaagaggacctgtccttccctatctcccacatcaccactaaataataaaggcattttaagtcttccacatcaagcatctccagtatcccgcaccccaagccttcctgctgtagacacaagctacattaatacctccctcatccaagactataggcatcctttccacatgacacccatgccttacgacttacaaggattagatttctttcctttcttatcaggagacaatcagcattacaacacctccttgcttgccgctgcagcagcagcagtttcagatgatcaagacctcctacactcgtctcggtttttcccgtatacctcctcacagatgtttcttgatcagttaagtgcaggaggcagtacttctctgccaaccaccaatggaagcagtagtggcagtaacagcagcctggtttcttccaacagcctaagggaaagccatagccacaccgtcacaaacaggagcagcacggacacggcatccatctttggcatcataccagacattatttcattggactgattcccaggccctgctgctcccatccccaccccagatcgaatgaacttggcagaaagaagagaactttgtgctctgttttaccttactctgtttagaaaagtatacaagcgtgttttttttcctttttttagggaaaaaattaaaagaaatgtacagagaaaaaaataaaaaaaaaataaaaaaaaaaaaaaaaaaaa

SEQ ID NO: 10 - protein, 642 aa, Homo sapiens
MVMSLRVSELQVLLGYAGRNKHGRKHELLTKALHLLKAGCSPAVQMKIKELYRRRFPQKIMTPADLSIPNVHSSPMPATLSPSTIPQLTYDGHPASSPLLPVSLLGPKHELELPHLTSALHPVHPDIKLQKLPFYDLLDELIKPTSLASDNSQRFRETCFAFALTPQQVQQISSSMDISGTKCDFTVQVQLRFCLSETSCPQEDHFPPNLCVKVNTKPCSLPGYLPPTKNGVEPKRPSRPINITSLVRLSTTVPNTIVVSWTAEIGRNYSMAVYLVKQLSSTVLLQRLRAKGIRNPDHSRALIKEKLTADPDSEIATTSLRVSLLCPLGKMRLTIPCRALTCSHLQCFDATLYIQMNEKKPTWVCPVCDKKAPYEHLIIDGLFMEILKYCTDCDEIQFKEDGTWAPMRSKKEVQEVSASYNGVDGCLSSTLEHQVASHHQSSNKNKKVEVIDLTIDSSSDEEEEEPSAKRTCPSLSPTSPLNNKGILSLPHQASPVSRTPSLPAVDTSYINTSLIQDYRHPFHMTPMPYDLQGLDFFPFLSGDNQHYNTSLLAAAAAAVSDDQDLLHSSRFFPYTSSQMFLDQLSAGGSTSLPTTNGSSSGSNSSLVSSNSLRESHSHTVTNRSSTDTASIFGIIPDIISLD

SEQ ID NO: 11 - DNA, 16 bp, Artificial Sequence
  anchored primer for PIG23
aagctttttttttttc

SEQ ID NO: 12 - DNA, 13 bp, Artificial Sequence
  H-AP33 primer for PIG23
aagcttgctgctc

SEQ ID NO: 13 - DNA, 446 bp, Homo sapiens
tggtcacagtggaaggatcatgggagaaacagaagggaagaaagatgaggctgattataagcgactgcagaccttccctctggtcaggcactcggacatgccagaggagatgcgcgtggagaccatggagctatgtgtcacagcctgtgagaaattctccaacaacaacgagagcgccgccaagatgatcaaagagacaatggacaagaagttcggctcctcctggcacgtggtgatcggcgagggctttgggtttgagatcacccacgaggtgaagaacctcctctacctgtacttcgggggcaccctggctgtgtgcgtctggaagtgctcctgacactctgcccctatttaaaagcctgttgattcaagaactttctaaagtatttccggaagacatggctaagtatcgaagcatccggggggaggatcacccgccttcttaa

SEQ ID NO: 14 - protein, 105 aa, Homo sapiens
MGETEGKKDEADYKRLQTFPLVRHSDMPEEMRVETMELCVTACEKFSNNNESAAKMIKETMDKKFGSSWHVVIGEGFGFEITHEVKNLLYLYFGGTLAVCVWKCS

SEQ ID NO: 15 - DNA, 16 bp, Artificial Sequence
  anchored primer for PIG27
aagcttttttttttta

SEQ ID NO: 16 - DNA, 13 bp, Artificial Sequence
  H-AP12 primer for PIG27
aagcttgagtgct

SEQ ID NO: 17 - DNA, 1024 bp, Homo sapiens
gggtggctgaactctgatcttgacctagagtcatggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaaaataaaaatcccagaaggacaggagg

SEQ ID NO: 18 - protein, 321 aa, Homo sapiens
MAMATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 19 - DNA, 16 bp, Artificial Sequence
  anchored primer for PIG28
aagctttttttttttc

SEQ ID NO: 20 - DNA, 13 bp, Artificial Sequence
  H-AP12 primer for PIG28
aagcttgagtgct

SEQ ID NO: 21 - DNA, 2152 bp, Homo sapiens
ccaggatgtcggaggagatcatcacgccggtgtactgcactggggtgtcagcccaagtgcagaagcagcgggccagggagctgggcctgggccgccatgagaatgccatcaagtacctgggccaggattatgagcagctgcgggtgcgatgcctgcagagtgggaccctcttccgtgatgaggccttccccccggtaccccagagcctgggttacaaggacctgggtcccaattcctccaagacctatggcatcaagtggaagcgtcccacggaactgctgtcaaacccccagttcattgtggatggagctacccgcacagacatctgccagggagcactgggggactgctggctcttggcggccattgcctccctcactctcaacgacaccctcctgcaccgagtggttccgcacggccagagcttccagaatggctatgccggcatcttccatttccagctgtggcaatttggggagtgggtggacgtggtcgtggatgacctgctgcccatcaaggacgggaagctagtgttcgtgcactctgccgaaggcaacgagttctggagcgccctgcttgagaaggcctatgccaaggtaaatggcagctacgaggccctgtcagggggcagcacctcagagggctttgaggacttcacaggcggggttaccgagtggtacgagttgcgcaaggctcccagtgacctctaccagatcatcctcaaggcgctggagcggggctccctgctgggctgctccatagacatctccagcgttctagacatggaggccatcactttcaagaagttggtgaagggccatgcctactctgtgaccggggccaagcaggtgaactaccgaggccaggtggtgagcctgatccggatgcggaacccctggggcgaggtggagtggacgggagcctggagcgacagctcctcagagtggaacaacgtggacccatatgaacgggaccagctccgggtcaagatggaggacggggagttctggatgtcattccgagacttcatgcgggagttcacccgcctggagatctgcaacctcacacccgacgccctcaagagccggaccatccgcaaatggaacaccacactctacgaaggcacctggcggcgggggagcaccgcggggggctgccgaaactacccagccaccttctgggtgaaccctcagttcaagatccggctggatgagacggatgacccggacgactacggggaccgcgagtcaggctgcagcttcgtgctcgcccttatgcagaagcaccgtcgccgcgagcgccgcttcggccgcgacatggagactattggcttcgcggtctacgaggtccctccggagctggtgggccagccggccgtacacttgaagcgtgacttcttcctggccaatgcgtctcgggcgcgctcagagcagttcatcaacctgcgagaggtcagcacccgcttccgcctgccacccggggagtatgtggtggtgccctccaccttcgagcccaacaaggagggcgacttcgtgctgcgcttcttctcagagaagagtgctgggactgtggagctggatgaccagatccaggccaatctccccgatgagcaagtgctctcagaagaggagattgacgagaacttcaaggccctcttcaggcagctggcaggggaggacatggagatcagcgtgaaggagttgcggacaatcctcaataggatcatcagcaaacacaaagacctgcggaccaagggcttcagcctagagtcgtgccgcagcatggtgaacctcatggatcgtgatggcaatgggaagctgggcctggtggagttcaacatcctgtggaaccgcatccggaattacctgtccatcttccggaagtttgacctggacaagtcgggcagcatgagtgcctacgagatgcggatggccattgagtcggcaggcttcaagctcaacaagaagctgtacgagctcatcatcacccgctactcggagcccgacctggcggtcgactttgacaatttcgtttgctgcctggtgcggctagagaccatgttccgatttttcaaaactctggacacagatctggatggagttgtgacctttgacttgtttaagtggttgcagctgaccatgtttgcatgagg

SEQ ID NO: 22 - protein, 714 aa, Homo sapiens
MSEEIITPVYCTGVSAQVQKQRARELGLGRHENAIKYLGQDYEQLRVRCLQSGTLFRDEAFPPVPQSLGYKDLGPNSSKTYGIKWKRPTELLSNPQFIVDGATRTDICQGALGDCWLLAAIASLTLNDTLLHRVVPHGQSFQNGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 23 - DNA, 16 bp, Artificial Sequence
  anchored primer for PIG30
aagctttttttttttc

SEQ ID NO: 24 - DNA, 13 bp, Artificial Sequence
  H-AP33 primer for PIG30
aagcttgctgctc

SEQ ID NO: 25 - DNA, 2246 bp, Homo sapiens
cctcaggtttgccaataggattatcctgctgccatcatgtcttggtttgttgatcttgctggaaaggcagaagatcttttaaaccgagttgatcaaggggctgcaacagctctcagtaggaaagacaatgccagcaacatatatagcaaaaatactgactatactgaacttcaccagcaaaatacagatttgatatatcagactggacctaaatctacgtatatttcatcagcagctgataacattcgaaatcaaaaagccaccatcttagctggcactgcaaatgtgaaagtaggatctcggacaccagtagaggcctctcatcctgttgaaaatgcatctgttcctaggccttcatcccattttgtgcgaagaaaaaagtcagaacctgatgatgagctgctgtttgattttcttaatagttcacagaaggagcctaccgggagggtggaaatcagaaaggaaaaaggcaagacacctgtctttcagagctctcagacatcaagtgtcagttctgtgaaccccagtgtaaccaccatcaaaaccattgaagaaaattcttttgggagccaaacccacgaagctgccagtaactcagattctagccatgaaggtcaagaggaatcttcaaaggaaaatgtgtcatcaaatgctgcctgccctgaccacaccccaacacctaatgatgatggcaaatcacatgaactgtctaaccttcgactggagaatcagctgctgaggaatgaagttcagtctttaaatcaagaaatggcctcgttactccaaagatccaaagagactcaagaagaattaaacaaagcaagagcaagagttgaaaagtggaatgctgaccattcaaagagtgatcgaatgactcgaggactccgagcccaagtagatgacctgactgaagctgtggctgcaaaggattcccagctggctgtactgaaagtgagactccaggaagctgaccagctactgagtactcgcacagaagcattagaagccttacagagtgaaaaatcacgaataatgcaggatcaaagtgaaggtaacagcctgcagaatcaagctctgcagactcttcaggagagactgcatgaagcggatgccactctgaagagagagcaggagagctataaacagatgcagagcgagtttgctgcacgccttaataaagtggaaatggaacgtcagaatttagcagaagcaattacactggccgaaagaaaatactcagatgagaagaagagggttgatgaactgcagcagcaagtcaagctgtataagttgaacttggagtcctctaagcaggaattaattgactacaagcaaaaagctactagaatactgcaatctaaggaaaaattgattaacagcttgaaagaaggctctggttttgaaggcctagatagcagcactgccagtagcatggagctggaagaacttcggcatgagaaagagatgcagagggaggaaatacagaagctgatgggccagatacatcagctcagatccgaattacaggatatggaggcacagcaagttaatgaagcagaatcagcaagagaacagttacaggatctgcatgaccaaatagctgggcagaaagcatccaaacaagaactagagacagaactggagcgactgaagcaggagttccactatatagaagaagatctttatcgaacaaagaacacattgcaaagcagaattaaagatcgagacgaagaaattcaaaaactcaggaatcagcttaccaataaaactttaagcaatagcagtcagtctgagttagaaaatcgactccatcagctaacagagactctcatccagaaacagaccatgctggagagtctcagcacagaaaagaactccctggtctttcaactggagcgcctcgaacagcagatgaactccgcctctggaagtagtagtaatgggtcttcgattaatatgtctggaattgacaatggtgaaggcactcgtctgcgaaatgttcctgttctttttaatgacacagaaactaatctggcaggaatgtacggaaaagttcgcaaagctgctagttcaattgatcagtttagtattcgcctgggaatttttctccgaagataccccatagcgcgagtttttgtaattatatatatggctttgcttcacctctgggtcatgattgttctgttgacttacacaccagaaatgcaccacgaccaaccatatggcaaatgaaccaagcccagttg

SEQ ID NO: 26 - protein, 731 aa, Homo sapiens
MSWFVDLAGKAEDLLNRVDQGAATALSRKDNASNIYSKNTDYTELHQQNTDLIYQTGPKSTYISSAADNIRNQKATILAGTANVKVGSRTPVEASHPVENASVPRPSSHFVRRKKSEPDDELLFDFLNSSQKEPTGRVEIRKEKGKTPVFQSSQTSSVSSVNPSVTTIKTIEENSFGSQTHEAASNSDSSHEGQEESSKENVSSNAACPDHTPTPNDDGKSHELSNLRLENQLLRNEVQSLNQEMASLLQRSKETQEELNKARARVEKWNADHSKSDRMTRGLRAQVDDLTEAVAAKDSQLAVLKVRLQEADQLLSTRTEALEALQSEKSRIMQDQSEGNSLQNQALQTLQERLHEADATLKREQESYKQMQSEFAARLNKVEMERQNLAEAITLAERKYSDEKKRVDELQQQVKLYKLNLESSKQELIDYKQKATRILQSKEKLINSLKEGSGFEGLDSSTASSMELEELRHEKEMQREEIQKLMGQIHQLRSELQDMEAQQVNEAESAREQLQDLHDQIAGQKASKQELETELERLKQEFHYIEEDLYRTKNTLQSRIKDRDEEIQKLRNQLTNKTLSNSSQSELENRLHQLTETLIQKQTMLESLSTEKNSLVFQLERLEQQMNSASGSSSNGSSINMSGIDNGEGTRLRNVPVLFNDTETNLAGMYGKVRKAASSIDQFSIRLGIFLRRYPIARVFVIIYMALLHLWVMIVLLTYTPEMHHDQPYGK

SEQ ID NO: 27 - DNA, 16 bp, Artificial Sequence
  anchored primer for PIG31
aagctttttttttttg

SEQ ID NO: 28 - DNA, 13 bp, Artificial Sequence
  H-AP34 primer for PIG31
aagcttcagcagc

SEQ ID NO: 29 - DNA, 1973 bp, Homo sapiens
gcccctctggacactcataattcaatggtagatgcaggtggagttgagaacatcacccagcttccccaggagcttcctcagatgatggctgcagcagccgatggtttggggagtatagcgatagacacgacccagctcaacatgtccgtgacagatcccacagcctgggctacagccatgaataacctgggcatggttcccgtagggtcgcctggacagcagctcgtgtctgactcaatctgtgtcccaggctttgatccaagcctcaacatgatgactggaatcacccccattaacccaatgataccaggccttggactggtacctcccccaccaccaacagaagtggctgttgtcaaagaaataatccactgcaaaagctgtactctttttcctcaaaatccaaatcttccacctccttccacaagagaacgacctcctgggtgtaagaccgtgtttgtcggaggattaccagaaaatgctactgaggaaattattcaagaagtctttgaacagtgcggtgatattacagcaattcggaaaagcaagaagaatttttgtcacattcgctttgcagaggaattcatggttgataaagccatttacctttctggttataggatgcgattagggtctagcaccgacaaaaaggattcaggccgccttcatgtggactttgcccaggccagggatgacttctatgagtgggaatgcaagcagaggatgcgtgcccgggaggagcggcaccggcgcaagctggaggaggaccggctcaggcccccatccccgcctgccataatgcactactcggagcacgaagccgctctgctggctgaaaagctgaaagatgatagcaagtcttcagaggctatcacagtgctgctttcctggattgaacgaggggaagtgaatcggcgctctgcaaaccagttctattccatggtgcagtcggccaacagccacgtccgccggctaatgaatgaaaaagccacccatgagcaagagatggaggaagccaaggagaattttaaaaatgccttaactgggattctcactcaatttgagcagattgtggccgttttcaacgcttctaccagacaaaaagcttgggaccatttctcgaaagcccagcgcaagaacatagacatttggcgaaagcattctgaggagctccggaatgctcaaagtgagcagctcatgggcatccgccgcgaagaagaaatggaaatgtctgatgatgagaactgtgacagccctacaaagaaaatgagagtcgatgaatcagccctggctgcccaggcctacgctctgaaagaggagaatgacagtctccgctggcagctggatgcctacaggaatgaggtggagctgctgaaacaagaaaaagaacagcttttccgaacagaagaaaacctcaccaaggaccagcaactgcagtttctgcagcaaaccatgcaaggcatgcagcagcaattgctaaccatccaggaggagttaaacaacaaaaagtcagaattggaacaagcaaaggaagagcagtcccatacacaagcgttactaaaagtcctgcaggaacaattaaaaggtaccaaggaattggtcgagaccaatggccacagccatgaggattcaaatgaaatcaatgtgttgacagttgcattagtcaaccaagaccgagagaacaatattgagaaaagaagccaaggcttaaaatcagagaaagaagctctactaataggtatcatatcaacgtttcttcacgtccatccttttggagccaacatagaatatctttggtcatacatgcagcagctggactccaagatatctgcaaatgaaatagaaatgcttttgatgaggctgccacgcatgttcaaacaggaattcacgggtgtgggagccacgctggaaaaaagatggaagctgtgtgcctttgaaggaattaaaactacctaactgcgaagagcaaagca

SEQ ID NO: 30 - protein, 643 aa, Homo sapiens [sequence omitted]

SEQ ID NO: 31 - DNA, 16 bp, Artificial Sequence
  anchored primer for PIG38
aagctttttttttttc

SEQ ID NO: 32 - DNA, 13 bp, Artificial Sequence
  H-AP10 primer for PIG38
aagcttccacgta

SEQ ID NO: 33 - DNA, 1586 bp, Homo sapiens
ggagggtggtgtccactgcccagttccgtgtcccgatgcccagcgccagcgccagccgcaagagtcaggagaagccgcgggagatcatggacgcggcggaagattatgctaaagagagatatggaatatcttcaatgatacaatcacaagaaaaaccagatcgagttttggttcgggttagagacttgacaatacaaaaagctgatgaagttgtttgggtacgtgcaagagttcatacaagcagagctaaagggaaacagtgcttcttagtcctacgtcagcagcagtttaatgtccaggctcttgtggcggtgggagaccatgcaagcaagcagatggttaaatttgctgccaacatcaacaaagagagcattgtggatgtagaaggtgttgtgagaaaagtgaatcagaaaattggaagctgtacacagcaagacgttgagttacatgttcagaagatttatgtgatcagtttggctgaaccccgtctgcccctgcagctggatgatgctgttcggcctgaggcagaaggagaagaggaaggaagagctactgttaaccaggatacaagattagacaacagagtcattgatcttaggacatcaactagtcaggcagtcttccgtctccagtctggcatctgccatctcttccgagaaactttaattaacaaaggttttgtggaaatccaaactcctaaaattatttcagctgccagtgaaggaggagccaatgtttttactgtgtcatattttaaaaataatgcatacctggctcagtccccacagctatataagcaaatgtgcatttgtgctgattttgagaaggttttctctattggaccagtattcagagcggaagactctaatacccatagacatctaactgagtttgttggtttggacattgaaatggcttttaattaccattaccacgaagttatggaagaaattgctgacaccatggtacacatattcaaaggacttcaagaaaggtttcagactgaaattcaaacagtgaataaacagttcccatgtgagccattcaaatttttggagccaactctaagactagaatattgtgaagcattggctatgcttagggaagctggagtcgaaatgggagatgaagacgatctgagcacaccaaatgaaaagctgttgggtcatttggtaaaggaaaagtatgatacagatttttatattcttgataaatatccattggctgtaagacctttctataccatgcctgacccaagaaatcccaaacagtccaactcttacgatatgttcatgagaggagaagaaatattgtcaggagctcaaagaatacatgatcctcaactgctaacagagagagctttacatcatggaattgatttggagaaaattaaggcttacattgattccttccgctttggagcccctcctcatgctggtggaggcattggattggaacgagttactatgctgtttctgggattgcataatgttcgtcagacctccatgttccctcgtgatcccaaacgactcactccttaaattcacactttgccacttaactccagtgtggatgacagagcgaga

SEQ ID NO: 34 - protein, 501 aa, Homo sapiens [sequence omitted]

SEQ ID NO: 35 - DNA, 16 bp, Artificial Sequence
  anchored primer for PIG40
aagctttttttttttc

SEQ ID NO: 36 - DNA, 13 bp, Artificial Sequence
  H-AP11 primer for PIG40
aagcttcgggtaa

SEQ ID NO: 37 - DNA, 1245 bp, Homo sapiens
aagagggcaaccctaacgatacgcttgactttctgtggctgggaacaccttccaccatgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctccagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatagacatggtgagcaaccagcacccctggtttggcatggagcaggagtataccctcatggggacagatgggcacccctttggttggccttccaacggcttccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaattaagtggactagacctccagctgttgagcccctcctagttcttcatcccactccaactcttccccctctca

SEQ ID NO: 38 - protein, 233 aa, Homo sapiens [sequence omitted]

SEQ ID NO: 39 - DNA, 16 bp, Artificial Sequence
  anchored primer for PIG43
aagctttttttttttg

SEQ ID NO: 40 - DNA, 13 bp, Artificial Sequence
  H-AP11 primer for PIG43
aagcttcgggtaa

SEQ ID NO: 41 - DNA, 1721 bp, Homo sapiens
ggcaaggatcttgttccaaatcatctttcatattacctttataatcatgtggctatgtggccggaacaaagtgacaaatggcctacagctgtgagagcaaatggacatctcctcctgaactctgagaagatgtcaaaatccacaggcaacttcctcactttgacccaagctattgacaaattttcagcagatggaatgcgtttggctctggctgatgctggtgacactgtagaagatgccaactttgtggaagccatggcagatgcaggtattctccgtctgtacacctgggtagagtgggtgaaagaaatggttgccaactgggacagcctaagaagtggtcctgccagcactttcaatgatagagtttttgccagtgaattgaatgcaggaattataaaaacagatcaaaactatgaaaagatgatgtttaaagaagctttgaaaacagggttttttgagtttcaggccgcaaaagataagtaccgtgaattggctgtggaagggatgcacagagaacttgtgttccggtttattgaagttcagacacttctcctcgctccattctgtccacatttgtgtgagcacatctggacactcctgggaaagcctgactcaattatgaatgcttcatggcctgtggcaggtcctgttaatgaagttttaatacactcctcacagtatcttatggaagtaacacatgaccttagactacgactcaagaactatatgatgccagctaaagggaagaagactgacaaacaacccctgcagaagccctcacattgcaccatctatgtggcaaagaactatccaccttggcaacataccaccctgtctgttctacgtaaacactttgaggccaataacggaaaactgcctgacaacaaagtcattgctagtgaactaggcagtatgccagaactgaagaaatacatgaagaaagtcatgccatttgttgccatgattaaggaaaatctggagaagatggggcctcgtattctggatttgcaattagaatttgatgaaaaggctgtgcttatggagaatatagtctatctgactaattcgcttgagctagaacacatagaagtcaagtttgcctccgaagcagaagataaaatcagggaagactgctgtcctgggaaaccacttaatgtttttagaatagaacctggtgtgtccgtttctctggtgaatccccagccatccaatggccacttctcaaccaaaattgaaatcaggcaaggagataactgtgattccataatcaggcgtttaatgaaaatgaatcgaggaattaaagacctttccaaagtgaaactgatgagatttgatgatccactgttggggcctcgacgagttcctgtcctgggaaaggagtacaccgagaagacccccatttctgagcatgctgttttcaatgtggacctcatgagcaagaaaattcatctgactgagaatgggataagggtggatattggcgatacaataatctatctggttcattaaactcatgcacattggagatttatcctggtttcttaggaatactactactctgattgtgtctactgattggctatcagaaccttaggctggacctaaatagattgatttcatttctaaccatccaattctgcatgtattcataattctatcaagtcatctttgattcctggacctaataaattttttttccctttctttgggtgtccaag

SEQ ID NO: 42 - protein, 485 aa, Homo sapiens
MWPEQSDKWPTAVRANGHLLLNSEKMSKSTGNFLTLTQAIDKFSADGMRLALADAGDTVEDANFVEAMADAGILRLYTWVEWVKEMVANWDSLRSGPASTFNDRVFASELNAGIIKTDQNYEKMMFKEALKTGFFEFQAAKDKYRELAVEGMHRELVFRFIEVQTLLLAPFCPHLCEHIWTLLGKPDSIMNASWPVAGPVNEVLIHSSQYLMEVTHDLRLRLKNYMMPAKGKKTDKQPLQKPSHCTIYVAKNYPPWQHTTLSVLRKHFEANNGKLPDNKVIASELGSMPELKKYMKKVMPFVAMIKENLEKMGPRILDLQLEFDEKAVLMENIVYLTNSLELEHIEVKFASEAEDKIREDCCPGKPLNVFRIEPGVSVSLVNPQPSNGHFSTKIEIRQGDNCDSIIRRLMKMNRGIKDLSKVKLMRFDDPLLGPRRVPVLGKEYTEKTPISEHAVFNVDLMSKKIHLTENGIRVDIGDTIIYLVH

SEQ ID NO: 43 - DNA, 16 bp, Artificial Sequence
  anchored primer for PIG44
aagctttttttttttc

SEQ ID NO: 44 - DNA, 13 bp, Artificial Sequence
  H-AP23 primer for PIG44
aagcttggctatg

SEQ ID NO: 45 - DNA, 1312 bp, Homo sapiens
cagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaagcagg

SEQ ID NO: 46 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 47 - DNA, 16 bp, Artificial Sequence
  anchored primer for PIG46
aagcttttttttttta

SEQ ID NO: 48 - DNA, 13 bp, Artificial Sequence
  H-AP24 primer for PIG46
aagcttcactagc

SEQ ID NO: 49 - DNA, 827 bp, Homo sapiens
ttctcctatcggggtcacagggacgctaagattgctacctggactttcgttgaccatgctgtcccgggtggtactttccgccgccgccacagcggccccctctctgaagaatgcagccttcctaggtccaggggtattgcaggcaacaaggacctttcatacagggcagccacaccttgtccctgtaccacctcttcctgaatacggaggaaaagttcgttatggactgatccctgaggaattcttccagtttctttatcctaaaactggtgtaacaggaccctatgtactcggaactgggcttatcttgtacgctttatccaaagaaatatatgtgattagcgcagagaccttcactgccctatcagtactaggtgtaatggtctatggaattaaaaaatatggtccctttgttgcagactttgctgataaactcaatgagcaaaaacttgcccaactagaagaggcgaagcaggcttccatccaacacatccagaatgcaattgatacggagaagtcacaacaggcactggttcagaagcgccattacctttttgatgtgcaaaggaataacattgctatggctttggaagttacttaccgggaacgactgtatagagtatataaggaagtaaagaatcgcctggactatcatatatctgtgcagaacatgatgcgtcgaaaggaacaagaacacatgataaattgggtggagaagcacgtggtgcaaagcatctccacacagcaggaaaaggagacaattgccaagtgcattgcggacctaaagctgctggcaaagaaggctcaagcacagccagttatgtaaa

SEQ ID NO: 50 - protein, 256 aa, Homo sapiens
MLSRVVLSAAATAAPSLKNAAFLGPGVLQATRTFHTGQPHLVPVPPLPEYGGKVRYGLIPEEFFQFLYPKTGVTGPYVLGTGLILYALSKEIYVISAETFTALSVLGVMVYGIKKYGPFVADFADKLNEQKLAQLEEAKQASIQHIQNAIDTEKSQQALVQKRHYLFDVQRNNIAMALEVTYRERLYRVYKEVKNRLDYHISVQNMMRRKEQEHMINWVEKHVVQSISTQQEKETIAKCIADLKLLAKKAQAQPVM

SEQ ID NO: 51 - DNA, 16 bp, Artificial Sequence
  anchored primer for PIG47
aagctttttttttttc

SEQ ID NO: 52 - DNA, 13 bp, Artificial Sequence
  H-AP5 primer for PIG47
aagcttagtaggc

SEQ ID NO: 53 - DNA, 1707 bp, Homo sapiens
cagaaggttctgccggttcccccagctctgggtacccggctctgcatcgcgtcgccatgatgggccatcgtccagtgctcgtgctcagccagaacacaaagcgtgaatccggaagaaaagttcaatctggaaacatcaatgctgccaagactattgcagatatcatccgaacatgtttgggacccaagtccatgatgaagatgcttttggacccaatgggaggcaatgtgatgaccaatgatggcaatgccattcttcgagagattcaagtccagcatccagcggccaagtccatgatcgaaattagccggacccaggatgaagaggttggagatgggaccacatcagtaattattcttgcaggggaaatgctgtctgtagctgagcacttcctggagcagcagatgcacccaacagtggtgatcagtgcttaccgcaaggcattggatgatatgatcagcaccctaaagaaaataagtatcccagtcgacatcagtgacagtgatatgatgctgaacatcatcaacagctctattactaccaaagccatcagtcggtggtcatctttggcttgcaacattgccctggatgctgtcaagatggtacagtttgaggagaatggtcggaaagagattgacataaaaaaatatgcaagagtggaaaagatacctggaggcatcattgaagactcctgtgtcttgcgtggagtcatgattaacaaggacgtgacccatccacgtatgtggcgctatatcaagaaccctcgcattgtgctgctggattcttctctggagtacaagaaaggagaaagccagactgacattgagattacacgagaggaggacttcacccgaattctccagatggaggaagagtacatccagcagctctgtgaggacattatccaactgaagcccgatgtggtcatcactgaaaagggcatctcagatttagctcagcactaccttatgcgggccaatatcacagccatccgcagagtccggaagacagacaataatcgcattgctagagcctgtggggcccggatagtcagccgaccagaggaactgagagaagatgatattggaacaggagcaggcctgttggaaatcaagaaaattggagatgaatactttactttcatcactgactgcaaagaccccaaggcctgcaccattctcctccggggggctagcaaagagattctctcggaagtagaacgcaacctccaggatgccatgcaagtgtgtcgcaatgttctcctggaccctcagctggtgccagggggtggggcctccgagatggctgtggcccatgccttgacagaaaaatccaaggccatgactggtgtggaacaatggccatacagggctgttgcccaggccctagaggtcattcctcgtaccctgatccagaactgtggggccagcaccatccgtctacttacctcccttcgggccaagcacacccaggagaactgtgagacctggggtgtaaatggtgagacgggtactttggtggacatgaaggaactgggcatatgggagccattggctgtgaagctgcagacttataagacagcagtggagacggcagttctgctactgcgaattgatgacatcgtttcaggccacaaaaagaaaggcgatgaccagagccggcaaggcggggctcctgatgctggccaggagtgagtgctaggcaagg

SEQ ID NO: 54 - protein, 545 aa, Homo sapiens [sequence omitted]

SEQ ID NO: 55 - DNA, 16 bp, Artificial Sequence
  anchored primer for PIG48
aagcttttttttttta

SEQ ID NO: 56 - DNA, 13 bp, Artificial Sequence
  H-AP7 primer for PIG48
aagcttaacgagg

SEQ ID NO: 57 - DNA, 643 bp, Homo sapiens
catgcagagagcttcacgtctgaagagagagctgcacatgttagccacagagccacccccaggcatcacatgttggcaagataaagaccaaatggatgacctgcgagctcaaatattaggtggagccaacacaccttatgagaaaggtgtttttaagctagaagttatcattcctgagaggtacccatttgaacctcctcagatccgatttctcactccaatttatcatccaaacattgattctgctggaaggatttgtctggatgttctcaaattgccaccaaaaggtgcttggagaccatccctcaacatcgcaactgtgttgacctctattcagctgctcatgtcagaacccaaccctgatgacccgctcatggctgacatatcctcagaatttaaatataataagccagccttcctcaagaatgccagacagtggacagagaagcatgcaagacagaaacaaaaggctgatgaggaagagatgcttgataatctaccagaggctggtgactccagagtacacaactcaacacagaaaaggaaggccagtcagctagtaggcatagaaaagaaatttcatcctgatgtttaggggacttgtcctggttcatcttagttaatgtgttctttgccaaggtga

SEQ ID NO: 58 - protein, 197 aa, Homo sapiens
MQRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLPEAGDSRVHNSTQKRKASQLVGIEKKFHPDV

SEQ ID NO: 59 - DNA, 16 bp, Artificial Sequence
  anchored primer for PIG50
aagctttttttttttc

SEQ ID NO: 60 - DNA, 13 bp, Artificial Sequence
  H-AP5 primer for PIG50
aagcttagtaggc

SEQ ID NO: 61 - DNA, 1936 bp, Homo sapiens
ccggtagaggactgtgaaccaaaagttgtcccccaggatggacttcaccgcgcagcccaagcctgccactgccctctgtggcgtcgtgagtgccgacgggaagatcgcttaccctccgggggtaaaagagatcaccgacaagatcaccacggacgagatgatcaaacgcctgaagatggtagtgaaaacctttatggatatggatcaggactcagaagatgaaaaacagcagtatctcccactagccttgcatcttgcatctgaattcttcctcaggaaccccaataaagatgtgcgtctccttgtagcatgttgtttggctgatatctttcgtatctatgccccagaagctccatatacttcccatgataaacttaaggacatatttttgtttattaccagacaattaaaaggtttggaggatacaaagagtccacagtttaatagatacttttatttattagagaatttagcttgggttaaatcatataacatctgctttgaattggaagattgcaatgaaatttttattcagctttttagaactctcttctcagtgatcaacaatagccacaataagaaggtacaaatgcacatgctagatttgatgagttctatcatcatggaaggtgatggagttactcaagaattattggactccattcttattaacctcattcctgcacataagaacttaaataaacagtcctttgaccttgcaaaagtgctattgaaaagaacagtccagactattgaggcatgcattgctaattttttcaatcaagtcctggtgctgggaagatcatcagtaagtgatttgtcagaacatgtatttgatctgattcaggaactttttgctatagatcctcatttattattatccgtcatgccacagcttgaattcaaactaaagagcaatgatggagaagagcgattagctgttgttcgacttctagctaaattgtttggctccaaagattctgatttggcaacacagaatcgtcctctttggcaatgttttcttggacgatttaatgatattcatgttcctgtgagattagaaagtgtgaaatttgccagtcattgtttaatgaatcacccagatttagcgaaggatctcacagaatatttaaaggttagatcacatgatccagaagaagctattcgtcatgatgtcattgttactataataacagctgccaagagggacctggccttagtaaatgatcagctgcttggctttgtaagggaaagaacactggataaacggtggcgagtaagaaaagaagctatgatgggtctggctcagctttataagaaatactgtcttcatggtgaagcaggaaaggaagctgcagagaaagtcagctggataaaggacaaacttctgcatatttattatcagaacagcattgacgacaaactgttggtagagaaaatctttgctcggtatcttgtcccccacaacctggaaacagaagagagaatgaaatgcttatattacttatatgctagtttggatccaaatgctgtaaaagctctcaacgaaatgtggaagtgtcagaacatgcttcggagccatgtacgcgaactattggatttgcacaagcagcctacatcagaggctaactgttctgccatgtttggaaaactgatgaccatagcaaagaatttgcctgaccccgggaaggcacaagattttgtgaagaaatttaaccaggttctcggcgatgatgagaaacttcggtctcagttggagttattaattagcccaacctgttcttgcaaacaagcagatatttgtgtggttagtaaatcatacttcacactttttctctagctttacttacagaaatatgtattttattaaagcctgttttacatacagtgatttgtgtaaatgtaacaatattgtgagctctgaattactttggag

SEQ ID NO: 62 - protein, 600 aa, Homo sapiens [sequence omitted]

SEQ ID NO: 63 - DNA, 16 bp, Artificial Sequence
  anchored primer for PIG54
aagcttttttttttta

SEQ ID NO: 64 - DNA, 13 bp, Artificial Sequence
  H-AP15 primer for PIG54
aagcttacgcaac

SEQ ID NO: 65 - DNA, 526 bp, Homo sapiens
cgcttctctgcactatgtcgggtggcctcctgaaggcgctgcgcagcgactcctacgtggagctgagccagtaccgggaccagcacttccggggtgacaatgaagaacaagaaaaattactgaagaaaagctgtacgttatatgttggaaatctttctttttacacaactgaagaacaaatctatgaactcttcagcaaaagtggtgacataaagaaaatcattatgggtctggataaaatgaagaaaacagcatgtggattctgttttgtggaatattactcacgcgcagatgcggaaaacgccatgcggtacataaatgggacgcgtctggatgaccgaatcattcgcacagactgggacgcaggctttaaggagggcaggcaatacggccgtgggcgatctgggggccaggttcgggatgagtatcggcaggactacgatgctgggagaggaggctatggaaaactggcacagaaccagtgagtggtgagagctctgtcagtgacaaacactcctttggcctg

SEQ ID NO: 66 - protein, 156 aa, Homo sapiens
MSGGLLKALRSDSYVELSQYRDQHFRGDNEEQEKLLKKSCTLYVGNLSFYTTEEQIYELFSKSGDIKKIIMGLDKMKKTACGFCFVEYYSRADAENAMRYINGTRLDDRIIRTDWDAGFKEGRQYGRGRSGGQVRDEYRQDYDAGRGGYGKLAQNQ

SEQ ID NO: 67 - DNA, 16 bp, Artificial Sequence
  anchored primer for PIG55
aagctttttttttttg

SEQ ID NO: 68 - DNA, 13 bp, Artificial Sequence
  H-AP4 primer for PIG55
aagcttctcaacg

SEQ ID NO: 69 - DNA, 1008 bp, Homo sapiens
atggcggtggacatcacgctgctattccgggccagcgtcaagaccgtgaagacgcggaacaaggcgctgggagtggcggtgggcggcggggtcgatggcagccgggacgagctgttccgccggagcccccggcccaagggcgacttctccagccgggcccgcgaagtgatttctcacattggcaaactgagagattttcttctggaacacaggaaagattatattaatgcttatagccataccatgtctgaatatgggaggatgacagacacagaacgagaccagatagaccaggatgcccagatattcatgaggacctgttcagaagcaattcagcaactacgaacagaagctcacaaggagatacattcccagcaagtgaaggagcacaggaccgctgttttggatttcattgaagattacttgaaaagagtatgtaaactttactcagaacagagagccatccgagttaaaagagtggtggataagaaaagattatctaagctggaaccagaaccaaatacaaagacaagagaatccacatcttctgagaaagtttcacagagtccttcaaaagactctgaagaaaaccctgccactgaagaacgtccagaaaaaattttggctgaaacacaacctgaattgggaacgtggggagatggcaaaggcgaagatgagttatccccagaagaaatacaaatgtttgaacaggaaaatcagcgactaattggtgaaatgaacagcttgtttgatgaagtgaggcaaatcgaagggagagtggttgagatttccagactccaagagatattcacggaaaaggttttgcaacaggaagctgagattgacagcattcaccagttagttgtgggggcaactgaaaatatcaaggaaggcaacgaagacataagagaggccattaaagacaacgctggcttccgcgtgtggatcctcttcttcctcgtgatgtgctccttctccttgctcttcctcgactggtacgacagctag

SEQ ID NO: 70 - protein, 335 aa, Homo sapiens [sequence omitted]

SEQ ID NO: 71 - DNA, 16 bp, Artificial Sequence
  anchored primer for GIG9
aagcttttttttttta

SEQ ID NO: 72 - DNA, 13 bp, Artificial Sequence
  H-AP14 primer for GIG9
aagcttggagctt

SEQ ID NO: 73 - DNA, 1382 bp, Homo sapiens
agcagggtcaccatttgcagcgcaacatggcaggagctggaggagggaatgatattcagtggtgtttttctcaggtgaaaggagcagtagatgatgatgtagcagaagcagatataatttctacagtagaatttaatcattctggagaattactagcaacaggagataaaggtggtagagttgtcatctttcaacaggagcaggagaacaaaatccagtctcatagcagaggagaatataatgtttacagcaccttccagagccatgaaccagagtttgactacttgaaaagtttagaaatagaagaaaagatcaacaaaattaggtggttaccccagaaaaatgctgctcagtttttattgtctaccaatgataaaacaataaaattatggaaaatcagtgaaagggacaaaagaccagaagggtataacttgaaagaggaggatggaaggtatagagatcctactacagttactacactacgagtgccagtctgtaggcctatggatctaatggttgaggccagtccacgaagaatatttgccaatgctcatacatatcacatcaactcaatttctattaatagtgattatgaaacatatttatctgcagatgatttgcggattaatctttggcatctggaaattacagacaggagttttaacattgtggatatcaagcctgccaatatggaagagctaacagaggtgattacagcagcagaatttcatccaaacagctgtaacacatttgtatacagcagcagtaaaggaactattcggctatgtgacatgagggcatctgccctctgtgatagacattctaaattgtttgaagaacctgaagatcccagtaacaggtcatttttttccgaaatcatctcctctatttcggatgtaaaattcagccatagtggtcgatacatgatgactagagactatttgtcagtcaaaatttgggacttaaatatggaaaacaggcctgtggaaacataccaggtgcatgaatacctcagaagtaaactctgttcactgtacgaaaatgactgcatatttgacaaatttgaatgttgttggaatggatctgacagtgttgtcatgactggatcttacaataatttcttcagaatgtttgacagaaacacaaagcgagacataaccctagaagcatcgcgggaaaacaataagcctcgcacagttctgaagcctcgcaaagtctgtgcaagtggcaagcgaaagaaagatgaaataagtgttgacagcctagacttcaataagaaaatccttcacacagcctggcaccccaaggaaaatatcattgccgtagctactacaaacaatctgtatatatttcaagacaaagtgaattagggttggcattcc

SEQ ID NO: 74 - protein, 447 aa, Homo sapiens [sequence omitted]

SEQ ID NO: 75 - DNA, 16 bp, Artificial Sequence
  anchored primer for HLC-9
aagctttttttttttg

SEQ ID NO: 76 - DNA, 13 bp, Artificial Sequence
  H-AP7 primer for HLC-9
aagcttaacgagg

SEQ ID NO: 77 - DNA, 1301 bp, Homo sapiens
atatggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttgaggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccagtgaagaaacaccacccgtccagtaccaccaaagtagttctctgtcatgtgtgttccctgc

SEQ ID NO: 78 - protein, 413 aa, Homo sapiens [sequence omitted]

SEQ ID NO: 79 - DNA, 16 bp, Artificial Sequence
  anchored primer for GIG18
aagctttttttttttc

SEQ ID NO: 80 - DNA, 13 bp, Artificial Sequence
  H-AP4 primer for GIG18
aagcttctcaacg

SEQ ID NO: 81 - DNA, 749 bp, Homo sapiens
gcgactgaagcagcatggccaagccgtgtggggtgcgcctgagcggggaagcccgcaaacaggtggaggtcttcaggcagaatcttttccaggaggctgaggaattcctctacagattcttgccacagaaaatcatatacctgaatcagctcttgcaagaggactccctcaatgtggctgacttgacttccctccgggccccactggacatccccatcccagaccctccacccaaggatgatgagatggaaacagataagcaggagaagaaagaagtccctaagtgtggatttctccctgggaatgagaaagtcctgtccctgcttgccctggttaagccagaagtctggactctcaaagagaaatgcattctggtgattacatggatccaacacctgatccccaagattgaagatggaaatgattttggggtagcaatccaggagaaggtgctggagagggtgaatgccgtcaagaccaaagtggaagctttccagacaaccatttccaagtacttctcagaacgtggggatgctgtggccaaggcctccaaggagacccatgtaatggattaccgggccttggtgcatgagcgagatgaggcagcctatggggagctcagggccatggtgctggacctgagggccttctatgctgagctttatcatatcatcagcagcaacctggagaaaattgtcaccccaaagggtgaagaaaagccatctatgtactgaacccgggactagaag

SEQ ID NO: 82 - protein, 239 aa, Homo sapiens [sequence omitted]

SEQ ID NO: 83 - DNA, 16 bp, Artificial Sequence
  anchored primer for MIG22
aagcttttttttttta

SEQ ID NO: 84 - DNA, 13 bp, Artificial Sequence
  H-AP6 primer for MIG22
aagcttgcaccat

(19) United States
(12) Patent Application Publication
(10) Pub. No.: US 2009/0221794 A1
(43) Pub. Date: Sep. 3, 2009

(54) Human Protooncogene and Protein Encoded By Same

(76) Inventors: Hyun-Kee Kim, Seoul (KR); Jin-Woo Kim, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 11/910,010

(22) PCT Filed: Mar. 30, 2006
(86) PCT No.: PCT/KR06/01176

§ 371 Date: Sep. 28, 2007

(30) Foreign Application Priority Data
Mar. 30, 2005 (KR) 10-2005-0026244

(51) Int. Cl. C07K 14/00; C12N 15/11

(52) U.S. Cl. 530/350; 536/23.5

(57) ABSTRACT

Disclosed are a novel protooncogene and a protein encoded by the same. The protooncogene of the present invention may be effectively used for diagnosing various cancers including breast cancer, leukemia, uterine cancer, malignant lymphoma, etc.

### TECHNICAL FIELD

The present invention relates to a novel protooncogene exhibiting an ability to induce carcinogenesis and cancer metastasis, and a protein encoded by the same.

### BACKGROUND ART

Generally, it has been known that the higher animals, including human, have approximately 30,000 genes, but only approximately 15% of the genes are expressed in each subject. Accordingly, it was found that all phenomena of life, namely development, differentiation, homeostasis, responses to stimulus, control of cell cycle, aging and apoptosis (a programmed cell death), etc. were determined depending on what genes are selected and expressed (Liang, P. and A. B. Pardee, Science 257: 967-971, 1992).

The pathological phenomena such as oncogenesis are induced by the genetic variation, resulting in changed expression of genes. Accordingly, it is thought that the comparison of gene expressions between different cells is a basic and fundamental approach to understand various biological mechanisms.

For example, the mRNA differential display method proposed by Liang and Pardee (Liang, P. and A. B. Pardee, see the above reference) has been effectively used for searching tumor suppressor genes, genes relevant to cell cycle regulation, and transcriptional regulatory genes relevant to apoptosis, etc., and also widely employed for specifying correlations of the various genes that appear only in one cell.

Putting together the various results of oncogenesis, it has been reported that various genetic changes such as loss of specific chromosomal heterozygosity, activation of protooncogenes, and inactivation of other tumor suppressor genes including the p53 gene were accumulated in the tumor tissues, resulting in development of human tumors (Bishop, J. M., Cell 64: 235-248, 1991; Hunter, T., Cell 64: 249-270, 1991). Also, it was reported that 10 to 30% of the cancer was induced if protooncogenes are activated by amplifying the protooncogenes.

The activation of protooncogenes plays an important role in the etiological studies of many cancers, and therefore there have been attempts to specify the role.

Accordingly, the present inventors found that a mechanism for generating breast cancer was studied at a protooncogene level, and therefore the protooncogene, named a human proliferation-inducing gene (PIG), showed a specifically increased level of expression only in the cancer cell. The protooncogene may be effectively used for diagnosing, preventing and treating various cancers such as breast cancer, leukemia, uterine cancer, malignant lymphoma, etc.

### DISCLOSURE OF INVENTION

Accordingly, the present invention is designed to solve the problems of the prior art, and therefore it is an object of the present invention to provide a protooncogene or its fragments.

It is another object of the present invention to provide a recombinant vector containing the protooncogene or its fragments; and a microorganism transformed by the recombinant vector.

It is still another object of the present invention to provide a protein encoded by the protooncogene; or its fragments.

It is still another object of the present invention to provide a kit for diagnosing cancer, including the protooncogene or its fragments.

It is yet another object of the present invention to provide a kit for diagnosing cancer, including the protein or its fragments.

In order to accomplish one of the above objects, the present invention provides a protooncogene having a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29; SEQ ID NO: 33 SEQ ID NO: 37; SEQ ID NO: 41; SEQ ID NO: 45; SEQ ID NO: 49; SEQ ID NO: 53; SEQ ID NO: 57; SEQ ID NO: 61; SEQ ID NO: 65; SEQ ID NO: 69; SEQ ID NO: 73; SEQ ID NO: 77; and SEQ ID NO: 81; and fragments thereof.

According to another of the above objects, the present invention provides a recombinant vector containing the protooncogene or its fragments; and a microorganism transformed by the recombinant vector.

According to still another of the above objects, the present invention provides a protein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; SEQ ID NO: 58; SEQ ID NO: 62; SEQ ID NO: 66; SEQ ID NO: 70; SEQ ID NO: 74; SEQ ID NO: 78; and SEQ ID NO: 82; and fragments thereof, the protein and the fragments thereof being encoded by the protooncogenes, respectively.

According to still another of the above objects, the present invention provides a kit for diagnosing cancer including the protooncogene or its fragments.

According to yet another of the above objects, the present invention provides a kit for diagnosing cancer including the protooncoprotein or its fragments.

Hereinafter, preferable embodiments of the present invention will be described in detail referring to the accompanying drawings.

1. PIG12

The protooncogene, a human proliferation-inducing gene 12 (PIG12), of the present invention (hereinafter, referred to as a PIG12 protooncogene) has a 1,258-bp full-length DNA sequence set forth in SEQ ID NO: 1.

In the DNA sequence of SEQ ID NO: 1, an open reading frame corresponding to nucleotide sequence positions from 68 to 1,252 (1,250-1,252: a stop codon) is a full-length protein coding region, and an amino acid sequence derived from the protein coding region is set forth in SEQ ID NO: 2 and contains 394 amino acids (“a PIG12 protein”).

A protein expressed from the protooncogene of the present invention contains 394 amino acids and has an amino acid sequence set forth in SEQ ID NO: 2 and a molecular weight of approximately 46 kDa.

2. PIG18

The protooncogene, a human proliferation-inducing gene 18 (PIG18), of the present invention (hereinafter, referred to as a PIG18 protooncogene) has a 1,024-bp full-length DNA sequence set forth in SEQ ID NO: 5.

In the DNA sequence of SEQ ID NO: 5, an open reading frame corresponding to nucleotide sequence positions from 875 to 1,063 (1,061-1,063: a stop codon) is a full-length protein coding region, and an amino acid sequence derived from the protein coding region is set forth in SEQ ID NO: 6 and contains 62 amino acids (hereinafter, referred to as “a PIG18A protein”).

The DNA sequence of SEQ ID NO: 5 has been deposited with Accession No. AY771596 in the GenBank database of U.S. National Institutes of Health (NIH) (Scheduled Release Date: Dec. 31, 2005), and the DNA base sequence result revealed that its DNA sequence was similar to that of the Homo sapiens coagulation factor II (thrombin) receptor (F2R) gene deposited with Accession No. NM_{—}001992 in the database. From this study result, it was however found that the PIG18 protooncogene is highly expressed in various human tumors including the uterine cancer, while its expression is significantly reduced in various normal tissues.

A protein expressed from the protooncogene of the present invention contains 62 amino acids and has an amino acid sequence set forth in SEQ ID NO: 6 and a molecular weight of approximately 7 kDa.

3. PIG23

The protooncogene, a human proliferation-inducing gene 23 (PIG23), of the present invention (hereinafter, referred to as a PIG23 protooncogene) has a 2,150-bp full-length DNA sequence set forth in SEQ ID NO: 9.

In the DNA sequence of SEQ ID NO: 9, an open reading frame corresponding to nucleotide sequence positions from 25 to 1,953 (1,951-1,953: a stop codon) is a full-length protein coding region, and an amino acid sequence derived from the protein coding region is set forth in SEQ ID NO: 10 and contains 642 amino acids (hereinafter, referred to as “a PIG23 protein”).

The DNA sequence of SEQ ID NO: 9 has been deposited with Accession No. AY826819 in the GenBank database of U.S. National Institutes of Health (NIH) (Scheduled Release Date: Dec. 31, 2005), and the DNA base sequence result revealed that its DNA sequence was similar to that of the protein inhibitor (PIAS1) gene of Homo sapiens activated STAT, 1 deposited with Accession No. NM_{—}016166 in the database. From this study result, it was however found that the PIG23 protooncogene is highly expressed in various human tumors including the uterine cancer, while its expression is significantly reduced in various normal tissues.

A protein expressed from the protooncogene of the present invention contains 642 amino acids and has an amino acid sequence set forth in SEQ ID NO: 10 and a molecular weight of approximately 70 kDa.

4. PIG27

The protooncogene, a human proliferation-inducing gene 27 (PIG27), of the present invention (hereinafter, referred to as a PIG27 protooncogene) has a 446-bp full-length DNA sequence set forth in SEQ ID NO: 13.

In the DNA sequence of SEQ ID NO: 13, an open reading frame corresponding to nucleotide sequence positions from 20 to 337 (335-337: a stop codon) is a full-length protein coding region, and an amino acid sequence derived from the protein coding region is set forth in SEQ ID NO: 14 and contains 105 amino acids (hereinafter, referred to as “a PIG27 protein”).

The DNA sequence of SEQ ID NO: 13 has been deposited with Accession No. AY453399 in the GenBank database of U.S. National Institutes of Health (NIH) (Scheduled Release Date: Dec. 31, 2005), and the DNA base sequence result revealed that its DNA sequence was similar to that of the Homo sapiens DNAL4 full-length open reading frame (ORF) cDNA clone gene, etc. deposited with Accession No. CR456487 in the database. These genes have not been known in their function. From this study result, it was however found that the PIG27 protooncogene is highly expressed in various human tumors including the uterine cancer, while its expression is significantly reduced in various normal tissues.

A protein expressed from the protooncogene of the present invention contains 105 amino acids and has an amino acid sequence set forth in SEQ ID NO: 14 and a molecular weight of approximately 12 kDa.

5. PIG28

The protooncogene, a human proliferation-inducing gene 28 (PIG28), of the present invention (hereinafter, referred to as a PIG28 protooncogene) has a 1,024-bp full-length DNA sequence set forth in SEQ ID NO: 17.

In the DNA sequence of SEQ ID NO: 17, an open reading frame corresponding to nucleotide sequence positions from 33 to 998 (996-998: a stop codon) is a full-length protein coding region, and an amino acid sequence derived from the protein coding region is set forth in SEQ ID NO: 18 and contains 321 amino acids (hereinafter, referred to as “a PIG28 protein”).

The DNA sequence of SEQ ID NO: 17 has been deposited with Accession No. AY453398 in the GenBank database of U.S. National Institutes of Health (NIH) (Scheduled Release Date: Mar. 31, 2005), and the DNA base sequence result revealed that its DNA sequence was similar to that of the Homo sapiens annexin A4 (ANXA4) gene and the Homo sapiens annexin A4 gene, deposited with Accession No. NM_{—}001153 and BC000182 in the database, respectively. From this study result, it was however found that the PIG28 protooncogene is highly expressed in various human tumors including the uterine cancer, while its expression is significantly reduced in various normal tissues.

A protein expressed from the protooncogene of the present invention contains 321 amino acids and has an amino acid sequence set forth in SEQ ID NO: 18 and a molecular weight of approximately 36 kDa.

6. PIG30

The protooncogene, a human proliferation-inducing gene 30 (PIG30), of the present invention (hereinafter, referred to as a PIG30 protooncogene) has a 2,152-bp full-length DNA sequence set forth in SEQ ID NO: 21.

In the DNA sequence of SEQ ID NO: 21, an open reading frame corresponding to nucleotide sequence positions from 6 to 2,150 (2,148-2,150: a stop codon) is a full-length protein coding region, and an amino acid sequence derived from the protein coding region is set forth in SEQ ID NO: 22 and contains 714 amino acids (a PIG30 protein).

A protein expressed from the protooncogene of the present invention contains 714 amino acids and has an amino acid sequence set forth in SEQ ID NO: 22 and a molecular weight of approximately 82 kDa.

7. PIG31

The protooncogene, a human proliferation-inducing gene 31 (PIG31), of the present invention (hereinafter, referred to as a PIG31 protooncogene) has a 2,246-bp full-length DNA sequence set forth in SEQ ID NO: 25.

In the DNA sequence of SEQ ID NO: 25, an open reading frame corresponding to nucleotide sequence positions from 37 to 2,232 (2,230-2,232: a stop codon) is a full-length protein coding region, and an amino acid sequence derived from the protein coding region is set forth in SEQ ID NO: 26 and contains 731 amino acids (a PIG31 protein).

A protein expressed from the protooncogene of the present invention contains 731 amino acids and has an amino acid sequence set forth in SEQ ID NO: 26 and a molecular weight of approximately 83 kDa.

8. PIG38

The protooncogene, a human proliferation-inducing gene 38 (PIG38), of the present invention (hereinafter, referred to as a PIG38 protooncogene) has a 1,973-bp full-length DNA sequence set forth in SEQ ID NO: 29.

In the DNA sequence of SEQ ID NO: 29, an open reading frame corresponding to nucleotide sequence positions from 25 to 1,956 (1,954-1,956: a stop codon) is a full-length protein coding region, and an amino acid sequence derived from the protein coding region is set forth in SEQ ID NO: 30 and contains 643 amino acids (a PIG38 protein).

A protein expressed from the protooncogene of the present invention contains 643 amino acids and has an amino acid sequence set forth in SEQ ID NO: 30 and a molecular weight of approximately 73 kDa.

9. PIG40

The protooncogene, a human proliferation-inducing gene 40 (PIG40), of the present invention (hereinafter, referred to as a PIG40 protooncogene) has a 1,586-bp full-length DNA sequence set forth in SEQ ID NO: 33.

In the DNA sequence of SEQ ID NO: 33, an open reading frame corresponding to nucleotide sequence positions from 36 to 1,541 (1,539-1,541: a stop codon) is a full-length protein coding region, and an amino acid sequence derived from the protein coding region is set forth in SEQ ID NO: 34 and contains 501 amino acids (hereinafter, referred to as “a PIG40 protein”).

The DNA sequence of SEQ ID NO: 33 has been deposited with Accession No. AY762100 in the GenBank database of U.S. National Institutes of Health (NIH) (Scheduled Release Date: Dec. 31, 2005), and the DNA base sequence result revealed that its DNA sequence was similar to that of the Homo sapiens aspartyl-tRNA synthetase (DARS) gene, etc. deposited with Accession No. NM_{—}001349 in the database. From this study result, it was however found that the PIG40 protooncogene is highly expressed in various human tumors including the leukemia, while its expression is significantly reduced in various normal tissues.

A protein expressed from the protooncogene of the present invention contains 501 amino acids and has an amino acid sequence set forth in SEQ ID NO: 34 and a molecular weight of approximately 57 kDa.

10. PIG43

The protooncogene, a human proliferation-inducing gene 43 (PIG43), of the present invention (hereinafter, referred to as a PIG43 protooncogene) has a 1,245-bp full-length DNA sequence set forth in SEQ ID NO: 37.

In the DNA sequence of SEQ ID NO: 37, an open reading frame corresponding to nucleotide sequence positions from 57 to 758 (756-758: a stop codon) is a full-length protein coding region, and an amino acid sequence derived from the protein coding region is set forth in SEQ ID NO: 38 and contains 233 amino acids (hereinafter, referred to as “a PIG43 protein”).

A protein expressed from the protooncogene of the present invention contains 233 amino acids and has an amino acid sequence set forth in SEQ ID NO: 38 and a molecular weight of approximately 26 kDa. However, one or more amino acids may be substituted, added or deleted in the amino acid sequence of the protein within a range that does not affect functions of the protein, and only some of the protein may be used depending on its usage. Such a modified amino acid sequence is also included in the scope of the present invention. Accordingly, the present invention also includes a polypeptide having substantially the same amino acid sequence as the oncogenic protein; and fragments thereof. The term “substantially the same polypeptide” means a polypeptide having sequence homology of at least 80%, preferably at least 90%, and the most preferably at least 95%.

11. PIG44

The protooncogene, a human proliferation-inducing gene 44 (PIG44), of the present invention (hereinafter, referred to as a PIG44 protooncogene) has a 1,721-bp full-length DNA sequence set forth in SEQ ID NO: 41.

In the DNA sequence of SEQ ID NO: 41, an open reading frame corresponding to nucleotide sequence positions from 55 to 1,512 (1,510-1,512: a stop codon) is a full-length protein coding region, and an amino acid sequence derived from the protein coding region is set forth in SEQ ID NO: 42 and contains 485 amino acids (a PIG44 protein).

A protein expressed from the protooncogene of the present invention contains 485 amino acids and has an amino acid sequence set forth in SEQ ID NO: 42 and a molecular weight of approximately 55 kDa.

12. PIG46

The protooncogene, a human proliferation-inducing gene 46 (PIG46), of the present invention (hereinafter, referred to as a PIG46 protooncogene) has a 1,312-bp full-length DNA sequence set forth in SEQ ID NO: 45.

In the DNA sequence of SEQ ID NO: 45, an open reading frame corresponding to nucleotide sequence positions from 5 to 1,297 (1,295-1,297: a stop codon) is a full-length protein coding region, and an amino acid sequence derived from the protein coding region is set forth in SEQ ID NO: 46 and contains 430 amino acids (a PIG46 protein).

A protein expressed from the protooncogene of the present invention contains 430 amino acids and has an amino acid sequence set forth in SEQ ID NO: 46 and a molecular weight of approximately 48 kDa.

13. PIG47

The protooncogene, a human proliferation-inducing gene 47 (PIG47), of the present invention (hereinafter, referred to as a PIG47 protooncogene) has a 827-bp full-length DNA sequence set forth in SEQ ID NO: 49.

In the DNA sequence of SEQ ID NO: 49, an open reading frame corresponding to nucleotide sequence positions from 56 to 826 (824-826: a stop codon) is a full-length protein coding region, and an amino acid sequence derived from the protein coding region is set forth in SEQ ID NO: 50 and contains 256 amino acids (a PIG47 protein).

A protein expressed from the protooncogene of the present invention contains 256 amino acids and has an amino acid sequence set forth in SEQ ID NO: 50 and a molecular weight of approximately 29 kDa.

14. PIG48

The protooncogene, a human proliferation-inducing gene 48 (PIG48), of the present invention (hereinafter, referred to as a PIG48 protooncogene) has a 1,707-bp full-length DNA sequence set forth in SEQ ID NO: 53.

In the DNA sequence of SEQ ID NO: 53, an open reading frame corresponding to nucleotide sequence positions from 57 to 1,694 (1,692-1,694: a stop codon) is a full-length protein coding region, and an amino acid sequence derived from the protein coding region is set forth in SEQ ID NO: 54 and contains 545 amino acids (a PIG48 protein).

A protein expressed from the protooncogene of the present invention contains 545 amino acids and has an amino acid sequence set forth in SEQ ID NO: 54 and a molecular weight of approximately 60 kDa.

15. PIG50

The protooncogene, a human proliferation-inducing gene 50 (PIG50), of the present invention (hereinafter, referred to as a PIG50 protooncogene) has a 643-bp full-length DNA sequence set forth in SEQ ID NO: 57.

In the DNA sequence of SEQ ID NO: 57, an open reading frame corresponding to nucleotide sequence positions from 2 to 595 (593-595: a stop codon) is a full-length protein coding region, and an amino acid sequence derived from the protein coding region is set forth in SEQ ID NO: 58 and contains 197 amino acids (a PIG50 protein).

A protein expressed from the protooncogene of the present invention contains 197 amino acids and has an amino acid sequence set forth in SEQ ID NO: 58 and a molecular weight of approximately 22 kDa.

16. PIG54

The protooncogene, a human proliferation-inducing gene 54 (PIG54), of the present invention (hereinafter, referred to as a PIG54 protooncogene) has a 1,936-bp full-length DNA sequence set forth in SEQ ID NO: 61.

In the DNA sequence of SEQ ID NO: 61, an open reading frame corresponding to nucleotide sequence positions from 38 to 1,840 (1,838-1,840: a stop codon) is a full-length protein coding region, and an amino acid sequence derived from the protein coding region is set forth in SEQ ID NO: 62 and contains 600 amino acids (a PIG54 protein).

A protein expressed from the protooncogene of the present invention contains 600 amino acids and has an amino acid sequence set forth in SEQ ID NO: 62 and a molecular weight of approximately 69 kDa.

17. PIG55

The protooncogene, a human proliferation-inducing gene 55 (PIG55), of the present invention (hereinafter, referred to as a PIG55 protooncogene) has a 526-bp full-length DNA sequence set forth in SEQ ID NO: 65.

In the DNA sequence of SEQ ID NO: 65, an open reading frame corresponding to nucleotide sequence positions from 15 to 485 (483-485: a stop codon) is a full-length protein coding region, and an amino acid sequence derived from the protein coding region is set forth in SEQ ID NO: 66 and contains 156 amino acids (a PIG55 protein).

A protein expressed from the protooncogene of the present invention contains 156 amino acids and has an amino acid sequence set forth in SEQ ID NO: 66 and a molecular weight of approximately 18 kDa.

18. GIG9

The protooncogene, a human protooncogene GIG9, of the present invention has a 1,008-bp full-length DNA sequence set forth in SEQ ID NO: 69.

In the DNA sequence of SEQ ID NO: 69, an open reading frame corresponding to nucleotide sequence positions from 1 to 1,008 (1006-1008: a stop codon) is a full-length protein coding region, and an amino acid sequence derived from the protein coding region is set forth in SEQ ID NO: 70 and contains 335 amino acids (hereinafter, referred to as “a GIG9 protein”).

The DNA sequence of SEQ ID NO: 69 has been deposited with Accession No. AY453396 in the GenBank database of U.S. National Institutes of Health (NIH) (Scheduled Release Date: Mar. 31, 2005), and the DNA base sequence result revealed that its DNA sequence was similar to that of the Homo sapiens syntaxin 18 (STX18) gene deposited with Accession No. NM_{—}016930 in the database. From this study result, it was however found that the GIG9 protooncogene is highly expressed in various human tumors including the uterine cancer, while its expression is significantly reduced in various normal tissues.

A protein expressed from the protooncogene of the present invention contains 335 amino acids and has an amino acid sequence set forth in SEQ ID NO: 70 and a molecular weight of approximately 38 kDa.

19. HLC-9

The protooncogene, a human lung cancer-associated gene 9, of the present invention (hereinafter, referred to as an HLC9 protooncogene) has a 1,382-bp full-length DNA sequence set forth in SEQ ID NO: 73.

In the DNA sequence of SEQ ID NO: 73, an open reading frame corresponding to nucleotide sequence positions from 27 to 1,370 (1,368-1,370: a stop codon) is a full-length protein coding region, and an amino acid sequence derived from the protein coding region is set forth in SEQ ID NO: 74 and contains 447 amino acids (hereinafter, referred to as “an HLC9 protein”).

The DNA sequence of SEQ ID NO: 73 has been deposited with Accession No. AY189686 in the GenBank database of U.S. National Institutes of Health (NIH) (Scheduled Release Date: May 1, 2004), and the DNA base sequence result revealed that some of its DNA sequence was similar to that of the Homo sapiens protein phosphotase 2 (formerly 2A), regulatory subunit B (PR 52), α-isoform (PPP2R2A) gene deposited with Accession No. NM_{—}002717 in the database. From this study result, it was however found that the HLC9 protooncogene is highly expressed in various human tumors including the lung cancer, while its expression is significantly reduced in various normal tissues.

A protein expressed from the protooncogene HLC9 of the present invention contains 447 amino acids and has an amino acid sequence set forth in SEQ ID NO: 74 and a molecular weight of approximately 51 kDa.

20. GIG18

The protooncogene, a GIG18 gene, of the present invention (hereinafter, referred to as a GIG18 protooncogene) has a 1,301-bp full-length DNA sequence set forth in SEQ ID NO: 77.

In the DNA sequence of SEQ ID NO: 77, an open reading frame corresponding to nucleotide sequence positions from 3 to 1,244 (1,242-1,244: a stop codon) is a full-length protein coding region, and an amino acid sequence derived from the protein coding region is set forth in SEQ ID NO: 78 and contains 413 amino acids (a GIG18 protein).

A protein expressed from the protooncogene of the present invention contains 413 amino acids and has an amino acid sequence set forth in SEQ ID NO: 78 and a molecular weight of approximately 46 kDa.

21. MIG22

The protooncogene, a human migration-inducing gene 14 (MIG22), of the present invention (hereinafter, referred to as an MIG22 protooncogene) has a 749-bp full-length DNA sequence set forth in SEQ ID NO: 81.

In the DNA sequence of SEQ ID NO: 81, an open reading frame corresponding to nucleotide sequence positions from 15 to 734 (732-734: a stop codon) is a full-length protein coding region, and an amino acid sequence derived from the protein coding region is set forth in SEQ ID NO: 82 and contains 239 amino acids (hereinafter, referred to as “an MIG22 protein”).

The DNA sequence of SEQ ID NO: 81 has been deposited with Accession No. AY771595 in the GenBank database of U.S. National Institutes of Health (NIH) (Scheduled Release Date: Dec. 31, 2005), and the DNA base sequence result revealed that its DNA sequence was similar to that of the genes deposited with Accession No. D45248 and BC072025 in the database, respectively. Contrary to the functions of the RAE1 gene as reported previously, it was however found from this study result that the MIG22 protooncogene is highly expressed in various human tumors including the lung cancer, while its expression is significantly reduced in various normal tissues.

A protein expressed from the protooncogene of the present invention contains 239 amino acids and has an amino acid sequence set forth in SEQ ID NO: 82 and a molecular weight of approximately 27 kDa.

Meanwhile, because of degeneracy of codons, or considering preference of codons for living organisms to express the protooncogenes, the protooncogenes of the present invention may be variously modified in coding regions without changing an amino acid sequence of the oncogenic protein expressed from the coding region, and also be variously modified or changed in a region except the coding region within a range that does not affect the gene expression. Such a modified gene is also included in the scope of the present invention. Accordingly, the present invention also includes polynucleotides having substantially the same DNA sequences as the above-mentioned protooncogenes; and fragments thereof. The term “substantially the same polynucleotide” means a DNA sequence having a sequence homology of at least 80%, preferably at least 90%, and the most preferably at least 95%.

Also, one or more amino acids may be substituted, added or deleted even in the amino acid sequences of the proteins of the present invention within a range that does not affect functions of the proteins, and only some of the proteins may be used depending on their usage. Such a modified amino acid sequence is also included in the scope of the present invention. Accordingly, the present invention also includes polypeptides having substantially the same amino acid sequences as the oncogenic proteins; and fragments thereof. The term “substantially the same polypeptide” means a polypeptide having sequence homology of at least 80%, preferably at least 90%, and the most preferably at least 95%.

The protooncogenes and the proteins of the present invention may be separated from human cancer tissues, or be also synthesized according to the known methods for synthesizing DNA or peptide. Also, the genes prepared thus may be inserted into a vector for expression in the microorganisms, already known in the art, to obtain expression vectors, and then the expression vectors may be introduced into suitable host cells, for example Escherichia coli, yeast cells, etc. DNA of the genes of the present invention may be replicated in a large quantity or its protein may be produced in a commercial quantity in such a transformed host. For example, a transformant may be obtained in the present invention by inserting a PIG full-length cDNA into the expression vector pBAD/Thio-Topo (Invitrogen, U.S.), followed by transforming E. coli DH5 α with the resultant expression vector.

Upon constructing the expression vectors, expression regulatory sequences such as a promoter and a terminator, autonomously replicating sequences, secretion signals, etc. may be suitably selected and combined depending on kinds of the host cells that produce the protooncogenes or the proteins.

The genes of the present invention are proved to be strong oncogenes capable of developing the breast cancer since it was revealed the genes were hardly expressed in a normal breast tissue, but overexpressed in a breast cancer tissue and a breast cancer cell line in the analysis methods such as a northern blotting, etc. In addition to epithelial tissues such as the breast cancer, the protooncogenes of the present invention are highly expressed in other cancerous tumors such as breast cancer, leukemia, uterine cancer, malignant lymphoma, etc. Accordingly, the protooncogenes of the present invention are considered to be common oncogenes in the various oncogenesis, and may be effectively used for diagnosing the various cancers, producing the transformed animals and for anti-sense gene therapy, etc.

For example, a method for diagnosing the cancer using the protooncogenes includes a step of determining whether or not a subject has the protooncogenes of the present invention by detecting the protooncogenes using the various methods known in the art after all or some of the protooncogenes are used as proves to hybridize with nucleic acid extracted from the subject's body fluids. It can be easily confirmed that the genes are present in the tissue samples by using the probes labeled with a radioactive isotope, an enzyme, etc. Accordingly, the present invention provides kits for diagnosing the cancer including all or some of the protooncogenes.

The transformed animals may be obtained by introducing the protooncogenes of the present invention into mammals, for example rodents such as a rat, and the protooncogenes are preferably introduced at the fertilized egg stage prior to at least 8-cell stage. The transformed animals prepared thus may be effectively used for searching carcinogenic substances or anticancer substances such as antioxidants.

The proteins derived from the protooncogenes of the present invention may be effectively used as a diagnostic tool to produce antibodies. The antibodies of the present invention may be produced as the monoclonal or polyclonal antibodies according to the conventional methods known in the art using the proteins expressed from the protooncogenes of the present invention; or fragments thereof, wherein the proteins have amino acid sequences selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; SEQ ID NO: 58; SEQ ID NO: 62; SEQ ID NO: 66; SEQ ID NO: 70; SEQ ID NO: 74; SEQ ID NO: 78; and SEQ ID NO: 82. Therefore, such an antibody may be used to diagnose the cancer by determining whether or not the proteins are expressed in the body fluid samples of the subject using the method known in the art, for example an enzyme linked immunosorbent assay (ELISA), a radioimmunoassay (RIA), a sandwich assay, western blotting or immunoblotting on the polyacrylamide gel, etc.

Also, the protooncogenes of the present invention may be used to establish cancer cell lines that can grow in an uncontrolled manner, and such a cell line may be, for example, produced from the tumorous tissue developed in the back of a nude mouse using fibroblast cell transfected with the protooncogenes. This cancer cell line may be effectively used for searching anticancer agents, etc.

Hereinafter, the present invention will be described in detail referring to preferred examples, but the description proposed herein is just a preferable example for the purpose of illustrations only, not intended to limit the scope of the invention.

### BRIEF DESCRIPTION OF THE DRAWINGS

These and other features, aspects, and advantages of preferred embodiments of the present invention will be more fully described in the following detailed description, taken accompanying drawings. In the drawings:

FIGS. 1 to 21 are diagrams showing results of the differential display reverse transcription-polymerase chain reaction (DDRT-PCR) to determine whether or not FC21 (FIG. 1), FC23 (FIG. 6), FC34 (FIG. 7), FC24 (FIG. 12), FC54 (FIG. 13), FC71 (FIG. 14), BBCC5-5 (FIG. 15) and FC4 (FIG. 17) are expressed in a normal breast tissue, a breast cancer tissue and an MCF-7 cancer cell, respectively; whether or not an MC113 DNA fragment (FIG. 2), a CA338d DNA fragment (FIG. 3), an H124 DNA fragment (FIG. 4), an H122 DNA fragment (FIG. 5) and an H148 DNA fragment (FIG. 18) are expressed in a normal exocervical tissue, a cervical tumor tissue, a metastatic lymph node tumor tissue and a CUMC-6 cancer cell, respectively; whether or not HP103 (FIG. 8), HP11 (FIG. 10), HP23 (FIG. 11), HP15 (FIG. 16) and HP47 (FIG. 20) are expressed in a normal liver tissue, a liver cancer tissue and an HepG2 liver cancer cell, respectively; whether or not a GV11 DNA fragment (FIG. 9) is expressed in a normal peripheral blood leukocyte tissue, a leukemia tissue and a K562 leukemia cell line; and whether or not L738 (FIG. 19) and L690 (FIG. 21) are expressed in a normal lung tissue, a left lung cancer tissue, a metastatic lung cancer tissue metastasized from the left lung to the right lung, and an A549 lung cancer cell, respectively.

FIGS. 22 to 42 are diagrams showing northern blotting results to determine whether or not PIG12 (FIG. 22), PIG30 (FIG. 27), PIG31 (FIG. 28), PIG46 (FIG. 33), PIG47 (FIG. 34), PIG48 (FIG. 35), PIG50 (FIG. 36) and PIG55 (FIG. 38) protooncogenes are expressed in a breast cancer tissue, respectively; whether or not PIG18 (FIG. 23), PIG23 (FIG. 24), PIG27 (FIG. 25), PIG28 (FIG. 26) and GIG9 (FIG. 39) protooncogenes are expressed in a normal exocervical tissue, a uterine cancer tissue, a metastatic cervical lymph node tumor tissue and a cervical cancer cell line, respectively; whether or not PIG38 (FIG. 29); PIG43 (FIG. 31); PIG44 (FIG. 32); PIG54 (FIG. 37); GIG18 (FIG. 41) protooncogenes are expressed in a normal human liver, a liver cancer and a liver cancer cell line; whether or not a PIG40 (FIG. 30) protooncogene is expressed in a normal peripheral blood leukocyte tissue, a leukemia tissue and a K562 leukemia cell line; and whether or not HLC9 (FIG. 40) and MIG22 (FIG. 42) protooncogenes are expressed in a normal lung tissue, a left lung cancer tissue, a metastatic lung cancer tissue metastasized from the left lung to the right lung, and A549, NCI-H2009 and NCI-H441 lung cancer cell lines, respectively; and bottoms of FIGS. 22 to 42 are diagrams showing northern blotting results obtained by hybridizing the same samples as in the tops of FIGS. 22 to 42 with β-actin probe, respectively.

FIGS. 43 to 63 are diagrams showing northern blotting results to determine whether or not PIG12 (FIG. 43), PIG18 (FIG. 44), PIG23 (FIG. 45), PIG27 (FIG. 46), PIG28 (FIG. 47), PIG30 (FIG. 48), PIG31 (FIG. 49), PIG38 (FIG. 50), PIG40 (FIG. 51), PIG43 (FIG. 52), PIG44 (FIG. 53), PIG46 (FIG. 54), PIG47 (FIG. 55), PIG48 (FIG. 56), PIG50 (FIG. 57), PIG54 (FIG. 58), PIG55 (FIG. 59), GIG9 (FIG. 60), HLC-9 (FIG. 61), GIG18 (FIG. 62) and MIG22 (FIG. 63) protooncogenes are expressed in a normal human 12-lane multiple tissues, respectively; and bottoms of FIGS. 43 to 63 are diagrams showing northern blotting results obtained by hybridizing the same samples as in the tops of FIGS. 43 to 63 with β-actin probe, respectively.

FIGS. 64 to 84 are diagrams showing northern blotting results to determine whether or not PIG12 (FIG. 64), PIG18 (FIG. 65), PIG23 (FIG. 66), PIG27 (FIG. 67), PIG28 (FIG. 68), PIG30 (FIG. 69), PIG31 (FIG. 70), PIG38 (FIG. 71), PIG40 (FIG. 72), PIG43 (FIG. 73), PIG44 (FIG. 74), PIG46 (FIG. 75), PIG47 (FIG. 76), PIG48 (FIG. 77), PIG50 (FIG. 78), PIG54 (FIG. 79), PIG55 (FIG. 80), GIG9 (FIG. 81), HLC-9 (FIG. 82), GIG18 (FIG. 83) and MIG22 (FIG. 84) protooncogenes are expressed in human cancer cell lines, respectively; and bottoms of FIGS. 64 to 84 are diagrams showing northern blotting results obtained by hybridizing the same samples as in the tops of FIGS. 64 to 84 with β-actin probe, respectively.

FIGS. 85 to 105 are diagrams showing results of sodium dodecyl sulfate-polyacrylamide gel electrophoresis (SDS-PAGE) to determine sizes of the proteins expressed before and after L-arabinose induction after PIG12 (FIG. 85), PIG18 (FIG. 86), PIG23 (FIG. 87), PIG27 (FIG. 88), PIG28 (FIG. 89), PIG30 (FIG. 90), PIG31 (FIG. 91), PIG38 (FIG. 92), PIG40 (FIG. 93), PIG43 (FIG. 94), PIG44 (FIG. 95), PIG46 (FIG. 96), PIG47 (FIG. 97), PIG48 (FIG. 98), PIG50 (FIG. 99), PIG54 (FIG. 100), PIG55 (FIG. 101), GIG9 (FIG. 102), HLC-9 (FIG. 103), GIG18 (FIG. 104) and MIG22 (FIG. 105) protooncogenes of the present invention are transformed into Escherichia coli, respectively.

### BEST MODES FOR CARRYING OUT THE INVENTION

Hereinafter, preferred embodiments of the present invention will be described in detail with reference to the accompanying drawings.

### Example 1

### Cultivation of Tumor Cell and Separation of Total RNA

1-1. PIG12, PIG30, PIG31, PIG46, PIG47, PIG48, PIG50 and PIG55

(Step 1) Cultivation of Tumor Cell

In order to conduct the mRNA differential display method, a normal breast tissue sample was obtained from a breast cancer patient who has been subject to mastectomy, and a primary breast cancer tissue was obtained during mastectomy from a breast cancer patient who has not been subject to the anticancer chemotherapy and/or radiation therapy upon a surgical operation. MCF-7 (American Type Culture Collection; ATCC Number HTB-22) was used as the human breast cancer cell line in the differential display method. The culture cells used in this experiment are at the exponentially growing stage, and the cells showing a viability of at least 95% were used herein when a trypan blue dye is stained (see Freshney, “Culture of Animal Cells: A Manual of Basic Technique” 2nd Ed., A. R. Liss, New York, (1987)).

(Step 2) Separation of RNA and mRNA Differential Display Method

The total RNA samples were separated from the normal breast tissue, the primary breast cancer tissue and the MCF-7 cell, each obtained in Step 1, using the commercially available system RNeasy total RNA kit (Qiagen Inc., Germany). DNA contaminants were removed from the RNA samples using the message clean kit (GenHunter Corp., Brookline, Mass., U.S.).

1-2. PIG18, PIG23 PIG27, PIG28 and GIG9

(Step 1) Cultivation of Tumor Cell

In order to conduct the mRNA differential display method, a normal exocervical tissue was obtained from a patient suffering from a uterine myoma who has been subject to hysterectomy, and a primary cervical tumor tissue and a metastatic lymph node tumor tissue were obtained from a uterine cancer patient who has not been previously subject to the anticancer chemotherapy and/or radiation therapy upon a surgical operation. CUMC-6 (Kim, J. W. et al., Gynecol. Oncol. 62: 230-240, 1996) was used as the human cervical cancer cell line in the differential display method.

Cells obtained from the obtained tissues and the CUMC-6 cell line were grown in Waymouth's MB 752/1 media (Gibco) containing 2 mM glutamine, 100 IU/ml penicillin, 100 μg/ml streptomycin and 10% fetal bovine serum (Gibco, U.S.). The culture cells used in this experiment are at the exponentially growing stage, and the cells showing a viability of at least 95% in a trypan blue staining were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal exocervical tissue, the primary cervical tumor tissue, the metastatic lymph node tumor tissue and the CUMC-6 cell, each obtained in Step 1, using the commercially available system RNeasy total RNA kit (Qiagen Inc., Germany. DNA contaminants were removed from the RNA samples using the message clean kit (GenHunter Corp., Brookline, Mass., U.S.).

1-3. PIG38, PIG43, PIG44, PIG54 and GIG18

(Step 1) Cultivation of Tumor Cell

In order to conduct the mRNA differential display method, a normal liver tissue was obtained from a patient who has been subject to liver biopsy, and a primary liver tumor tissue was obtained from a liver cancer patient who has not been previously subject to the anticancer chemotherapy and/or radiation therapy during the liver biopsy. HepG2 (American Type Culture Collection) was used as the human liver cancer cell line in the differential display method. The culture cells used in this experiment are at the exponentially growing stage, and the cells showing a viability of at least 95% were used herein when a trypan blue dye is stained (see Freshney, “Culture of Animal Cells: A Manual of Basic Technique” 2nd Ed., A. R. Liss, New York, (1987)).

(Step 2) Separation of RNA and mRNA Differential Display Method

The total RNA samples were separated from the normal liver tissue, the primary liver cancer tissue and the HepG2 cell, each obtained in Step 1, using the commercially available system RNeasy total RNA kit (Qiagen Inc., Germany). DNA contaminants were removed from the RNA samples using the message clean kit (GenHunter Corp., Brookline, Mass., U.S.).

1-4. PIG40

(Step 1) Cultivation of Tumor Cell

In order to conduct the mRNA differential display method, a peripheral blood leukocyte tissue was obtained from a normal person, and a primary leukemic bone marrow tissue was obtained from a leukemia patient who has not been previously subject to the anticancer chemotherapy and/or radiation therapy during the bone marrow biopsy. K-562 (Americal Type Cell Collection; ATCC Number CCL-243) was used as the human chronic myelogenous leukemia cell line in the differential display method.

Cells obtained from the obtained tissues and the K-562 cell line were grown in Waymouth's MB 752/1 media (Gibco) containing 2 mM glutamine, 100 IU/ml penicillin, 100 μg/ml streptomycin and 10% fetal bovine serum (Gibco, U.S.). The culture cells used in this experiment are at the exponentially growing stage, and the cells showing a viability of at least 95% in a trypan blue staining were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the peripheral blood leukocyte tissue of the normal person, the primary leukemic bone marrow tissue and the human chronic myelogenous leukemia cell line, each obtained in Step 1, using the commercially available system RNeasy total RNA kit (Qiagen Inc., Germany). DNA contaminants were removed from the RNA samples using the message clean kit (GenHunter Corp., Brookline, Mass., U.S.).

1-5. HLC-9 and MIG22

(Step 1) Cultivation of Tumor Cell

In order to conduct the mRNA differential display method, a normal lung tissue was obtained from a normal person, and a primary leukemic lung cancer tissue and a cancer tissue metastasized to the right lung were obtained from a lung cancer patient who has not been previously subject to the anticancer chemotherapy and/or radiation therapy during the surgical operation. A549 (American Type Culture Collection; ATCC Number CCL-185) was used as the human lung cancer cell line in the differential display method.

Cells obtained from the obtained tissues and the A549 lung cancer cell line were grown in Waymouth's MB 752/1 media (Gibco) containing 2 mM glutamine, 100 IU/ml penicillin, 100 μg/ml streptomycin and 10% fetal bovine serum (Gibco, U.S.). The culture cells used in this experiment are at the exponentially growing stage, and the cells showing a viability of at least 95% were used herein when a trypan blue dye is stained (see Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal lung tissue, the primary lung cancer tissue, the metastasized lung cancer tissue and the A549 cell, each obtained in Step 1, using the commercially available system RNeasy total RNA kit (Qiagen Inc., Germany). DNA contaminants were removed from the RNA samples using the message clean kit (GenHunter Corp., Brookline, Mass., U.S.).

### Example 2

### Differential Display Reverse Transcription-Polymerase Chain Reaction (DDRT-PCR)

The differential display reverse transcription was carried out using a slightly modified reverse transcription-polymerase chain reaction (RT-PCR) proposed by Liang, P. and A. B. Pardee.

2-1. PIG12

At first, reverse transcription was conducted on 0.2 μg of the total RNA obtained in Step 1 of Example 1 using an anchored primer H-T11A (5′-AAGCTTTTTTTTTTTC-3′, RNAimage kit, Genhunter, Cor., MA, U.S.) of SEQ ID NO: 3 as the anchored oligo-dT primer.

Then, a PCR reaction was carried out in the presence of 0.5 mM [α-³⁵S] dATP (1200 Ci/mmole) using the same anchored primer and the primer H-AP21 (SEQ ID NO: 4) (5′-AAGCTTTCTCTGG-3′) out of the random 5′-13-mer primers (RNAimage primer sets 1-5) H-AP1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragment was dissolved in a 6% polyacrylamide sequencing gel, and then a position of a differentially expressed band was determined using autoradiography.

A 262-base pair (bp) band with FC21 cDNA (Base positions from 913 to 1,174 of SEQ ID NO: 1) was cut out from the dried gel. The extracted gel was heated for 15 minutes to elute the FC21 cDNA, and then the PCR reaction was repeated with the same primers under the same condition as described above to re-amplify the FC21 cDNA, except that [α-³⁵S]-labeled dATP (1200 Ci/mmole) and 20 μM dNTP were not used herein.

2-2. PIG18

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 7 was used as the anchored oligo-dT primer, and a primer H-AP11 (5′-AAGCTTCGGGTAA-3′) having a DNA sequence set forth in SEQ ID NO: 8 was used herein.

A 277-base pair (bp) band with MC113 cDNA (Base positions from 2,023 to 2,299 of SEQ ID NO: 5) was cut out from the dried gel. The extracted gel was heated for 15 minutes to elute the MC113 cDNA, and then the PCR reaction was repeated with the same primers under the same condition as described above to re-amplify the MC113 cDNA, except that [α-³⁵S]-labeled dATP (1200 Ci/mmole) and 20 μM dNTP were not used herein.

2-3. PIG23

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 11 was used as the anchored oligo-dT primer, and a primer H-AP33 (5′-AAGCTTGCTGCTC-3′) having a DNA sequence set forth in SEQ ID NO: 12 was used herein.

A 278-base pair (bp) band with CA338d cDNA (Base positions from 1,822 to 2,099 of SEQ ID NO: 9) was cut out from the dried gel. The extracted gel was heated for 15 minutes to elute the CA338d cDNA, and then the PCR reaction was repeated with the same primers under the same condition as described above to re-amplify the CA338d cDNA, except that [α-³⁵S]-labeled dATP (1200 Ci/mmole) and 20 μM dNTP were not used herein.

2-4. PIG27

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 15 was used as the oligo-dT primer, and a primer H-AP12 (5′-AAGCTTGAGTGCT-3′) having a DNA sequence set forth in SEQ ID NO: 16 was used herein.

A 177-base pair (bp) band with H124 cDNA (Base positions from 243 to 419 of SEQ ID NO: 13) was cut out from the dried gel. The extracted gel was heated for 15 minutes to elute the H124 cDNA, and then the PCR reaction was repeated with the same primers under the same condition as described above to re-amplify the H124 cDNA, except that [α-³⁵S]-labeled dATP (1200 Ci/mmole) and 20 μM dNTP were not used herein.

2-5. PIG28

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 19 was used as the anchored oligo-dT primer, and a primer H-AP12 (5′-AAGCTTGAGTGCT-3′) having a DNA sequence set forth in SEQ ID NO: 20 was used herein.

A 232-base pair (bp) band with H122 cDNA (Base positions from 748 to 979 of SEQ ID NO: 17) was cut out from the dried gel. The extracted gel was heated for 15 minutes to elute the H122 cDNA, and then the PCR reaction was repeated with the same primers under the same condition as described above to re-amplify the H122 cDNA, except that [α-³⁵]-labeled dATP (1200 Ci/mmole) and 20 μM dNTP were not used herein.

2-6. PIG30

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 23 was used as the anchored oligo-dT primer, and a primer H-AP33 (5′-AAGCTTGCTGCTC-3′) having a DNA sequence set forth in SEQ ID NO: 24 was used herein.

A 271-base pair (bp) band with FC23 cDNA (Base positions from 1,823 to 2,093 of SEQ ID NO: 21) was cut out from the dried gel. The extracted gel was heated for 15 minutes to elute the FC23 cDNA, and then the PCR reaction was repeated with the same primers under the same condition as described above to re-amplify the FC23 cDNA, except that [α-³⁵S]-labeled dATP (1200 Ci/mmole) and 20 μM dNTP were not used herein.

2-7. PIG31

The PCR reaction was repeated in the same manner as in Example 2-1, except that an anchored primer H-T11G (5′-AAGCTTTTTTTTTTTG-3′) having a DNA sequence set forth in SEQ ID NO: 27 was used as the anchored oligo-dT primer, and a primer H-AP34 (5′-AAGCTTCAGCAGC-3′) having a DNA sequence set forth in SEQ ID NO: 28 was used herein.

A 312-base pair (bp) band with FC34 cDNA (Base positions from 1,884 to 2,195 of SEQ ID NO: 25) was cut out from the dried gel. The extracted gel was heated for 15 minutes to elute the FC34 cDNA, and then the PCR reaction was repeated with the same primers under the same condition as described above to re-amplify the FC34 cDNA, except that [α-³⁵S]-labeled dATP (1200 Ci/mmole) and 20 μM dNTP were not used herein.

2-8. PIG38

The PCR reaction was repeated in the same manner as in Example 2-1, except that an anchored primer H-T11C (5′-AAGCTTTTTTTTTTTC-3′) having a DNA sequence set forth in SEQ ID NO: 31 was used as the anchored oligo-dT primer, and a primer H-AP10 (5′-AAGCTTCCACGTA-3′) having a DNA sequence set forth in SEQ ID NO: 32 was used herein.

A 267-base pair (bp) band with HP103 cDNA (Base positions from 1,633 to 1,899 of SEQ ID NO: 29) was cut out from the dried gel. The extracted gel was heated for 15 minutes to elute the HP103 cDNA, and then the PCR reaction was repeated with the same primers under the same condition as described above to re-amplify the HP103 cDNA, except that [α-³⁵S]-labeled dATP (1200 Ci/mmole) and 20 μM dNTP were not used herein.

2-9. PIG40

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 35 was used as the anchored oligo-dT primer, and a primer H-AP11 (5′-AAGCTTCGGGTAA-3′) having a DNA sequence set forth in SEQ ID NO: 36 was used herein.

A 215-base pair (bp) band with GV11 cDNA (Base positions from 1,313 to 1,527 of SEQ ID NO: 33) was cut out from the dried gel. The extracted gel was heated for 15 minutes to elute the GV11 cDNA, and then the PCR reaction was repeated with the same primers under the same condition as described above to re-amplify the GV11 cDNA, except that [α-³⁵S]-labeled dATP (1200 Ci/mmole) and 20 μM dNTP were not used herein.

2-10. PIG43

The PCR reaction was repeated in the same manner as in Example 2-1, except that an anchored primer H-T11G (5′-AAGCTTTTTTTTTTTG-3′) having a DNA sequence set forth in SEQ ID NO: 39 was used as the anchored oligo-dT primer, and a primer H-AP11 (5′-AAGCTTCGGGTAA-3′) having a DNA sequence set forth in SEQ ID NO: 40 was used herein.

A 321-base pair (bp) band with HP11 cDNA (Base positions from 879 to 1,199 of SEQ ID NO: 37) was cut out from the dried gel. The extracted gel was heated for 15 minutes to elute the HP11 cDNA, and then the PCR reaction was repeated with the same primers under the same condition as described above to re-amplify the HP11 cDNA, except that [α-³⁵S]-labeled dATP (1200 Ci/mmole) and 20 μM dNTP were not used herein.

2-11. PIG44

The PCR reaction was repeated in the same manner as in Example 2-1, except that an anchored primer H-T11C (5′-AAGCTTTTTTTTTTTC-3′) having a DNA sequence set forth in SEQ ID NO: 43 was used as the anchored oligo-dT primer, and a primer H-AP23 (5′-AAGCTTGGCTATG-3′) having a DNA sequence set forth in SEQ ID NO: 44 was used herein.

A 311-base pair (bp) band with HP23 cDNA (Base positions from 1,633 to 1,899 of SEQ ID NO: 41) was cut out from the dried gel. The extracted gel was heated for 15 minutes to elute the HP23 cDNA, and then the PCR reaction was repeated with the same primers under the same condition as described above to re-amplify the HP23 cDNA, except that [α-³⁵S]-labeled dATP (1200 Ci/mmole) and 20 μM dNTP were not used herein.

2-12. PIG46

The PCR reaction was repeated in the same manner as in Example 2-1, except that an anchored primer H-T11A (5′-AAGCTTTTTTTTTTTA-3′) having a DNA sequence set forth in SEQ ID NO: 47 was used as the anchored oligo-dT primer, and a primer H-AP24 (5′-AAGCTTCACTAGC-3′) having a DNA sequence set forth in SEQ ID NO: 48 was used herein.

A 256-base pair (bp) band with FC24 cDNA (Base positions from 992 to 1,247 of SEQ ID NO: 45) was cut out from the dried gel. The extracted gel was heated for 15 minutes to elute the FC24 cDNA, and then the PCR reaction was repeated with the same primers under the same condition as described above to re-amplify the FC24 cDNA, except that [α-³⁵S]-labeled dATP (1200 Ci/mmole) and 20 μM dNTP were not used herein.

2-13. PIG47

The PCR reaction was repeated in the same manner as in Example 2-1, except that an anchored primer H-T11C (5′-AAGCTTTTTTTTTTTC-3′) having a DNA sequence set forth in SEQ ID NO: 51 was used as the anchored oligo-dT primer, and a primer H-AP5 (5′-AAGCTTAGTAGGC-3′) having a DNA sequence set forth in SEQ ID NO: 52 was used herein.

A 192-base pair (bp) band with FC54 cDNA (Base positions from 587 to 778 of SEQ ID NO: 49) was cut out from the dried gel. The extracted gel was heated for 15 minutes to elute the FC54 cDNA, and then the PCR reaction was repeated with the same primers under the same condition as described above to re-amplify the FC54 cDNA, except that [α-³⁵S]-labeled dATP (1200 Ci/mmole) and 20 μM dNTP were not used herein.

2-14. PIG48

The PCR reaction was repeated in the same manner as in Example 2-1, except that an anchored primer H-T11A (5′-AAGCTTTTTTTTTTTA-3′) having a DNA sequence set forth in SEQ ID NO: 55 was used as the anchored oligo-dT primer, and a primer H-AP7 (5′-AAGCTTAACGAGG-3′) having a DNA sequence set forth in SEQ ID NO: 56 was used herein.

A 272-base pair (bp) band with FC71 cDNA (Base positions from 1,348 to 1,619 of SEQ ID NO: 53) was cut out from the dried gel. The extracted gel was heated for 15 minutes to elute the FC71 cDNA, and then the PCR reaction was repeated with the same primers under the same condition as described above to re-amplify the FC71 cDNA, except that [α-³⁵S]-labeled dATP (1200 Ci/mmole) and 20 μM dNTP were not used herein.

2-15. PIG50

The PCR reaction was repeated in the same manner as in Example 2-1, except that an anchored primer H-T11C (5′-AAGCTTTTTTTTTTTC-3′) having a DNA sequence set forth in SEQ ID NO: 59 was used as the anchored oligo-dT primer, and a primer H-AP5 (5′-AAGCTTAGTAGGC-3′) having a DNA sequence set forth in SEQ ID NO: 60 was used herein.

A 182-base pair (bp) band with BBCC5-5 cDNA (Base positions from 418 to 599 of SEQ ID NO: 57) was cut out from the dried gel. The extracted gel was heated for 15 minutes to elute the BBCC5-5 cDNA, and then the PCR reaction was repeated with the same primers under the same condition as described above to re-amplify the BBCC5-5 cDNA, except that [α-³⁵S]-labeled dATP (1200 Ci/mmole) and 20 μM dNTP were not used herein.

2-16. PIG54

The PCR reaction was repeated in the same manner as in Example 2-1, except that an anchored primer H-T11A (5′-AAGCTTTTTTTTTTTA-3′) having a DNA sequence set forth in SEQ ID NO: 63 was used as the anchored oligo-dT primer, and a primer H-AP15 (5′-AAGCTTACGCAAC-3′) having a DNA sequence set forth in SEQ ID NO: 64 was used herein.

A 345-base pair (bp) band with HP15 cDNA (Base positions from 1,533 to 1,877 of SEQ ID NO: 61) was cut out from the dried gel. The extracted gel was heated for 15 minutes to elute the HP15 cDNA, and then the PCR reaction was repeated with the same primers under the same condition as described above to re-amplify the HP15 cDNA, except that [α-³⁵S]-labeled dATP (1200 Ci/mmole) and 20 μM dNTP were not used herein.

2-17. PIG55

The PCR reaction was repeated in the same manner as in Example 2-1, except that an anchored primer H-T11G (5′-AAGCTTTTTTTTTTTG-3′) having a DNA sequence set forth in SEQ ID NO: 67 was used as the anchored oligo-dT primer, and a primer H-AP4 (5′-AAGCTTCTCAACG-3′) having a DNA sequence set forth in SEQ ID NO: 68 was used herein.

A 186-base pair (bp) band with FC4 cDNA (Base positions from 292 to 477 of SEQ ID NO: 65) was cut out from the dried gel. The extracted gel was heated for 15 minutes to elute the FC4 cDNA, and then the PCR reaction was repeated with the same primers under the same condition as described above to re-amplify the FC4 cDNA, except that [α-³⁵S]-labeled dATP (1200 Ci/mmole) and 20 μM dNTP were not used herein.

2-18. GIG9

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 71 was used as the anchored oligo-dT primer, and a primer H-AP14 (5′-AAGCTTGGAGCTT-3′) having a DNA sequence set forth in SEQ ID NO: 72 was used herein.

A 221-base pair (bp) band with H148 cDNA (Base positions from 769 to 989 of SEQ ID NO: 69) was cut out from the dried gel. The extracted gel was heated for 15 minutes to elute the H148 cDNA, and then the PCR reaction was repeated with the same primers under the same condition as described above to re-amplify the H148 cDNA, except that [α-³⁵S]-labeled dATP (1200 Ci/mmole) and 20 μM dNTP were not used herein.

2-19. HLC-9

The PCR reaction was repeated in the same manner as in Example 2-1, except that an anchored primer H-T11G (5′-AAGCTTTTTTTTTTTG-3′) having a DNA sequence set forth in SEQ ID NO: 75 was used as the anchored oligo-dT primer, and a primer H-AP7 (5′-AAGCTTAACGAGG-3′) having a DNA sequence set forth in SEQ ID NO: 76 was used herein.

A 322-base pair (bp) band with L738 cDNA (Base positions from 1,007 to 1,328 of SEQ ID NO: 73) was cut out from the dried gel. The extracted gel was heated for 15 minutes to elute the L738 cDNA, and then the PCR reaction was repeated with the same primers under the same condition as described above to re-amplify the L738 cDNA, except that [α-³⁵S]-labeled dATP (1200 Ci/mmole) and 20 μM dNTP were not used herein.

2-20. GIG18

The PCR reaction was repeated in the same manner as in Example 2-1, except that an anchored primer H-T11C (5′-AAGCTTTTTTTTTTTC-3′) having a DNA sequence set forth in SEQ ID NO: 79 was used as the anchored oligo-dT primer, and a primer H-AP4 (5′-AAGCTTCTCAACG-3′) having a DNA sequence set forth in SEQ ID NO: 80 was used herein.

A 321-base pair (bp) band with HP47 cDNA (Base positions from 879 to 1,199 of SEQ ID NO: 77) was cut out from the dried gel. The extracted gel was heated for 15 minutes to elute the HP47 cDNA, and then the PCR reaction was repeated with the same primers under the same condition as described above to re-amplify the HP47 cDNA, except that [α-³⁵S]-labeled dATP (1200 Ci/mmole) and 20 μM dNTP were not used herein.

2-21. MIG22

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 83 was used as the anchored oligo-dT primer, and a primer H-AP6 (5′-AAGCTTGCACCAT-3′) having a DNA sequence set forth in SEQ ID NO: 84 was used herein.

A 327-base pair (bp) band with L690 cDNA (Base positions from 273 to 599 of SEQ ID NO: 81) was cut out from the dried gel. The extracted gel was heated for 15 minutes to elute the L690 cDNA, and then the PCR reaction was repeated with the same primers under the same condition as described above to re-amplify the L690 cDNA, except that [α-³⁵S]-labeled dATP (1200 Ci/mmole) and 20 μM dNTP were not used herein.

### Example 3

### Cloning

The FC21 product; the MC113 product; the CA338d product; the H124 product; H122 product; the FC23 product; the FC34 product; the HP103 product; the GV11 product; the HP11 product; the HP23 product; the FC24 product; the FC54 product; the FC71 product; the BBCC5-5 product; the HP15 product; the FC4 product; the H148 product; the L738 product; the HP47 product; and the L690 PCR product, which were all re-amplified as described above, were inserted into a pGEM-T EASY vector, respectively, according to the manufacturer's manual using the TA cloning system (Promega, U.S.).

(Step 1) Ligation Reaction

2 μl of each of the FC21 product; the MC113 product; the CA338d product; the H124 product; H122 product; the FC23 product; the FC34 product; the HP103 product; the GV11 product; the HP11 product; the HP23 product; the FC24 product; the FC54 product; the FC71 product; the BBCC5-5 product; the HP15 product; the FC4 product; the H148 product; the L738 product; the HP47 product; and the L690 PCR product which were all re-amplified in Example 2, 1 μl of pGEM-T EASY vector (50 ng), 1 μl of T4 DNA ligase buffer (10×) and 1 μl of T4 DNA ligase (3 weiss units/μl; Promega) were put into a 0.5 ml test tube, and distilled water was added thereto to a final volume of 10 μl. The ligation reaction mixtures were incubated overnight at 14° C.

(Step 2) Transformation of TA Clone

E. coli JM109 (Promega, Wis., U.S.) was incubated in 10 ml of LB broth (10 g of bacto-tryptone, 5 g of bacto-yeast extract, 5 g of NaCl) until the optical density at 600 nm reached approximately 0.3 to 0.6. The incubated mixture was kept in ice for about 10 minutes and centrifuged at 4,000 rpm for 10 minutes at 4° C., and then the supernatant wad discarded and the cell was collected. The collected cell pellet was exposed to 10 ml of 0.1 M ice-cold CaCl₂ for approximately 30 minutes to 1 hours to produce a competent cell. The product was centrifuged again at 4,000 rpm for 10 minutes at 4° C., and then the supernatant wad discarded and the cell was collected and suspended in 2 ml of 0.1 M ice-cold CaCl₂.

200 μl of the competent cell suspension was transferred to a new microfuge tube, and 2 μl of each of the ligation reaction products prepared in Step 1 was added thereto. The resultant mixtures were incubated in a water bath at 42° C. for 90 seconds, and then quenched at 0° C. 800 μl of SOC medium (2.0 g of bacto-tryptone, 0.5 g of bacto-yeast extract, 1 ml of 1 M NaCl, 0.25 ml of 1 M KCl, 97 ml of TDW, 1 ml of 2 M Mg²⁺, 1 ml of 2 M glucose) was added thereto and the resultant mixtures were incubated at 37° C. for 45 minutes in a rotary shaking incubator at 220 rpm.

25 μl of X-gal (stored in 40 mg/ml of dimethylformamide) was spread with a glass rod on LB plates which were supplemented with ampicillin and previously maintained in the incubator at 37° C., and then 25 μl of each of the transformed cells was added thereto and spread again with a glass rod, and then incubated overnight at 37° C. After incubation, the 3 to 4 formed white colonies was selected and then each of the selected cells was seed-cultured in a LB plate which was supplemented with ampicillin. In order to construct plasmids, the strains proved to be colonies into which the ligation reaction products were introduced amongst the above colonies respectively, namely the transformed E. coli strains JM109/FC21; JM109/MC113; JM109/CA338d; JM109/H124; JM109/H122; JM109/FC23; JM109/FC34; JM109/HP103; JM109/GV11; JM109/HP11; JM109/HP23; JM109/FC24; JM109/FC54; JM109/FC71; JM109/BBCC5-5; JM109/HP15; JM109/FC4; JM109/H148; JM109/L738; JM109/HP47; and JM109/L690 were selected and incubated in 10 ml of terrific broth (900 ml of TDW, 12 g of bacto-tryptone, 24 g of bacto-yeast extract, 4 ml of glycerol, 0.17 M KH₂PO₄, 100 ml of 0.72 N K₂HPO₄).

### Example 4

### Separation of Recombinant Plasmid DNA

The FC21 plasmid DNA was separated from the transformed E. coli strain using a Wizard™ Plus Minipreps DNA purification kit (Promega, U.S.) according to the manufacturer's manual.

It was confirmed that a small amount of each of the separated plasmid DNAs was treated with a restriction enzyme ECoRI, and then electrophoresized in a 2% gel to confirm that the partial sequences of FC21; MC113; CA338d; H124; H122; FC23; FC34; HP103; GV11; HP11; HP23; FC24; FC54; FC71; BBCC5-5; HP15; FC4; H148; L738; HP47; and L690 were inserted into the plasmids, respectively.

### Example 5

### DNA Base Sequence Analysis

The FC21 product; the MC113 product; the CA338d product; the H124 product; H122 product; the FC23 product; the FC34 product; the HP103 product; the GV11 product; the HP11 product; the HP23 product; the FC24 product; the FC54 product; the FC71 product; the BBCC5-5 product; the HP15 product; the FC4 product; the H148 product; the L738 product; the HP47 product; and the L690 PCR product, all obtained in Example 2, were amplified, cloned, and then re-amplified according to the conventional method. The resultant fragments of FC21; MC113; CA338d; H124; H122; FC23; FC34; HP103; GV11; HP11; HP23; FC24; FC54; FC71; BBCC5-5; HP15; FC4; H148; L738; HP47; and L690 were sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 913 to 1,174 of SEQ ID NO: 1, which was designated “FC21” in the present invention.

The 262-bp cDNA fragment obtained above, namely FC21, was subject to the differential display reverse transcription-polymerase chain reaction (DDRT-PCR) using a 5′-random primer H-AP21 and a 3′-anchored primer H-T11A, and then confirmed using the electrophoresis. As shown in FIG. 1, it was revealed from the differential display (DD) that the gene was differentially expressed in the normal breast tissue, the breast cancer tissue and the MCF-7 cell. As seen in FIG. 1, the 262-bp cDNA fragment FC21 was highly expressed in the breast cancer and the MCF-7 cancer cell, but very rarely expressed in the normal tissue.

The DNA sequence of the said gene corresponds to nucleotide sequence positions from 2,023 to 2,299 of SEQ ID NO: 5, which was designated “MC113” in the present invention.

The 277-bp cDNA fragment obtained above, namely MC113, was subject to the differential display reverse transcription-polymerase chain reaction (DDRT-PCR) using a 5′-random primer H-AP11 and a 3′-anchored primer H-T11C, and then confirmed using the electrophoresis.

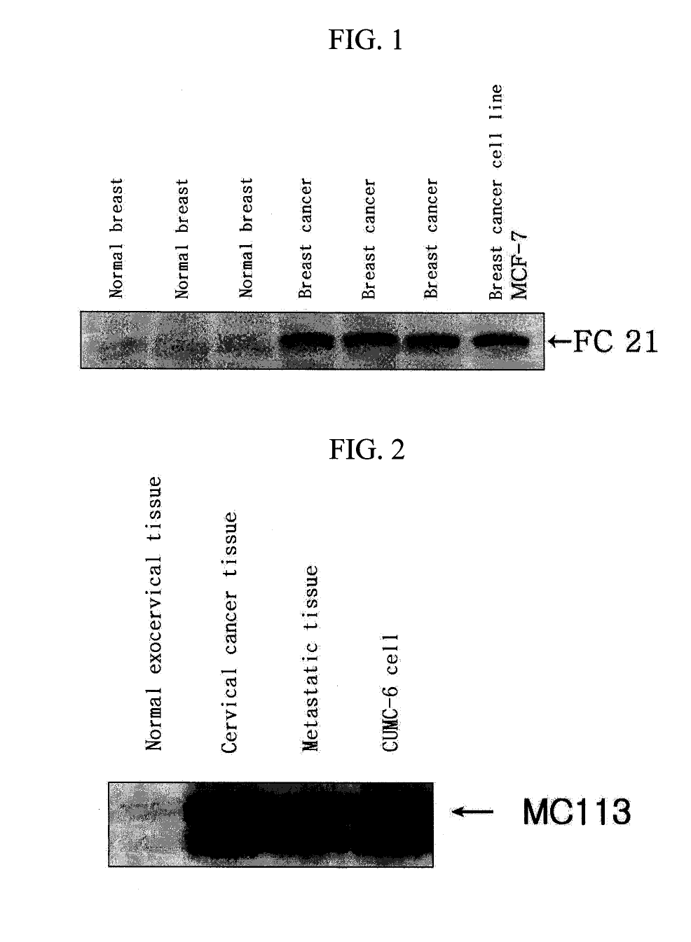

As shown in FIG. 2, it was revealed from the differential display (DD) that the gene was differentially expressed in the normal exocervical tissue, the metastatic lymph node tissue and the CUMC-6 cell. As seen in FIG. 2, the 277-bp cDNA fragment MC113 was expressed in the cervical cancer, the metastatic lymph node tissue and the CUMC-6 cancer cell, but rarely expressed in the normal tissue.

The DNA sequence of the said gene corresponds to nucleotide sequence positions from 1,822 to 2,099 of SEQ ID NO: 9, which was designated “CA338d” in the present invention.

The 278-bp cDNA fragment obtained above, namely CA338d, was subject to the differential display reverse transcription-polymerase chain reaction (DDRT-PCR) using a 5′-random primer H-AP33 and a 3′-anchored primer H-T11C, and then confirmed using the electrophoresis.

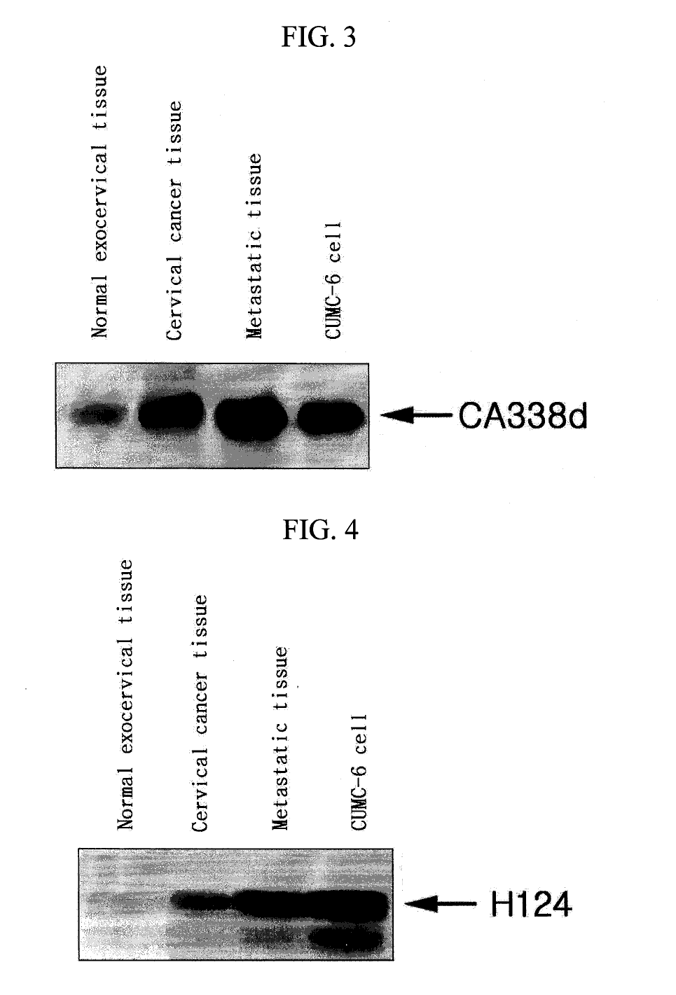

As shown in FIG. 3, it was revealed from the differential display (DD) that the gene was differentially expressed in the normal exocervical tissue, the metastatic lymph node tissue and the CUMC-6 cell. As seen in FIG. 3, the 278-bp cDNA fragment CA338d was expressed in the cervical cancer, the metastatic lymph node tissue and the CUMC-6 cancer cell, but very rarely expressed in the normal tissue.

The DNA sequence of the said gene corresponds to nucleotide sequence positions from 243 to 419 of SEQ ID NO: 13, which was designated “H124” in the present invention.

The 177-bp cDNA fragment obtained above, namely H124, was subject to the differential display reverse transcription-polymerase chain reaction (DDRT-PCR) using a 5′-random primer H-AP12 and a 3′-anchored primer H-TllA, and then confirmed using the electrophoresis.

As shown in FIG. 4, it was revealed from the differential display (DD) that the gene was differentially expressed in the normal exocervical tissue, the metastatic lymph node tissue and the CUMC-6 cell. As seen in FIG. 4, the 177-bp cDNA fragment H124 was expressed in the cervical cancer, the metastatic lymph node tissue and the CUMC-6 cancer cell, but very rarely expressed in the normal tissue.

The DNA sequence of the said gene corresponds to nucleotide sequence positions from 748 to 979 of SEQ ID NO: 17, which was designated “H122” in the present invention.

The 232-bp cDNA fragment obtained above, namely H122, was subject to the differential display reverse transcription-polymerase chain reaction (DDRT-PCR) using a 5′-random primer H-AP12 and a 3′-anchored primer H-T11C, and then confirmed using the electrophoresis.

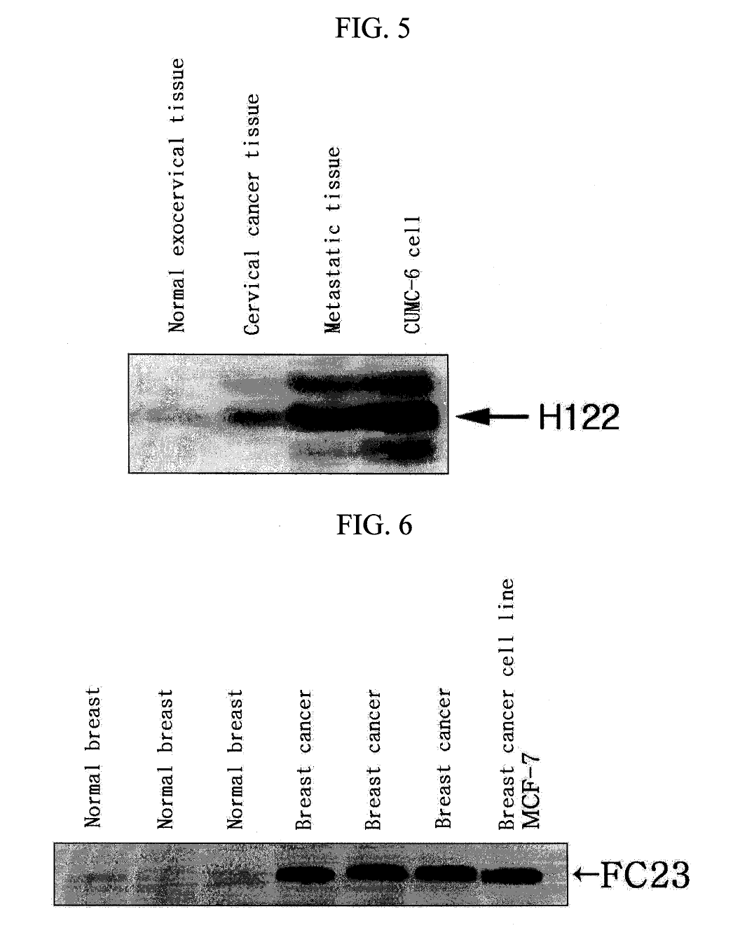

As shown in FIG. 5, it was revealed from the differential display (DD) that the gene was differentially expressed in the normal exocervical tissue, the metastatic lymph node tissue and the CUMC-6 cell. As seen in FIG. 5, the 232-bp cDNA fragment H122 was expressed in the cervical cancer, the metastatic lymph node tissue and the CUMC-6 cancer cell, but very rarely expressed in the normal tissue.

The DNA sequence of the said gene corresponds to nucleotide sequence positions from 1,823 to 2,093 of SEQ ID NO: 21, which was designated “FC23” in the present invention.

The 271-bp cDNA fragment obtained above, namely FC23, was subject to the differential display reverse transcription-polymerase chain reaction (DDRT-PCR) using a 5′-random primer H-AP23 and a 3′-anchored primer H-T11C, and then confirmed using the electrophoresis. As shown in FIG. 6, it was revealed from the differential display (DD) that the gene was differentially expressed in the normal breast tissue, the breast cancer tissue and the MCF-7 cell. As seen in FIG. 6, the 271-bp cDNA fragment FC23 was highly expressed in the breast cancer and the MCF-7 cancer cell, but very rarely expressed in the normal tissue.

The DNA sequence of the said gene corresponds to nucleotide sequence positions from 1,884 to 2,195 of SEQ ID NO: 25, which was designated “FC34” in the present invention.

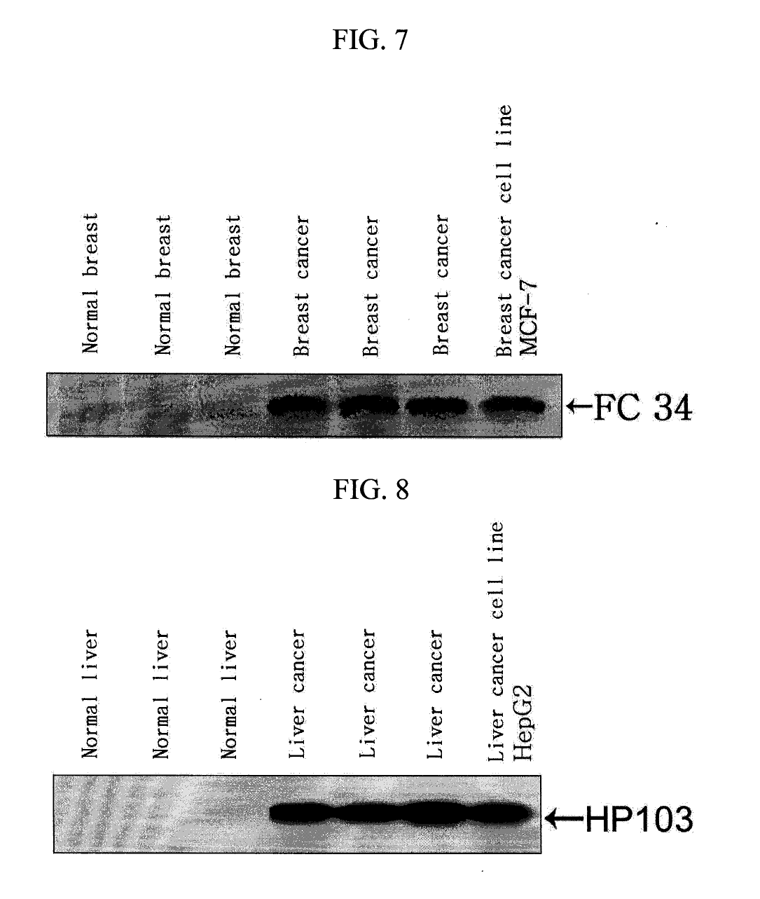

The 312-bp cDNA fragment obtained above, namely FC34, was subject to the differential display reverse transcription-polymerase chain reaction (DDRT-PCR) using a 5′-random primer H-AP34 and a 3′-anchored primer H-T11G, and then confirmed using the electrophoresis. As shown in FIG. 7, it was revealed from the differential display (DD) that the gene was differentially expressed in the normal breast tissue, the breast cancer tissue and the MCF-7 cell. As seen in FIG. 7, the 312-bp cDNA fragment FC34 was highly expressed in the breast cancer and the MCF-7 cancer cell, but very rarely expressed in the normal tissue.

The DNA sequence of the said gene corresponds to nucleotide sequence positions from 1,633 to 1,899 of SEQ ID NO: 29, which was designated “HP103” in the present invention.

The 267-bp cDNA fragment obtained above, namely HP103, was subject to the differential display reverse transcription-polymerase chain reaction (DDRT-PCR) using a 5′-random primer H-AP10 and a 3′-anchored primer H-T11C, and then confirmed using the electrophoresis. As shown in FIG. 8, it was revealed from the differential display (DD) that the gene was differentially expressed in the normal liver tissue, the liver cancer tissue and the HepG2 cell. As seen in FIG. 8, the 267-bp cDNA fragment HP103 was expressed in the liver cancer and the HepG2 cancer cell, but not expressed or detected in the normal tissue.

The DNA sequence of the said gene corresponds to nucleotide sequence positions from 1,313 to 1,527 of SEQ ID NO: 33, which was designated “GV1” in the present invention.

The 215-bp cDNA fragment obtained above, namely GV11, was subject to the differential display reverse transcription-polymerase chain reaction (DDRT-PCR) using a 5′-random primer H-AP11 and a 3′-anchored primer H-T11C, and then confirmed using the electrophoresis.

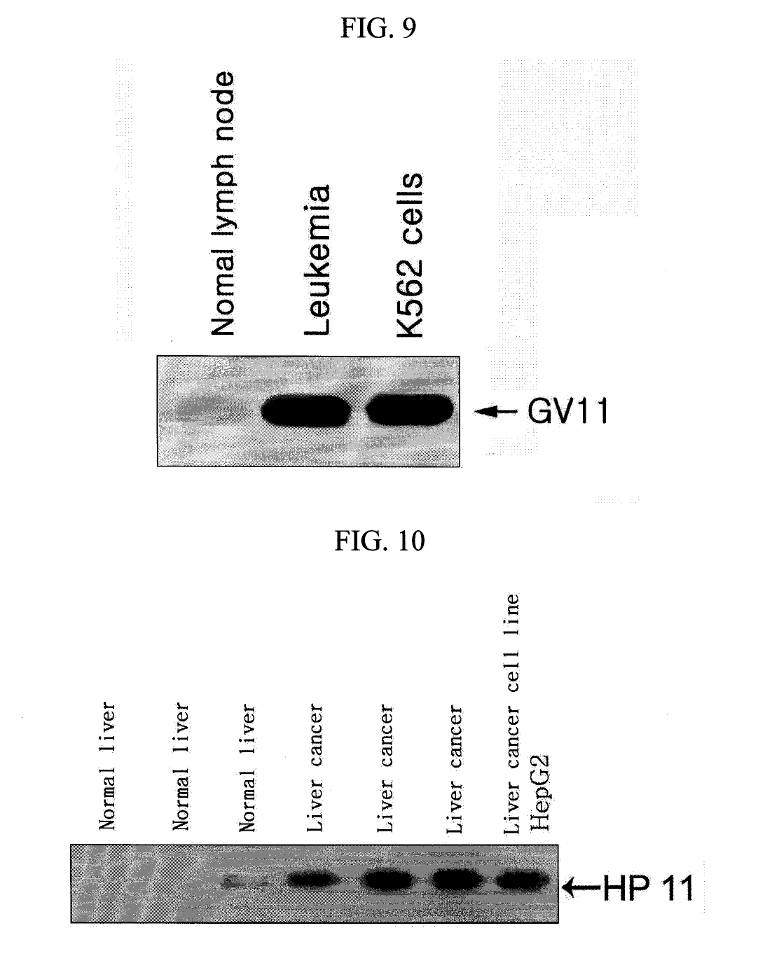

As shown in FIG. 9, it was revealed from the differential display (DD) that the gene was differentially expressed in the normal peripheral blood tissue, the leukemia tissue and the K-562 cell. As seen in FIG. 9, the 215-bp cDNA fragment GV11 was expressed in the leukemia tissue and the K-562 cancer cell, but very rarely expressed in the normal tissue.

The DNA sequence of the said gene corresponds to nucleotide sequence positions from 879 to 1,199 of SEQ ID NO: 37, which was designated “HP11” in the present invention.

The 321-bp cDNA fragment obtained above, namely HP11, was subject to the differential display reverse transcription-polymerase chain reaction (DDRT-PCR) using a 5′-random primer H-AP11 and a 3′-anchored primer H-T11G, and then confirmed using the electrophoresis. As shown in FIG. 10, it was revealed from the differential display (DD) that the gene was differentially expressed in the normal liver tissue, the liver cancer tissue and the HepG2 cell. As seen in FIG. 10, the 321-bp cDNA fragment HP11 was expressed in the liver cancer tissue and the HepG2 cancer cell, but not expressed or detected in the normal tissue.

The DNA sequence of the said gene corresponds to nucleotide sequence positions from 1,369 to 1,679 of SEQ ID NO: 41, which was designated “HP23” in the present invention.

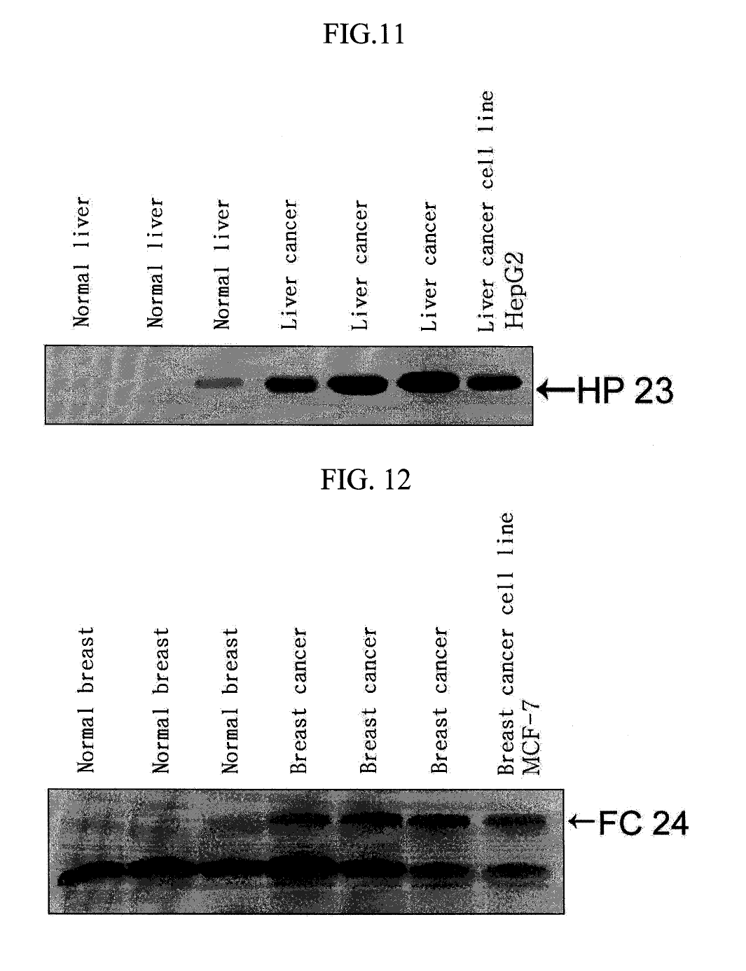

The 311-bp cDNA fragment obtained above, namely HP23, was subject to the differential display reverse transcription-polymerase chain reaction (DDRT-PCR) using a 5′-random primer H-AP23 and a 3′-anchored primer H-T11C, and then confirmed using the electrophoresis. As shown in FIG. 11, it was revealed from the differential display (DD) that the gene was differentially expressed in the normal liver tissue, the liver cancer tissue and the HepG2 cell. As seen in FIG. 11, the 311-bp cDNA fragment HP23 was expressed in the liver cancer tissue and the HepG2 cancer cell, but not expressed or rarely expressed in the normal tissue.

The DNA sequence of the said gene corresponds to nucleotide sequence positions from 992 to 1,247 of SEQ ID NO: 45, which was designated “FC24” in the present invention.

The 256-bp cDNA fragment obtained above, namely FC24, was subject to the differential display reverse transcription-polymerase chain reaction (DDRT-PCR) using a 5′-random primer H-AP24 and a 3′-anchored primer H-T11A, and then confirmed using the electrophoresis. As shown in FIG. 12, it was revealed from the differential display (DD) that the gene was differentially expressed in the normal breast tissue, the breast cancer tissue and the MCF-7 cell. As seen in FIG. 12, the 256-bp cDNA fragment FC24 was highly expressed in the breast cancer tissue and the MCF-7 cancer cell, but very rarely expressed in the normal tissue.

The DNA sequence of the said gene corresponds to nucleotide sequence positions from 587 to 778 of SEQ ID NO: 49, which was designated “FC54” in the present invention.

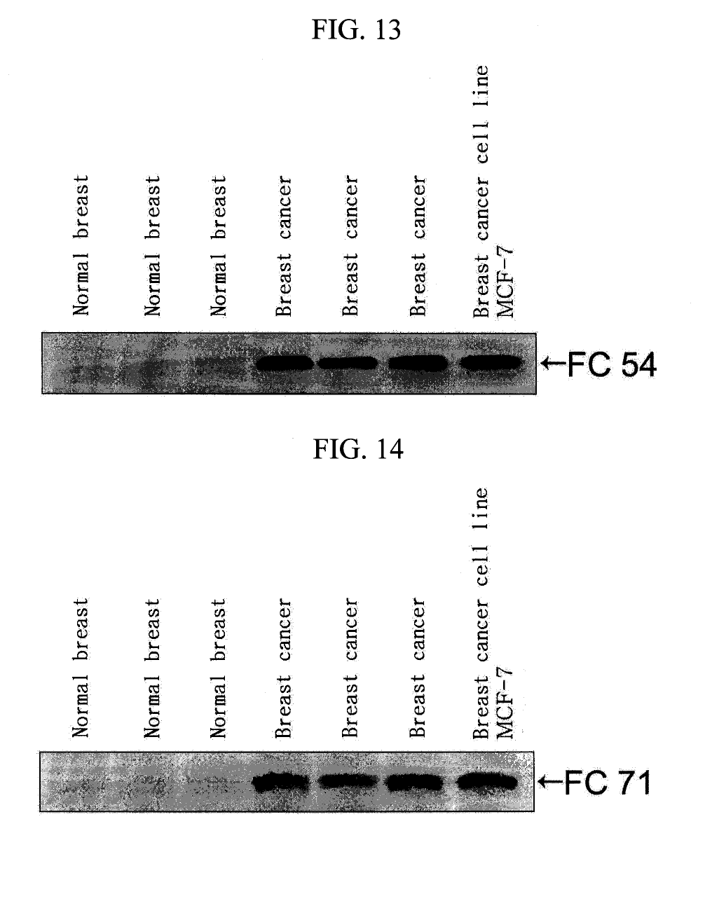

The 192-bp cDNA fragment obtained above, namely FC54, was subject to the differential display reverse transcription-polymerase chain reaction (DDRT-PCR) using a 5′-random primer H-AP5 and a 3′-anchored primer H-T11C, and then confirmed using the electrophoresis. As shown in FIG. 13, it was revealed from the differential display (DD) that the gene was differentially expressed in the normal breast tissue, the breast cancer tissue and the MCF-7 cell. As seen in FIG. 13, the 192-bp cDNA fragment FC54 was highly expressed in the breast cancer tissue and the MCF-7 cancer cell, but very rarely expressed in the normal tissue.

The DNA sequence of the said gene corresponds to nucleotide sequence positions from 1,348 to 1,619 of SEQ ID NO: 53, which was designated “FC71” in the present invention.

The 272-bp cDNA fragment obtained above, namely FC71, was subject to the differential display reverse transcription-polymerase chain reaction (DDRT-PCR) using a 5′-random primer H-AP7 and a 3′-anchored primer H-T11A, and then confirmed using the electrophoresis. As shown in FIG. 14, it was revealed from the differential display (DD) that the gene was differentially expressed in the normal breast tissue, the breast cancer tissue and the MCF-7 cell. As seen in FIG. 14, the 272-bp cDNA fragment FC71 was highly expressed in the breast cancer tissue and the MCF-7 cancer cell, but very rarely expressed in the normal tissue.

The DNA sequence of the said gene corresponds to nucleotide sequence positions from 418 to 599 of SEQ ID NO: 57, which was designated “BBCC5-5” in the present invention.

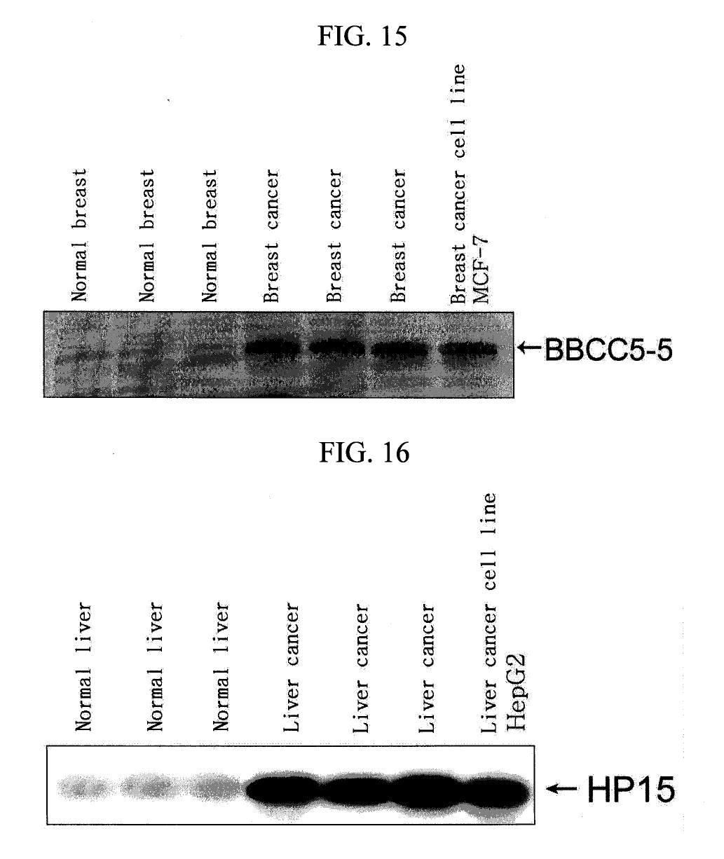

The 182-bp cDNA fragment obtained above, namely BBCC5-5, was subject to the differential display reverse transcription-polymerase chain reaction (DDRT-PCR) using a 5′-random primer H-AP5 and a 3′-anchored primer H-T11C, and then confirmed using the electrophoresis. As shown in FIG. 15, it was revealed from the differential display (DD) that the gene was differentially expressed in the normal breast tissue, the breast cancer tissue and the MCF-7 cell. As seen in FIG. 15, the 182-bp cDNA fragment BBCC5-5 was highly expressed in the breast cancer tissue and the MCF-7 cancer cell, but very rarely expressed in the normal tissue.

The DNA sequence of the said gene corresponds to nucleotide sequence positions from 1,533 to 1,877 of SEQ ID NO: 61, which was designated “HP15” in the present invention.

The 345-bp cDNA fragment obtained above, namely HP15, was subject to the differential display reverse transcription-polymerase chain reaction (DDRT-PCR) using a 5′-random primer H-AP15 and a 3′-anchored primer H-T11A, and then confirmed using the electrophoresis. As shown in FIG. 16, it was revealed from the differential display (DD) that the gene was differentially expressed in the normal liver tissue, the liver cancer tissue and the HepG2 cell. As seen in FIG. 16, the 345-bp cDNA fragment HP15 was expressed in the liver cancer tissue and the HepG2 cancer cell, but not expressed or rarely expressed in the normal tissue.

The DNA sequence of the said gene corresponds to nucleotide sequence positions from 292 to 477 of SEQ ID NO: 65, which was designated “FC4” in the present invention.

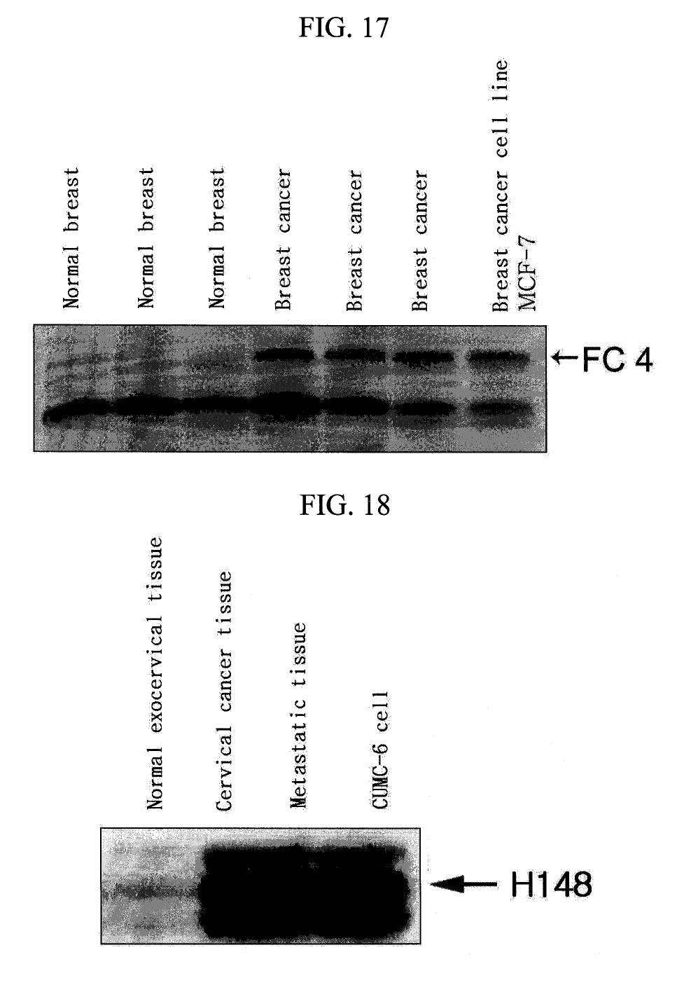

The 186-bp cDNA fragment obtained above, namely FC4, was subject to the differential display reverse transcription-polymerase chain reaction (DDRT-PCR) using a 5′-random primer H-AP4 and a 3′-anchored primer H-T11G, and then confirmed using the electrophoresis. As shown in FIG. 17, it was revealed from the differential display (DD) that the gene was differentially expressed in the normal breast tissue, the breast cancer tissue and the MCF-7 cell. As seen in FIG. 17, the 186-bp cDNA fragment FC4 was highly expressed in the breast cancer tissue and the MCF-7 cancer cell, but very rarely expressed in the normal tissue.

The DNA sequence of the said gene corresponds to nucleotide sequence positions from 769 to 989 of SEQ ID NO: 69, which was designated “H148” in the present invention.

The 221-bp cDNA fragment obtained above, namely H148, was subject to the differential display reverse transcription-polymerase chain reaction (DDRT-PCR) using a 5′-random primer H-AP14 and a 3′-anchored primer H-T11A, and then confirmed using the electrophoresis.

As shown in FIG. 18, it was revealed from the differential display (DD) that the gene was differentially expressed in the normal exocervical tissue, the metastatic lymph node tissue and the CUMC-6 cell. As seen in FIG. 18, the 221-bp cDNA fragment H148 was expressed in the cervical cancer tissue, the metastatic lymph node tissue and the CUMC-6 cancer cell, but very rarely expressed in the normal tissue.

The DNA sequence of the said gene corresponds to nucleotide sequence positions from 1,007 to 1,328 of SEQ ID NO: 73, which was designated “L738” in the present invention.

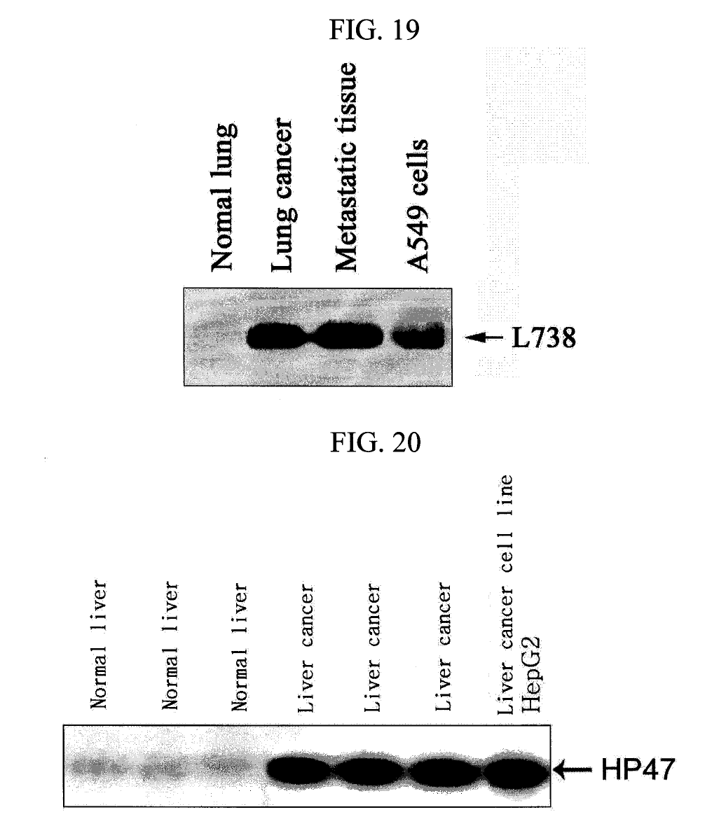

The 322-bp cDNA fragment obtained above, namely L738, was subject to the differential display reverse transcription-polymerase chain reaction (DDRT-PCR) using a 5′-random primer H-AP7 and a 3′-anchored primer H-T11G, and then confirmed using the electrophoresis. As shown in FIG. 19, it was revealed from the differential display (DD) that the gene was differentially expressed in the normal lung tissue, the left lung cancer tissue, the metastatic lung cancer tissue metastasized from the left lung to the right lung and the A549 lung cancer cell. As seen in FIG. 19, the 322-bp cDNA fragment L738 was expressed in the lung cancer tissue, the metastatic lung cancer and the A549 lung cancer cell, but not expressed or very rarely expressed in the normal lung tissue.

The DNA sequence of the said gene corresponds to nucleotide sequence positions from 879 to 1,199 of SEQ ID NO: 77, which was designated “HP47” in the present invention.

The 321-bp cDNA fragment obtained above, namely HP47, was subject to the differential display reverse transcription-polymerase chain reaction (DDRT-PCR) using a 5′-random primer H-AP4 and a 3′-anchored primer H-T11C, and then confirmed using the electrophoresis. As shown in FIG. 20, it was revealed from the differential display (DD) that the gene was differentially expressed in the normal liver tissue, the liver cancer tissue and the HepG2 cell. As seen in FIG. 20, the 321-bp cDNA fragment HP47 was expressed in the liver cancer tissue and the HepG2 cancer cell, but rarely expressed in the normal tissue.

The DNA sequence of the said gene corresponds to nucleotide sequence positions from 273 to 599 of SEQ ID NO: 81, which was designated “L690” in the present invention.

The 327-bp cDNA fragment obtained above, namely L690, was subject to the differential display reverse transcription-polymerase chain reaction (DDRT-PCR) using a 5′-random primer H-AP6 and a 3′-anchored primer H-T11A, and then confirmed using the electrophoresis.

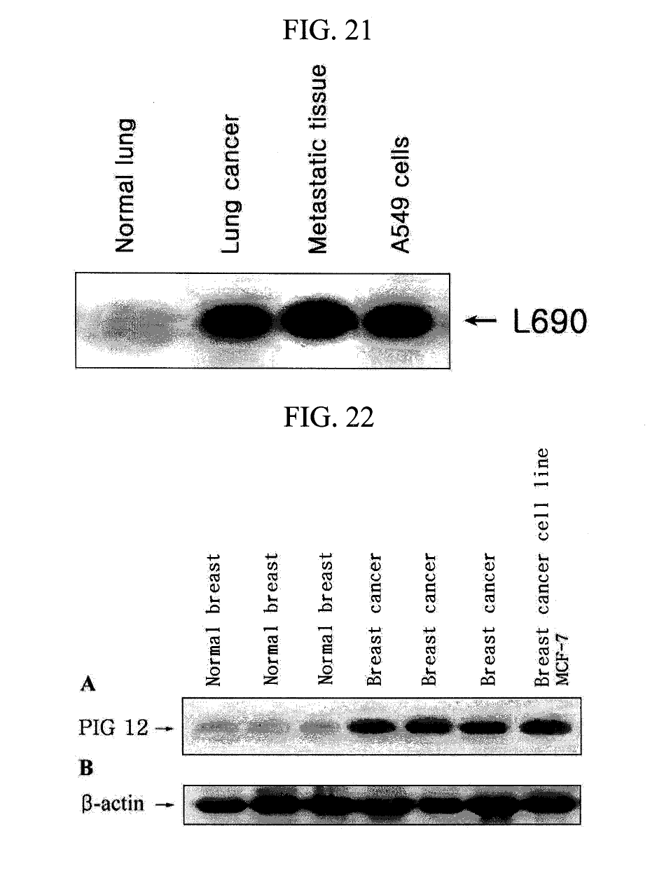

As shown in FIG. 21, it was revealed from the differential display (DD) that the gene was differentially expressed in the normal lung tissue, the left lung cancer tissue, the metastatic lung cancer tissue metastasized from the left lung to the right lung and the A549 lung cancer cell. As seen in FIG. 21, the 327-bp cDNA fragment L690 was expressed in the lung cancer tissue, the metastatic lung cancer tissue and the A549 lung cancer cell, but rarely expressed or not expressed in the normal lung tissue. Expecially, the 327-bp cDNA fragment L690 was the most expressed in the cancer tissue, for example the metastatic cancer tissue.

### Example 6

### cDNA Sequence Analysis of Full-Length Protooncogene

6-1. PIG12

The ³²P-labeled FC21 was used as the probe to screen a bacteriophage λgt11 human lung embryonic fibroblast cDNA library (Miki, T. et al., Gene 83: 137-146, 1989). A full-length PIG12 cDNA clone, in which the 1,258-bp fragment was inserted into the pCEV-LAC vector, was obtained from the human lung embryonic fibroblast cDNA library, and then deposited with Accession No. AY550973 in the U.S. GenBank database on Feb. 16, 2004 (Scheduled Release Date: Dec. 31, 2005).

A DNA sequence of the AY550973 gene was similar to that of the Homo sapiens gene finger protein 193 (ZNF193) gene deposited with Accession No. NM_{—}006299 in the database. Contrary to its functions as reported previously, it was however found from this study results that the AY550973 gene is closely relevant to various tumorigeneses, especially including the breast cancer. As the study result, it was found that a PIG12 protooncogene is rarely expressed in various normal human tissues including the breast tissue, while its expression is significantly increased in various cancer tissues including the breast cancer.

The full-length DNA sequence of the PIG12 consisting of 1,258 bp was set forth in SEQ ID NO: 1.

In the DNA sequence of SEQ ID NO: 1, it is estimated that a full-length open reading frame of the protooncogene of the present invention corresponds to nucleotide sequence positions from 68 to 1,252, and encodes a protein consisting of 394 amino acids of SEQ ID NO: 2.

6-2. PIG18

The ³²P-labeled MC113 was used as the probe to screen a bacteriophage λgt11 human lung embryonic fibroblast cDNA library (Miki, T. et al., Gene 83: 137-146, 1989). A full-length PIG18 cDNA clone, in which the 2,403-bp fragment was inserted into the pCEV-LAC vector, was obtained from the human lung embryonic fibroblast cDNA library, and then deposited with Accession No. AY771596 in the U.S. GenBank database on Oct. 5, 2004 (Scheduled Release Date: Dec. 31, 2005).

The PIG18 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the PIG18 gene was ligated by T4 DNA ligase to prepare PIG18 plasmid DNA, and then E. coli DH5 α was transformed with the ligated clone.

The full-length DNA sequence of the PIG18 consisting of 2,403 bp was set forth in SEQ ID NO: 5.

In the DNA sequence of SEQ ID NO: 5, it is estimated that a full-length open reading frame of the protooncogene of the present invention corresponds to nucleotide sequence positions from 875 to 1,063, and encodes a protein consisting of 62 amino acids of SEQ ID NO: 6.

6-3. PIG23

The ³²P-labeled CA338d was used as the probe to screen a bacteriophage A gt11 human lung embryonic fibroblast cDNA library (Miki, T. et al., Gene 83: 137-146, 1989). A full-length PIG23 cDNA clone, in which the 2,150-bp fragment was inserted into the pCEV-LAC vector, was obtained from the human lung embryonic fibroblast cDNA library, and then deposited with Accession No. AY826819 in the U.S. GenBank database on Oct. 23, 2004 (Scheduled Release Date: Dec. 31, 2005).

The PIG23 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the PIG23 gene was ligated by T4 DNA ligase to prepare PIG23 plasmid DNA, and then E. coli DH5 α was transformed with the ligated clone.

The full-length DNA sequence of the PIG23 consisting of 2,150 bp was set forth in SEQ ID NO: 9.

In the DNA sequence of SEQ ID NO: 9, it is estimated that a full-length open reading frame of the protooncogene of the present invention corresponds to nucleotide sequence positions from 25 to 1,953, and encodes a protein consisting of 642 amino acids of SEQ ID NO: 10.

6-4. PIG27

The ³²P-labeled H124 was used as the probe to screen a bacteriophage λgt11 human lung embryonic fibroblast cDNA library (Miki, T. et al., Gene 83: 137-146, 1989). A full-length PIG27 cDNA clone, in which the 446-bp fragment was inserted into the pCEV-LAC vector, was obtained from the human lung embryonic fibroblast cDNA library, and then deposited with Accession No. AY453399 in the U.S. GenBank database on Oct. 30, 2003 (Scheduled Release Date: Mar. 31, 2005).

The PIG27 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the PIG27 gene was ligated by T4 DNA ligase to prepare PIG27 plasmid DNA, and then E. coli DH5 α was transformed with the ligated clone.

The full-length DNA sequence of the PIG27 consisting of 446 bp was set forth in SEQ ID NO: 13.

In the DNA sequence of SEQ ID NO: 13, it is estimated that a full-length open reading frame of the protooncogene of the present invention corresponds to nucleotide sequence positions from 20 to 337, and encodes a protein consisting of 105 amino acids of SEQ ID NO: 14.

6-5. PIG28

The ³²P-labeled H1122 was used as the probe to screen a bacteriophage λgt11 human lung embryonic fibroblast cDNA library (Miki, T. et al., Gene 83: 137-146, 1989). A full-length PIG28 cDNA clone, in which the 1,024-bp fragment was inserted into the pCEV-LAC vector, was obtained from the human lung embryonic fibroblast cDNA library, and then deposited with Accession No. AY453398 in the U.S. GenBank database on Oct. 30, 2003 (Scheduled Release Date: Mar. 31, 2005).

The PIG28 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the PIG28 gene was ligated by T4 DNA ligase to prepare PIG28 plasmid DNA, and then E. coli DH5 α was transformed with the ligated clone.

The full-length DNA sequence of the PIG28 consisting of 1,024 bp was set forth in SEQ ID NO: 17.

In the DNA sequence of SEQ ID NO: 17, it is estimated that a full-length open reading frame of the protooncogene of the present invention corresponds to nucleotide sequence positions from 33 to 998, and encodes a protein consisting of 321 amino acids of SEQ ID NO: 18.

6-6. PIG30

The ³²P-labeled FC23 was used as the probe to screen a bacteriophage λgt11 human lung embryonic fibroblast cDNA library (Miki, T. et al., Gene 83: 137-146, 1989). A full-length PIG30 cDNA clone, in which the 2,152-bp fragment was inserted into the pCEV-LAC vector, was obtained from the human lung embryonic fibroblast cDNA library, and then deposited with Accession No. AY550975 in the U.S. GenBank database on Feb. 16, 2004 (Scheduled Release Date: Dec. 31, 2005).

A DNA sequence of the AY550975 gene was similar to that of the Homo sapiens calpain 1, (mu/I) large subunit (CAPN1) gene deposited with Accession No. NM_{—}005186 in the database. Contrary to its functions as reported previously, it was however found from this study results that the AY550975 gene is closely relevant to various tumorigeneses, especially including the breast cancer. As the study result, it was found that a PIG30 protooncogene is rarely expressed in various normal human tissues including the breast tissue, while its expression is significantly increased in various cancer tissues including the breast cancer.

The full-length DNA sequence of the PIG30 consisting of 2,152 bp was set forth in SEQ ID NO: 21.

In the DNA sequence of SEQ ID NO: 21, it is estimated that a full-length open reading frame of the protooncogene of the present invention corresponds to nucleotide sequence positions from 6 to 2,150, and encodes a protein consisting of 714 amino acids of SEQ ID NO: 22.

6-7. PIG31

The ³²P-labeled FC34 was used as the probe to screen a bacteriophage λgt11 human lung embryonic fibroblast cDNA library (Miki, T. et al., Gene 83: 137-146, 1989). A full-length PIG31 cDNA clone, in which the 2,246-bp fragment was inserted into the pCEV-LAC vector, was obtained from the human lung embryonic fibroblast cDNA library, and then deposited with Accession No. AY644768 in the U.S. GenBank database on Jun. 3, 2004 (Scheduled Release Date: Dec. 31, 2005). A DNA sequence of the AY644768 gene was similar to that of the Homo sapiens golgin-84 mRNA gene deposited with Accession No. AF085199 in the database. Contrary to its functions as reported previously, it was however found from this study results that the AY644768 gene is closely relevant to various tumorigeneses, especially including the breast cancer. As the study result, it was found that a PIG31 protooncogene is rarely expressed in various normal human tissues including the breast tissue, while its expression is significantly increased in various cancer tissues including the breast cancer.

The full-length DNA sequence of the PIG31 consisting of 2,246 bp was set forth in SEQ ID NO: 25.

In the DNA sequence of SEQ ID NO: 25, it is estimated that a full-length open reading frame of the protooncogene of the present invention corresponds to nucleotide sequence positions from 37 to 2,232, and encodes a protein consisting of 731 amino acids of SEQ ID NO: 26.

6-8. PIG38

The ³²P-labeled HP103 was used as the probe to screen a bacteriophage λgt11 human lung embryonic fibroblast cDNA library (Miki, T. et al., Gene 83: 137-146, 1989). A full-length PIG38 cDNA clone, in which the 1,973-bp fragment was inserted into the pCEV-LAC vector, was obtained from the human lung embryonic fibroblast cDNA library, and then deposited with Accession No. AY513282 in the U.S. GenBank database on Dec. 24, 2003 (Scheduled Release Date: Dec. 31, 2005). A DNA sequence of the AY513282 gene was similar to that of the Homo sapiens hypothetical protein FLJ10094 gene deposited with Accession No. BC024178 in the database. However, functions of the gene remain to be known. Contrary to its functions as reported previously, it was however found from this study results that the AY513282 gene is closely relevant to various tumorigeneses, especially including the liver cancer. As the study result, it was found that a PIG38 protooncogene is rarely expressed in various normal human tissues including the liver tissue, while its expression is significantly increased in various cancer tissues including the liver cancer.

The full-length DNA sequence of the PIG38 consisting of 1,973 bp was set forth in SEQ ID NO: 29.

In the DNA sequence of SEQ ID NO: 29, it is estimated that a full-length open reading frame of the protooncogene of the present invention corresponds to nucleotide sequence positions from 25 to 1,956, and encodes a protein consisting of 643 amino acids of SEQ ID NO: 30.

6-9. PIG40

The ³²P-labeled GV11 was used as the probe to screen a bacteriophage λgt11 human lung embryonic fibroblast cDNA library (Miki, T. et al., Gene 83: 137-146, 1989). A full-length PIG40 cDNA clone, in which the 1,586-bp fragment was inserted into the pCEV-LAC vector, was obtained from the human lung embryonic fibroblast cDNA library, and then deposited with Accession No. AY762100 in the U.S. GenBank database on Sep. 23, 2004 (Scheduled Release Date: Mar. 31, 2005).

The PIG40 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the PIG40 gene was ligated by T4 DNA ligase to prepare PIG40 plasmid DNA, and then E. coli DH5 α was transformed with the ligated clone.

The full-length DNA sequence of the PIG40 consisting of 1,586 bp was set forth in SEQ ID NO: 33.

In the DNA sequence of SEQ ID NO: 33, it is estimated that a full-length open reading frame of the protooncogene of the present invention corresponds to nucleotide sequence positions from 36 to 1,541, and encodes a protein consisting of 501 amino acids of SEQ ID NO: 34.

6-10. PIG43

The ³²P-labeled HP11 was used as the probe to screen a bacteriophage A gt11 human lung embryonic fibroblast cDNA library (Miki, T. et al., Gene 83: 137-146, 1989). A full-length PIG43 cDNA clone, in which the 1,245-bp fragment was inserted into the pCEV-LAC vector, was obtained from the human lung embryonic fibroblast cDNA library, and then deposited with Accession No. AY513283 in the U.S. GenBank database on Dec. 25, 2003 (Scheduled Release Date: Dec. 31, 2005). A DNA sequence of the AY513283 gene was similar to that of the Homo sapiens glutamate-ammonia ligase (glutamine synthase) (GLUL) gene deposited with Accession No. NM_{—}002065 in the database, but their expressed proteins are different to each other. Contrary to its functions as reported previously, it was however found from this study results that the AY513283 gene is closely relevant to various tumorigeneses, especially including the liver cancer. As the study result, it was found that a PIG43 protooncogene is rarely expressed in various normal human tissues including the liver tissue, while its expression is significantly increased in various cancer tissues including the liver cancer.

The full-length DNA sequence of the PIG43 consisting of 1,245 bp was set forth in SEQ ID NO: 37.

In the DNA sequence of SEQ ID NO: 37, it is estimated that a full-length open reading frame of the protooncogene of the present invention corresponds to nucleotide sequence positions from 57 to 758, and encodes a protein consisting of 233 amino acids of SEQ ID NO: 38.

6-11. PIG44

The ³²P-labeled HP23 was used as the probe to screen a bacteriophage λgt11 human lung embryonic fibroblast cDNA library (Miki, T. et al., Gene 83: 137-146, 1989). A full-length PIG44 cDNA clone, in which the 1,721-bp fragment was inserted into the pCEV-LAC vector, was obtained from the human lung embryonic fibroblast cDNA library, and then deposited with Accession No. AY513284 in the U.S. GenBank database on Dec. 25, 2003 (Scheduled Release Date: Dec. 31, 2005). A DNA sequence of the AY513282 gene was similar to that of the Homo sapiens hypothetical protein FLJ 0094 gene deposited with Accession No. AB037773 in the database, but their expressed proteins are different to each other. Contrary to its functions as reported previously, it was however found from this study results that the AY513284 gene is closely relevant to various tumorigeneses, especially including the liver cancer. As the study result, it was found that a PIG44 protooncogene is rarely expressed in various normal human tissues including the liver tissue, while its expression is significantly increased in various cancer tissues including the liver cancer.

The full-length DNA sequence of the PIG44 consisting of 1,721 bp was set forth in SEQ ID NO: 41.

In the DNA sequence of SEQ ID NO: 41, it is estimated that a full-length open reading frame of the protooncogene of the present invention corresponds to nucleotide sequence positions from 55 to 1,512, and encodes a protein consisting of 485 amino acids of SEQ ID NO: 42.

6-12. PIG46

The ³²P-labeled FC24 was used as the probe to screen a bacteriophage λgt11 human lung embryonic fibroblast cDNA library (Miki, T. et al., Gene 83: 137-146, 1989). A full-length PIG46 cDNA clone, in which the 1,312-bp fragment was inserted into the pCEV-LAC vector, was obtained from the human lung embryonic fibroblast cDNA library, and then deposited with Accession No. AY762101 in the U.S. GenBank database on Sep. 23, 2004 (Scheduled Release Date: Dec. 31, 2005). A DNA sequence of the AY762101 gene was similar to that of the Homo sapiens keratin 18 (KRT18), transcriptional variant 1 gene deposited with Accession No. NM_{—}000224 in the database. Contrary to its functions as reported previously, it was however found from this study results that the AY762101 gene is closely relevant to various tumorigeneses, especially including the breast cancer. As the study result, it was found that a PIG46 protooncogene is rarely expressed or not expressed in various normal human tissues including the breast tissue, while its expression is significantly increased in various cancer tissues including the breast cancer.

The full-length DNA sequence of the PIG46 consisting of 1,312 bp was set forth in SEQ ID NO:45.

In the DNA sequence of SEQ ID NO: 45, it is estimated that a full-length open reading frame of the protooncogene of the present invention corresponds to nucleotide sequence positions from 5 to 1,297, and encodes a protein consisting of 430 amino acids of SEQ ID NO: 46.

6-13. PIG47

The ³²P-labeled FC54 was used as the probe to screen a bacteriophage λgt11 human lung embryonic fibroblast cDNA library (Miki, T. et al., Gene 83: 137-146, 1989). A full-length PIG47 cDNA clone, in which the 827-bp fragment was inserted into the pCEV-LAC vector, was obtained from the human lung embryonic fibroblast cDNA library, and then deposited with Accession No. AY871272 in the U.S. GenBank database on Jan. 1, 2005 (Scheduled Release Date: Oct. 1, 2006). A DNA sequence of the AY871272 gene was similar to that of the Homo sapiens ATP sythetase, H+ transporting, mitochondria F0 complex, subunit b, isoform 1 (ATP5F1) gene deposited with Accession No. NM_{—}001688 in the database. Contrary to its functions as reported previously, it was however found from this study results that the AY871272 gene is closely relevant to various tumorigeneses, especially including the breast cancer. As the study result, it was found that a PIG47 protooncogene is rarely expressed in various normal human tissues including the breast tissue, while its expression is significantly increased in various cancer tissues including the breast cancer. The full-length DNA sequence of the PIG47 consisting of 827 bp was set forth in SEQ ID NO: 49.

In the DNA sequence of SEQ ID NO: 49, it is estimated that a full-length open reading frame of the protooncogene of the present invention corresponds to nucleotide sequence positions from 56 to 826, and encodes a protein consisting of 256 amino acids of SEQ ID NO: 50.

6-14. PIG48

The ³²P-labeled FC71 was used as the probe to screen a bacteriophage λgt11 human lung embryonic fibroblast cDNA library (Miki, T. et al., Gene 83: 137-146, 1989). A full-length PIG48 cDNA clone, in which the 1,707-bp fragment was inserted into the pCEV-LAC vector, was obtained from the human lung embryonic fibroblast cDNA library, and then deposited with Accession No. AY524046 in the U.S. GenBank database on Jan. 12, 2004 (Scheduled Release Date: Dec. 31, 2005). A DNA sequence of the AY524046 gene was similar to that of the Homo sapiens TCP1-containing chaperonin, subunit 3 (gamma) (CCT3) gene deposited with Accession No. NM_{—}005998 in the database. Contrary to its functions as reported previously, it was however found from this study results that the AY524046 gene is closely relevant to various tumorigeneses, especially including the breast cancer. As the study result, it was found that a PIG48 protooncogene is rarely expressed in various normal human tissues including the breast tissue, while its expression is significantly increased in various cancer tissues including the breast cancer.

The full-length DNA sequence of the PIG48 consisting of 1,707 bp was set forth in SEQ ID NO: 53.

In the DNA sequence of SEQ ID NO: 53, it is estimated that a full-length open reading frame of the protooncogene of the present invention corresponds to nucleotide sequence positions from 57 to 1,694, and encodes a protein consisting of 545 amino acids of SEQ ID NO: 54.

6-15. PIG50

The ³²P-labeled BBCC5-5 was used as the probe to screen a bacteriophage λgt11 human lung embryonic fibroblast cDNA library (Miki, T. et al., Gene 83: 137-146, 1989). A full-length PIG50 cDNA clone, in which the 643-bp fragment was inserted into the pCEV-LAC vector, was obtained from the human lung embryonic fibroblast cDNA library, and then deposited with Accession No. AY542309 in the U.S. GenBank database on Feb. 5, 2004 (Scheduled Release Date: Dec. 31, 2005). A DNA sequence of the AY542309 gene was similar to that of the Homo sapiens cDNA FLJ20497 fis gene deposited with Accession No. AK000504 in the database. Contrary to its functions as reported previously, it was however found from this study results that the AY542309 gene is closely relevant to various tumorigeneses, especially including the breast cancer. As the study result, it was found that a PIG50 protooncogene is rarely expressed in various normal human tissues including the breast tissue, while its expression is significantly increased in various cancer tissues including the breast cancer.

The full-length DNA sequence of the PIG50 consisting of 643 bp was set forth in SEQ ID NO: 57.

In the DNA sequence of SEQ ID NO: 57, it is estimated that a full-length open reading frame of the protooncogene of the present invention corresponds to nucleotide sequence positions from 2 to 595, and encodes a protein consisting of 197 amino acids of SEQ ID NO: 58.

6-16. PIG54

The ³²P-labeled HP15 was used as the probe to screen a bacteriophage λgt11 human lung embryonic fibroblast cDNA library (Miki, T. et al., Gene 83: 137-146, 1989). A full-length PIG44 cDNA clone, in which the 1,936-bp fragment was inserted into the pCEV-LAC vector, was obtained from the human lung embryonic fibroblast cDNA library, and then deposited with Accession No. AY550968 in the U.S. GenBank database on Feb. 16, 2004 (Scheduled Release Date: Dec. 31, 2005). A DNA sequence of the AY550968 gene was similar to that of the Homo sapiens SCC-112 protein gene deposited with Accession No. BC041361 in the database. Contrary to its functions as reported previously, it was however found from this study results that the AY550968 gene is closely relevant to various tumorigeneses, especially including the liver cancer. As the study result, it was found that a PIG54 protooncogene is rarely expressed in various normal human tissues including the liver tissue, while its expression is significantly increased in various cancer tissues including the liver cancer.

The full-length DNA sequence of the PIG54 consisting of 1,936 bp was set forth in SEQ ID NO: 61.

In the DNA sequence of SEQ ID NO: 61, it is estimated that a full-length open reading frame of the protooncogene of the present invention corresponds to nucleotide sequence positions from 38 to 1,840, and encodes a protein consisting of 600 amino acids of SEQ ID NO: 62.

6-17. PIG55

The ³²P-labeled FC4 was used as the probe to screen a bacteriophage λgt11 human lung embryonic fibroblast cDNA library (Miki, T. et al., Gene 83: 137-146, 1989). A full-length PIG55 cDNA clone, in which the 526-bp fragment was inserted into the pCEV-LAC vector, was obtained from the human lung embryonic fibroblast cDNA library, and then deposited with Accession No. AY644767 in the U.S. GenBank database on Jun. 2, 2004 (Scheduled Release Date: Dec. 31, 2005). A DNA sequence of the AY644767 gene was similar to that of the Homo sapiens nuclear cap binding protein subunit 2, 20 kDa (NCBP2) gene deposited with Accession No. NM_{—}007362 in the database. Contrary to its functions as reported previously, it was however found from this study results that the AY644767 gene is closely relevant to various tumorigeneses, especially including the breast cancer. As the study result, it was found that a PIG55 protooncogene is rarely expressed in various normal human tissues including the breast tissue, while its expression is significantly increased in various cancer tissues including the breast cancer.

The full-length DNA sequence of the PIG55 consisting of 526 bp was set forth in SEQ ID NO: 65.

In the DNA sequence of SEQ ID NO: 65, it is estimated that a full-length open reading frame of the protooncogene of the present invention corresponds to nucleotide sequence positions from 15 to 485, and encodes a protein consisting of 156 amino acids of SEQ ID NO: 66.

6-18. GIG9

The ³²P-labeled H148 was used as the probe to screen a bacteriophage λgt11 human lung embryonic fibroblast cDNA library (Miki, T. et al., Gene 83: 137-146, 1989). A full-length GIG9 cDNA clone, in which the 1,008-bp fragment was inserted into the pCEV-LAC vector, was obtained from the human lung embryonic fibroblast cDNA library, and then deposited with Accession No. AY453396 in the U.S. GenBank database on Oct. 29, 2003 (Scheduled Release Date: Dec. 31, 2005).

The GIG9 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the GIG9 gene was ligated by T4 DNA ligase to prepare GIG9 plasmid DNA, and then E. coli DH5 α was transformed with the ligated clone.

The full-length DNA sequence of the GIG9 consisting of 1,008 bp was set forth in SEQ ID NO: 69.

In the DNA sequence of SEQ ID NO: 69, it is estimated that a full-length open reading frame of the protooncogene of the present invention corresponds to nucleotide sequence positions from 1 to 1,008, and encodes a protein consisting of 335 amino acids of SEQ ID NO: 70.

6-19. HLC-9

A bacteriophage λgt11 human lung embryonic fibroblast cDNA library (Miki, T. et al., Gene 83: 137-146, 1989) was screened using the ³²P-labeled L738 as the probe, and therefore a full-length gene containing the L738 cDNA sequence was obtained. Two full-length genes were obtained from the human lung embryonic fibroblast cDNA library; one of the obtained genes is a full-length HLC9 cDNA clone in which the 1,382-bp fragment was inserted into the pCEV-LAC vector, and then deposited with Accession No. AY189686 in the U.S. GenBank database on Nov. 30, 2002 (Scheduled Release Date: May 1, 2004).

The HLC9 clone inserted into the λpCEV vector was cleaved by the restriction enzyme NotI and isolated from the phage in a form of ampicillin-resistant pCEV-LAC phagemid vector (See the above reference).

The pCEV-LAC vector containing the HLC9 gene was ligated by T4 DNA ligase to prepare HLC9 plasmid DNA, and then E. coli DH5 α was transformed with the ligated clone.

The full-length DNA sequence of the HLC9 consisting of 1,382 bp was set forth in SEQ ID NO: 73.

In the DNA sequence of SEQ ID NO: 73, it is estimated that a full-length open reading frame of the protooncogene of the present invention corresponds to nucleotide sequence positions from 27 to 1,370, and encodes a protein consisting of 447 amino acids of SEQ ID NO: 74.

6-20. GIG18

The ³²P-labeled HP47 was used as the probe to screen a bacteriophage λgt11 human lung embryonic fibroblast cDNA library (Miki, T. et al., Gene 83: 137-146, 1989). A full-length GIG18 cDNA clone, in which the 1,301-bp fragment was inserted into the pCEV-LAC vector, was obtained from the human lung embryonic fibroblast cDNA library, and then deposited with Accession No. AY513279 in the U.S. GenBank database on Dec. 24, 2003 (Scheduled Release Date: Dec. 31, 2005). It was confirmed that a DNA sequence of the AY513279 gene was similar to that of the Homo sapiens glutamic-oxaloacetic transaminase 1, soluble (aspartate aminotransferase 1) (GOT1), mRNA gene deposited with Accession No. NM_{—}002079 in the database. Contrary to its functions as reported previously, it was however found from this study results that the AY513279 gene is closely relevant to various tumorigeneses, especially including the liver cancer. As the study result, it was found that an GIG18 protooncogene is rarely expressed in various normal human tissues including the liver tissue, while its expression is significantly increased in various cancer tissues including the liver cancer. The full-length DNA sequence of the GIG18 consisting of 1,301 bp was set forth in SEQ ID NO: 77.

In the DNA sequence of SEQ ID NO: 77, it is estimated that a full-length open reading frame of the protooncogene of the present invention corresponds to nucleotide sequence positions from 3 to 1,244, and encodes a protein consisting of 413 amino acids of SEQ ID NO: 78.

6-21. MIG22

The ³²P-labeled L690 was used as the probe to screen a bacteriophage λgt11 human lung embryonic fibroblast cDNA library (Miki, T. et al., Gene 83: 137-146, 1989). A full-length MIG22 cDNA clone, in which the 749-bp fragment was inserted into the pCEV-LAC vector, was obtained from the human lung embryonic fibroblast cDNA library, and then deposited with Accession No. AY771595 in the U.S. GenBank database on Oct. 5, 2004 (Scheduled Release Date: Dec. 31, 2005).

The MIG22 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the MIG22 gene was ligated by T4 DNA ligase to prepare MIG22 plasmid DNA, and then E. coli DH5 α was transformed with the ligated clone.

In the DNA sequence of SEQ ID NO: 81, it is estimated that a full-length open reading frame of the protooncogene of the present invention corresponds to nucleotide sequence positions from 15 to 734, and encodes a protein consisting of 239 amino acids of SEQ ID NO: 82.

### Example 7

### Northern Blotting Analysis of Protooncogenes in Various Cells

7-1. PIG12, PIG30, PIG31, PIG46, PIG47, PIG48, PIG50 and PIG55

The total RNA samples were extracted from the normal breast tissue, the breast cancer tissue and the breast cancer cell line MCF-7 in the same manner as in Example 1-1.

In order to determine an expression level of each of the PIG genes, 20 μg of each of the total denatured RNA samples extracted from each of the tissues and the cell line was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed FC21 cDNA probe prepared using the Rediprime II random prime labelling system ((Amersham, United Kingdom). The northern blotting analysis was repeated twice, and therefore the resultant blots were quantitified with the densitometer and normalized with the β-actin.

FIG. 22 shows a northern blotting result to determine whether or not the PIG12 protooncogene is expressed in the normal breast tissue, the breast cancer tissue and the breast cancer cell line (MCF-7). As shown in FIG. 22, it was revealed that the expression level of the MIG3 protooncogene was significantly increased in the breast cancer tissue and the breast cancer cell line MCF-7, but very low or not detected in the normal tissue. In FIG. 22, a lane “Normal” represents the normal breast tissue, a lane “Cancer” represents the breast cancer tissue, and a lane “MCF-7” represents the breast cancer cell line. A bottom of FIG. 22 shows the northern blotting result indicating whether or not β-actin mRNA is transcribed by hybridizing the same sample with β-actin probe.

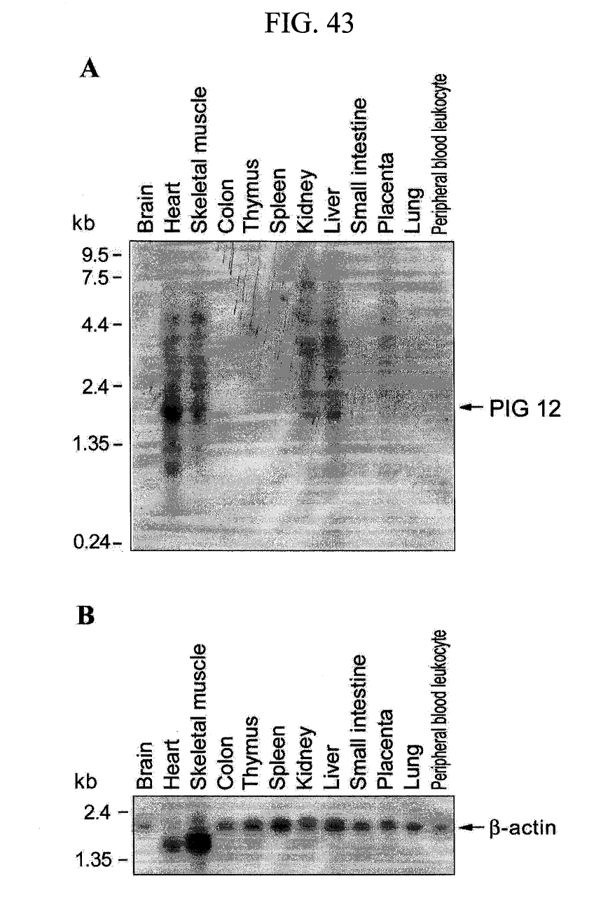

FIG. 43 shows a northern blotting result to determine whether or not the PIG12 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 43 shows the northern blotting result indicating whether or not β-actin mRNA is transcribed by hybridizing the same sample with β-actin probe. As shown in FIG. 43, it was revealed that the PIG12 mRNA transcript (approximately 2.0 kb) was not expressed in the various normal tissues.

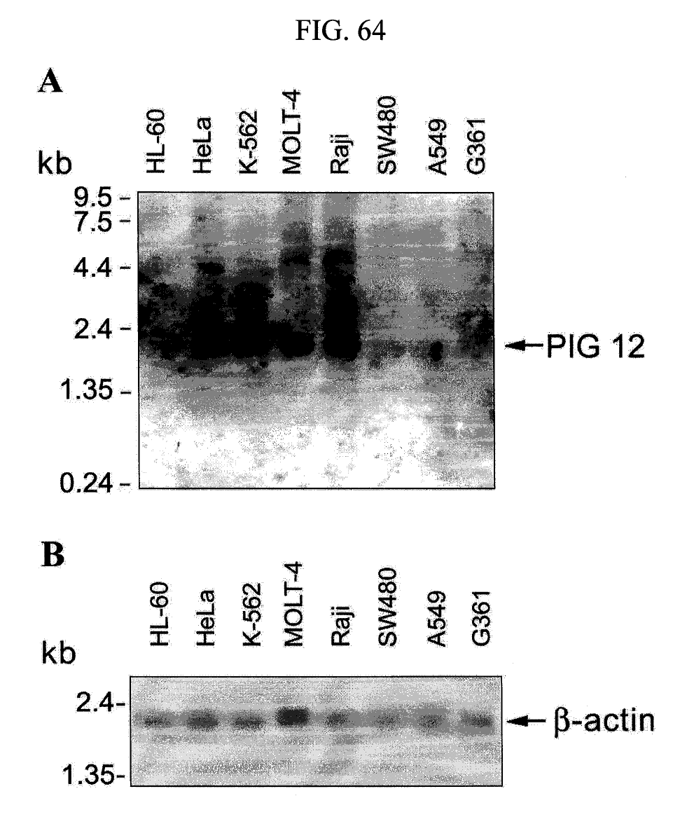

FIG. 64 shows a northern blotting result to determine whether or not the PIG12 protooncogene is expressed in the human cancer cell lines, for example HL-60, HeLa, K-562, MOLT-4, Raji, SW480, A549 and G361 (Clontech). A bottom of FIG. 64 shows the northern blotting result indicating whether or not β-actin mRNA is transcribed by hybridizing the same sample with β-actin probe. As shown in FIG. 64, it was revealed that the PIG12 mRNA transcript was very highly expressed in the HeLa uterine cancer cell line, the chronic myelogenous leukemia cell line K-562, the lymphoblastic leukaemia cell line MOLT-4 and the Burkitt lymphoma cell line Raji, but not expressed in the promyelocyte leukemia cell line HL-60, the colon cancer cell line SW480, the skin cancer cell line G361 and the lung cancer cell line A549.

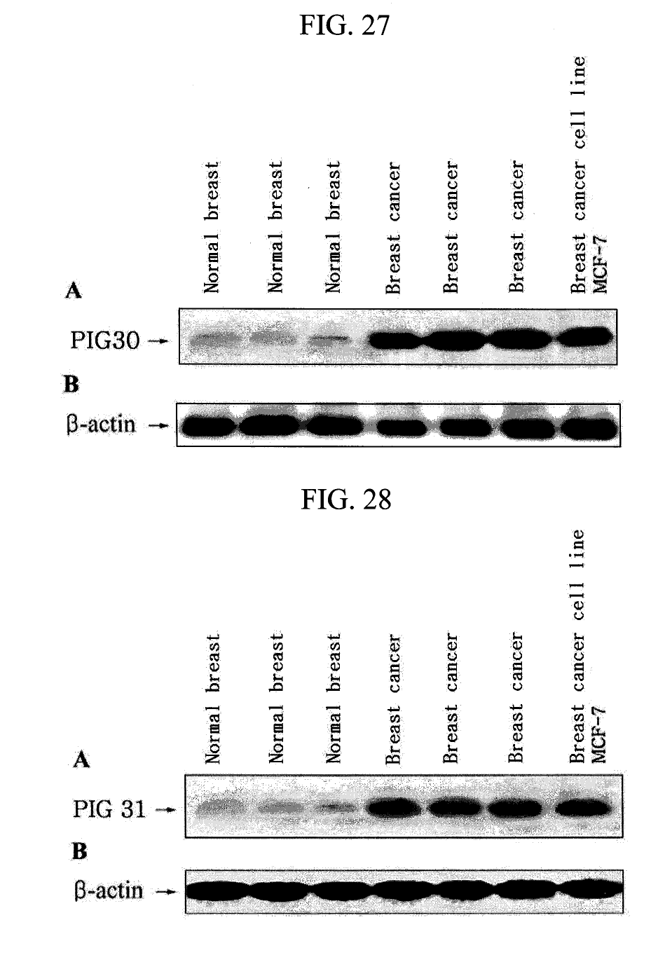

FIG. 27 shows a northern blotting result to determine whether or not the PIG30 protooncogene is expressed in the normal breast tissue, the breast cancer tissue and the breast cancer cell line (MCF-7). As shown in FIG. 27, it was revealed that the expression level of the PIG30 protooncogene was significantly increased in the breast cancer tissue and the breast cancer cell line MCF-7, but very low or not detected in the normal tissue. In FIG. 27, a lane “Normal” represents the normal breast tissue, a lane “Cancer” represents the breast cancer tissue, and a lane “MCF-7” represents the breast cancer cell line. A bottom of FIG. 27 shows the northern blotting result indicating whether or not β-actin mRNA is transcribed by hybridizing the same sample with β-actin probe.

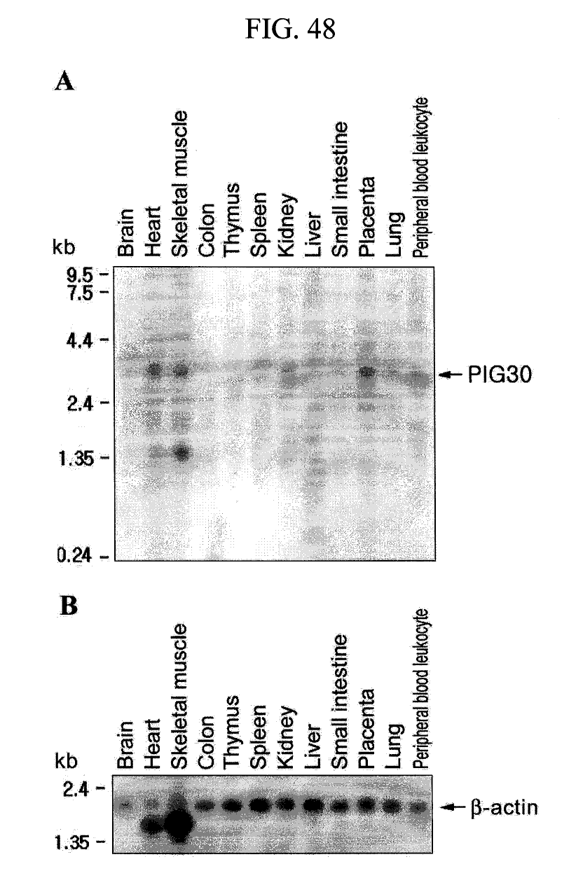

FIG. 48 shows a northern blotting result to determine whether or not the PIG30 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 48 shows the northern blotting result indicating whether or not β-actin mRNA is transcribed by hybridizing the same sample with β-actin probe. As shown in FIG. 48, it was revealed that the PIG30 mRNA transcript (approximately 3.5 kb) was very rarely expressed in the various normal tissues.

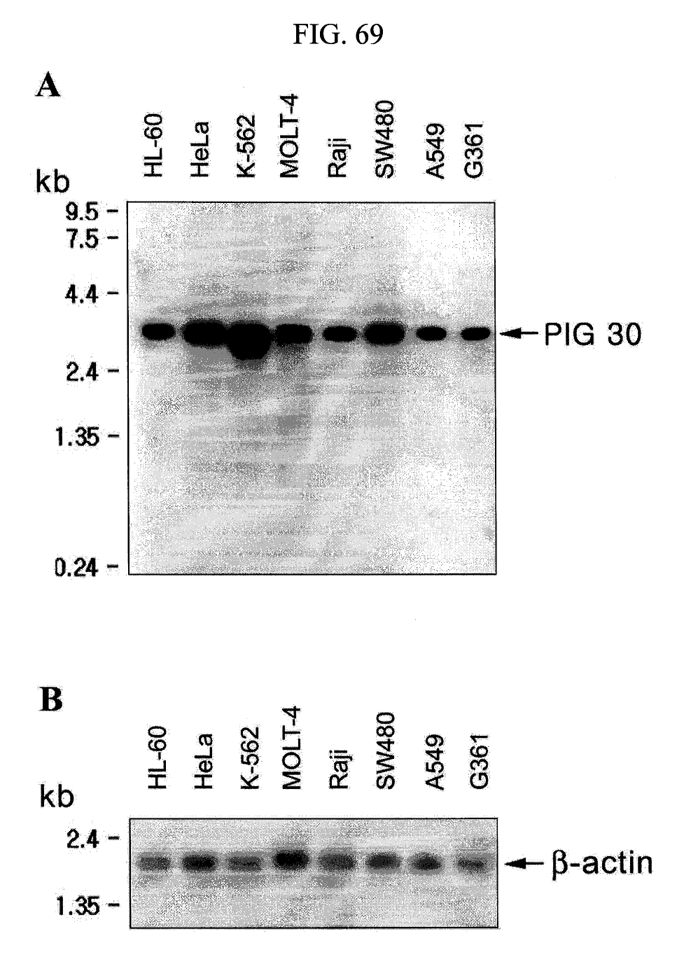

FIG. 69 shows a northern blotting result to determine whether or not the PIG30 protooncogene is expressed in the human cancer cell lines, for example HL-60, HeLa, K-562, MOLT-4, Raji, SW480, A549 and G361 (Clontech). A bottom of FIG. 69 shows the northern blotting result indicating whether or not β-actin mRNA is transcribed by hybridizing the same sample with β-actin probe. As shown in FIG. 69, it was revealed that the PIG30 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 28 shows a northern blotting result to determine whether or not the PIG31 protooncogene is expressed in the normal breast tissue, the breast cancer tissue and the breast cancer cell line (MCF-7). As shown in FIG. 28, it was revealed that the expression level of the PIG31 protooncogene was significantly increased in the breast cancer tissue and the breast cancer cell line MCF-7, but very low or not detected in the normal tissue. In FIG. 28, a lane “Normal” represents the normal breast tissue, a lane “Cancer” represents the breast cancer tissue, and a lane “MCF-7” represents the breast cancer cell line. A bottom of FIG. 28 shows the northern blotting result indicating whether or not β-actin mRNA is transcribed by hybridizing the same sample with β-actin probe.

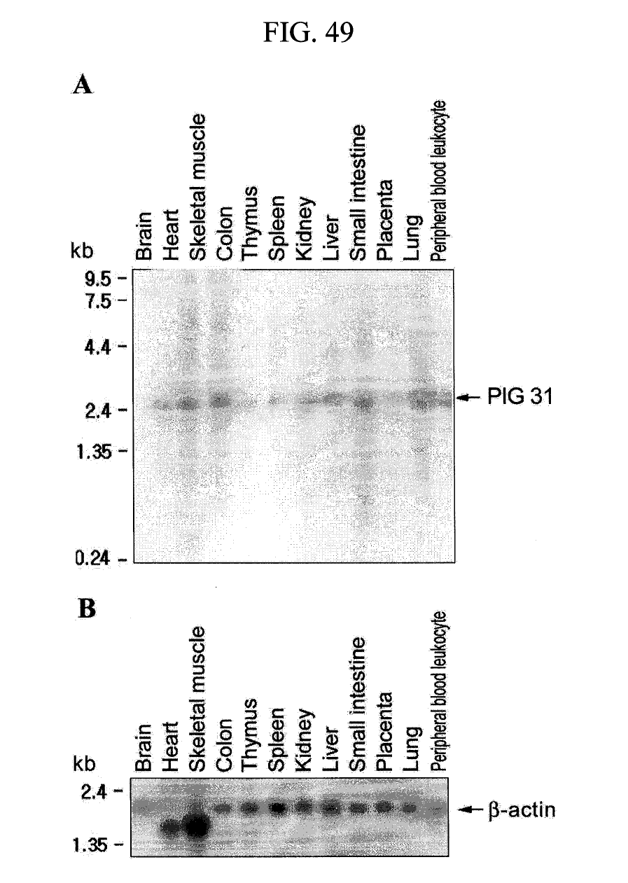

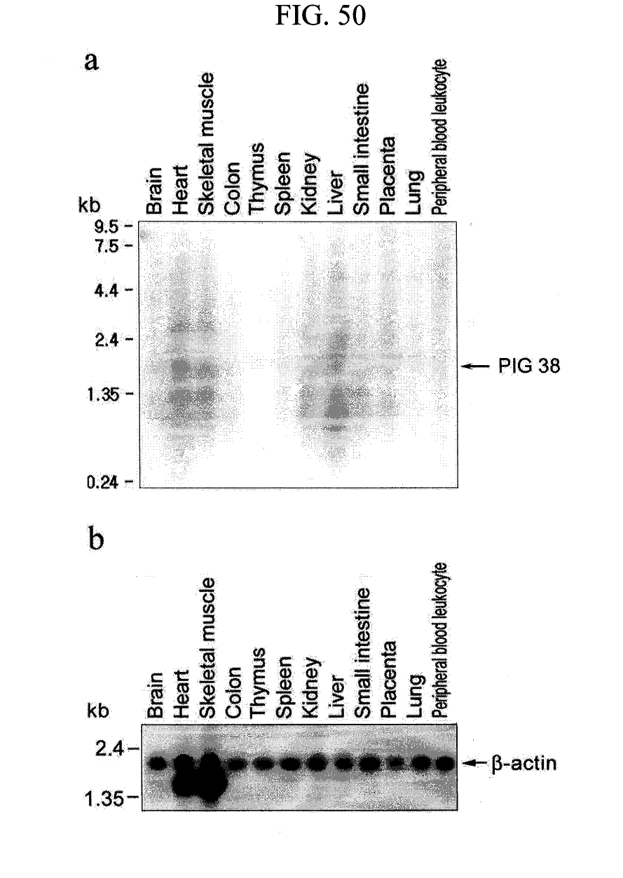

FIG. 49 shows a northern blotting result to determine whether or not the PIG31 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 49 shows the northern blotting result indicating whether or not β-actin mRNA is transcribed by hybridizing the same sample with β-actin probe. As shown in FIG. 49, it was revealed that the PIG31 mRNA transcript (approximately 2.5 kb) was rarely expressed in the various normal tissues.

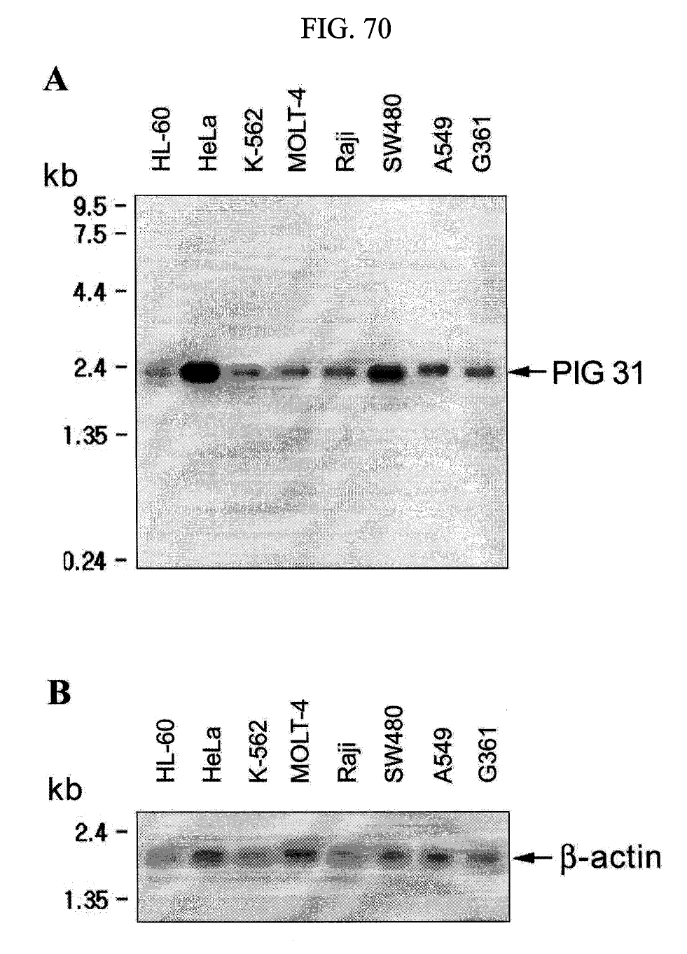

FIG. 70 shows a northern blotting result to determine whether or not the PIG31 protooncogene is expressed in the human cancer cell lines, for example HL-60, HeLa, K-562, MOLT-4, Raji, SW480, A549 and G361 (Clontech). A bottom of FIG. 70 shows the northern blotting result indicating whether or not β-actin mRNA is transcribed by hybridizing the same sample with β-actin probe. As shown in FIG. 70, it was revealed that the PIG31 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

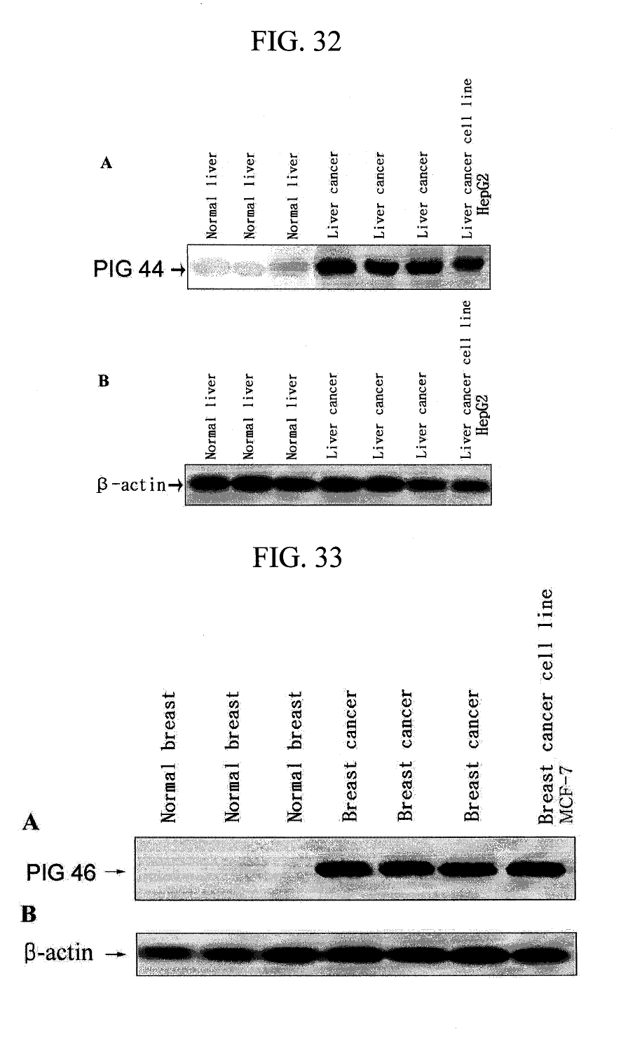

FIG. 33 shows a northern blotting result to determine whether or not the PIG46 protooncogene is expressed in the normal breast tissue, the breast cancer tissue and the breast cancer cell line (MCF-7). As shown in FIG. 33, it was revealed that the expression level of the PIG46 protooncogene was significantly increased in the breast cancer tissue and the breast cancer cell line MCF-7, but very low or not detected in the normal tissue. In FIG. 33, a lane “Normal” represents the normal breast tissue, a lane “Cancer” represents the breast cancer tissue, and a lane “MCF-7” represents the breast cancer cell line. A bottom of FIG. 33 shows the northern blotting result indicating whether or not β-actin mRNA is transcribed by hybridizing the same sample with β-actin probe.

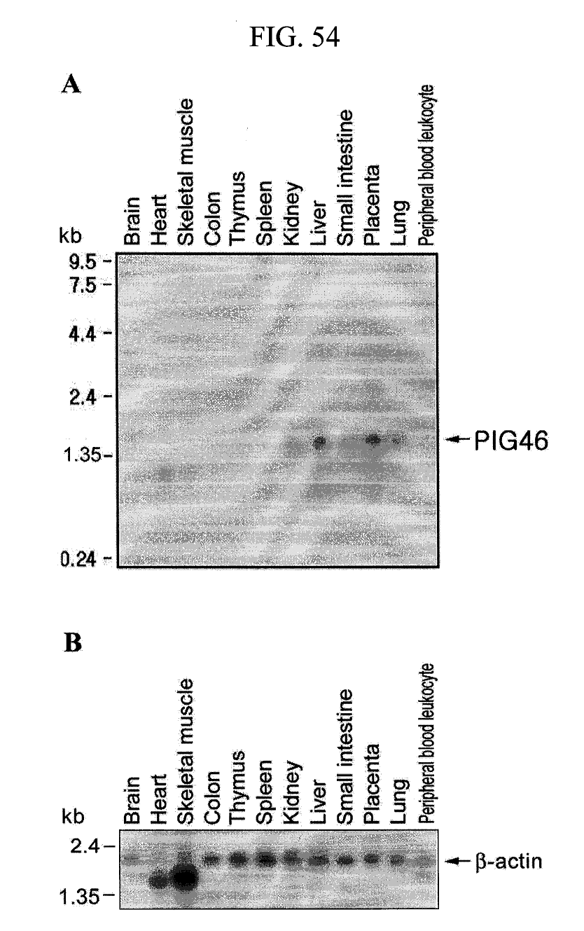

FIG. 54 shows a northern blotting result to determine whether or not the PIG46 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 54 shows the northern blotting result indicating whether or not β-actin mRNA is transcribed by hybridizing the same sample with β-actin probe. As shown in FIG. 54, it was revealed that the PIG46 mRNA transcript (approximately 1.4 kb) was very rarely expressed or not expressed in the various normal tissues.

FIG. 75 shows a northern blotting result to determine whether or not the PIG46 protooncogene is expressed in the human cancer cell lines, for example HL-60, HeLa, K-562, MOLT-4, Raji, SW480, A549 and G361 (Clontech). A bottom of FIG. 75 shows the northern blotting result indicating whether or not β-actin mRNA is transcribed by hybridizing the same sample with β-actin probe. As shown in FIG. 75, it was revealed that the PIG46 mRNA transcript (approximately 1.4 kb) was highly expressed in the HeLa uterine cancer cell line, the chronic myelogenous leukemia cell line K-562, the colon cancer cell line SW480 and the lung cancer cell line A549, but not expressed in the promyelocyte leukemia cell line HL-60, the lymphoblastic leukaemia cell line MOLT-4, the Burkitt lymphoma cell line Raji and the skin cancer cell line G361.

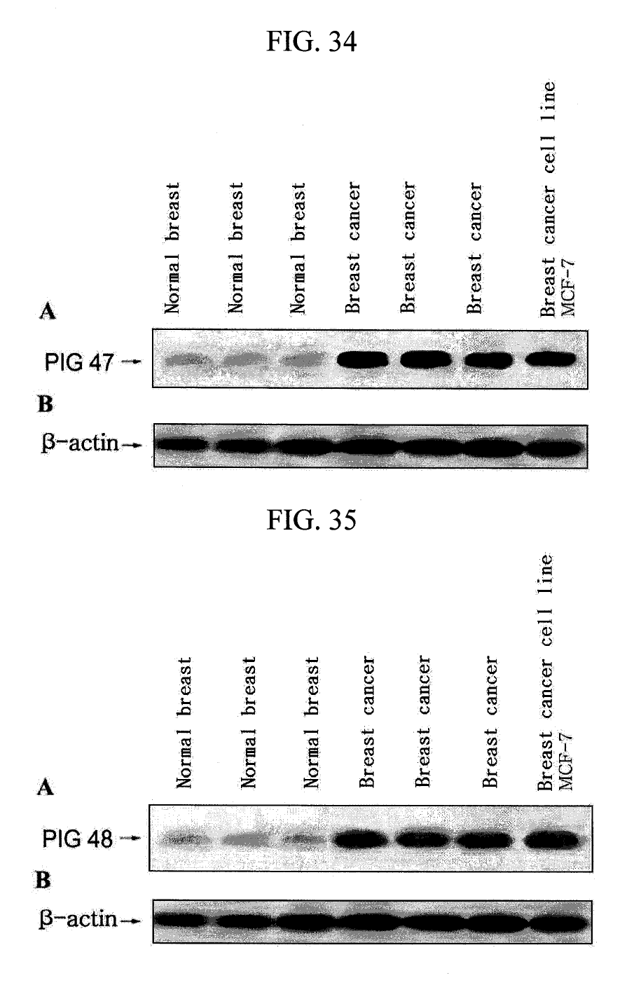

FIG. 34 shows a northern blotting result to determine whether or not the PIG47 protooncogene is expressed in the normal breast tissue, the breast cancer tissue and the breast cancer cell line (MCF-7). As shown in FIG. 34, it was revealed that the expression level of the PIG47 protooncogene was significantly increased in the breast cancer tissue and the breast cancer cell line MCF-7, but very low or not detected in the normal tissue. In FIG. 34, a lane “Normal” represents the normal breast tissue, a lane “Cancer” represents the breast cancer tissue, and a lane “MCF-7” represents the breast cancer cell line. A bottom of FIG. 34 shows the northern blotting result indicating whether or not β-actin mRNA is transcribed by hybridizing the same sample with β-actin probe.

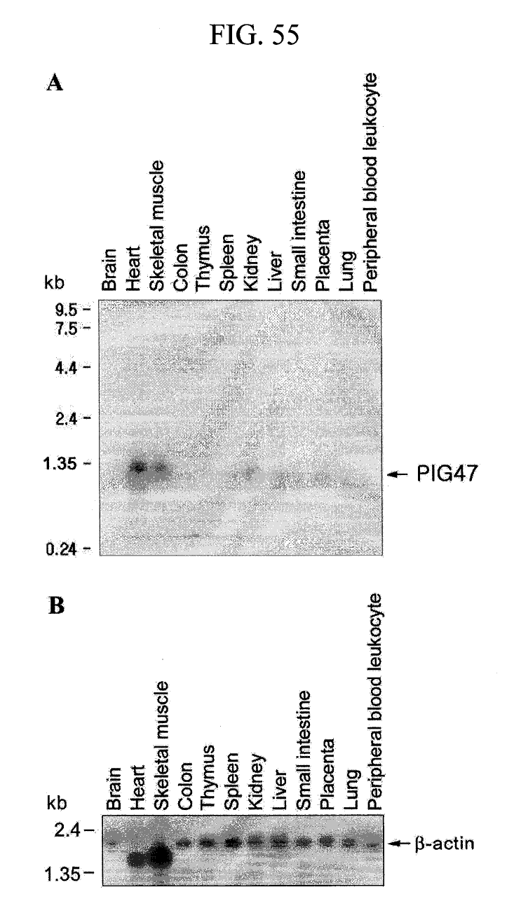

FIG. 55 shows a northern blotting result to determine whether or not the PIG47 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 55 shows the northern blotting result indicating whether or not β-actin mRNA is transcribed by hybridizing the same sample with β-actin probe. As shown in FIG. 55, it was revealed that the PIG47 mRNA transcript (approximately 1.3 kb) was expressed in the normal heart and muscle tissues, but very rarely expressed in the various normal tissues.

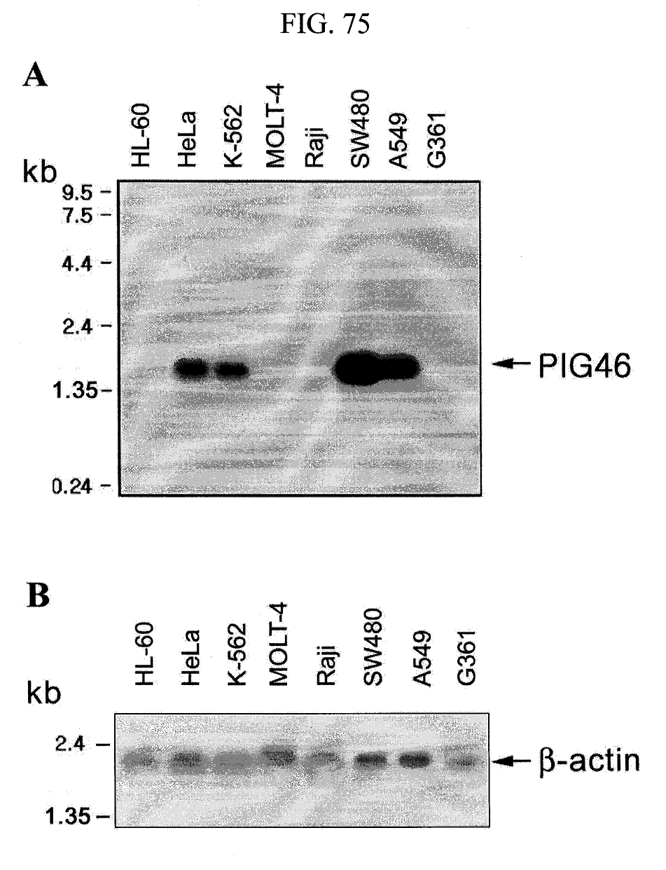

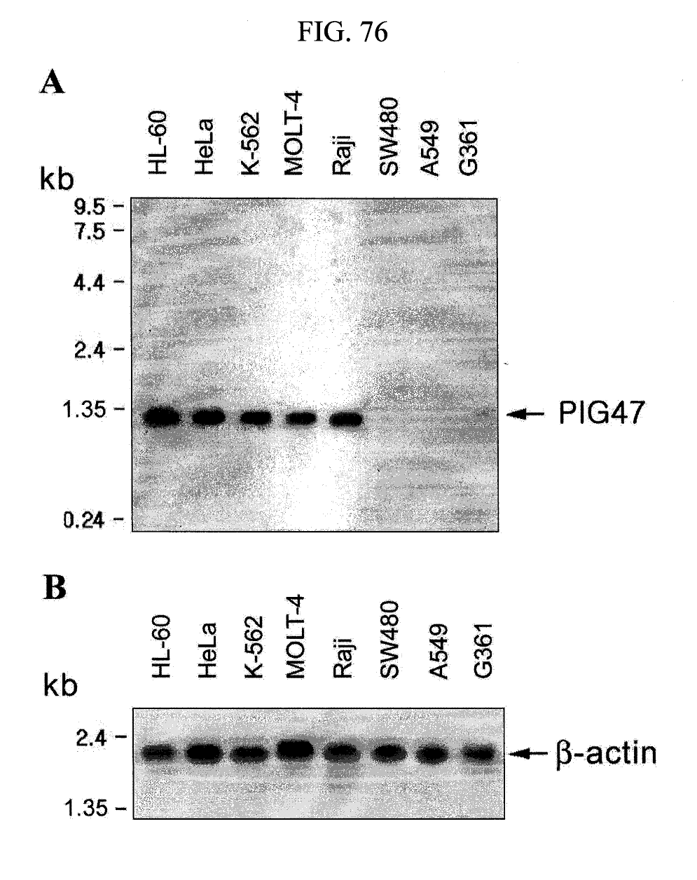

FIG. 76 shows a northern blotting result to determine whether or not the PIG47 protooncogene is expressed in the human cancer cell lines, for example HL-60, HeLa, K-562, MOLT-4, Raji, SW480, A549 and G361 (Clontech). A bottom of FIG. 76 shows the northern blotting result indicating whether or not β-actin mRNA is transcribed by hybridizing the same sample with β-actin probe. As shown in FIG. 76, it was revealed that the PIG47 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4 and the Burkitt lymphoma cell line Raji, but not expressed in the colon cancer cell line SW480, the lung cancer cell line A549 and the skin cancer cell line G361.

FIG. 35 shows a northern blotting result to determine whether or not the PIG48 protooncogene is expressed in the normal breast tissue, the breast cancer tissue and the breast cancer cell line (MCF-7). As shown in FIG. 35, it was revealed that the expression level of the PIG48 protooncogene was significantly increased in the breast cancer tissue and the breast cancer cell line MCF-7, but very low or not detected in the normal tissue. In FIG. 35, a lane “Normal” represents the normal breast tissue, a lane “Cancer” represents the breast cancer tissue, and a lane “MCF-7” represents the breast cancer cell line. A bottom of FIG. 35 shows the northern blotting result indicating whether or not β-actin mRNA is transcribed by hybridizing the same sample with β-actin probe.

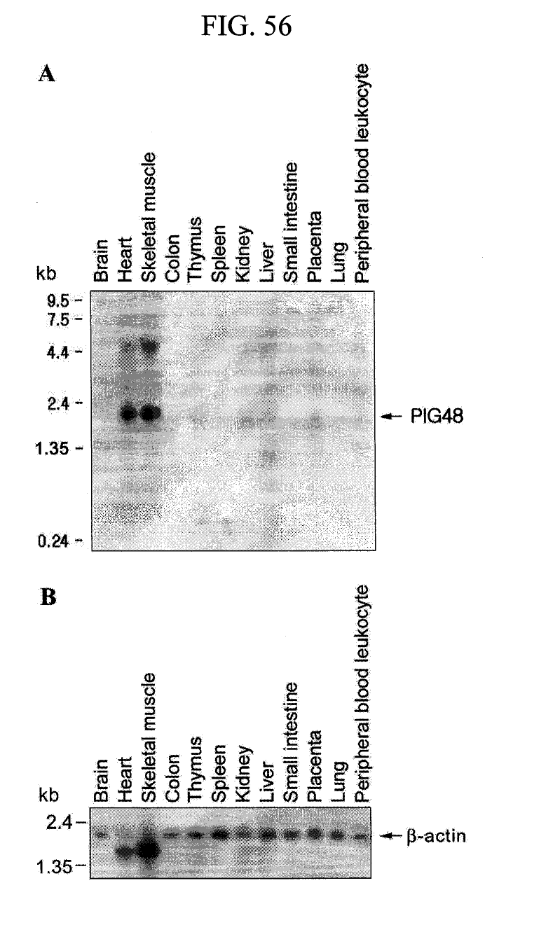

FIG. 56 shows a northern blotting result to determine whether or not the PIG48 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 56 shows the northern blotting result indicating whether or not β-actin mRNA is transcribed by hybridizing the same sample with β-actin probe. As shown in FIG. 56, it was revealed that the PIG48 mRNA transcript (approximately 2.0 kb) was very rarely expressed or not expressed in the various normal tissues.

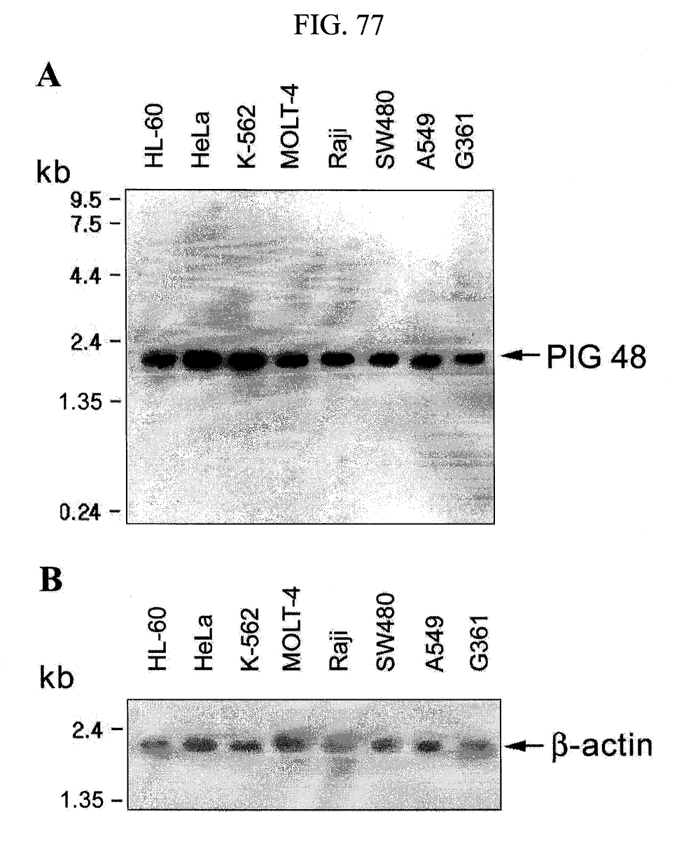

FIG. 77 shows a northern blotting result to determine whether or not the PIG48 protooncogene is expressed in the human cancer cell lines, for example HL-60, HeLa, K-562, MOLT-4, Raji, SW480, A549 and G361 (Clontech). A bottom of FIG. 77 shows the northern blotting result indicating whether or not β-actin mRNA is transcribed by hybridizing the same sample with β-actin probe. As shown in FIG. 77, it was revealed that the PIG48 mRNA transcript was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

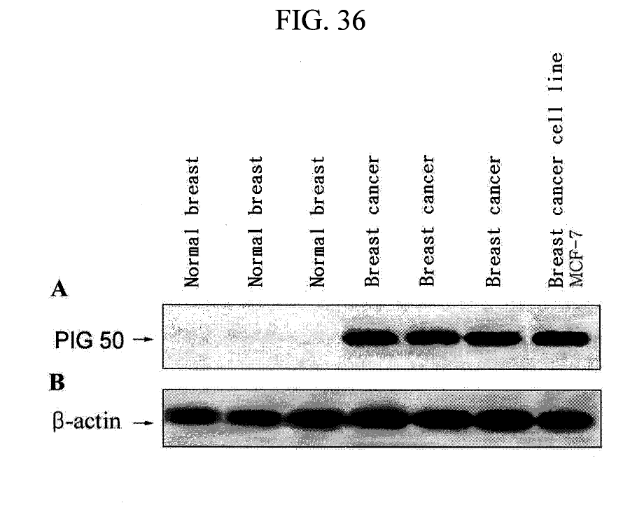

FIG. 36 shows a northern blotting result to determine whether or not the PIG50 protooncogene is expressed in the normal breast tissue, the breast cancer tissue and the breast cancer cell line (MCF-7). As shown in FIG. 36, it was revealed that the expression level of the PIG50 protooncogene was significantly increased in the breast cancer tissue and the breast cancer cell line MCF-7, but not detected in the normal tissue. In FIG. 36, a lane “Normal” represents the normal breast tissue, a lane “Cancer” represents the breast cancer tissue, and a lane “MCF-7” represents the breast cancer cell line. A bottom of FIG. 36 shows the northern blotting result indicating whether or not β-actin mRNA is transcribed by hybridizing the same sample with β-actin probe.

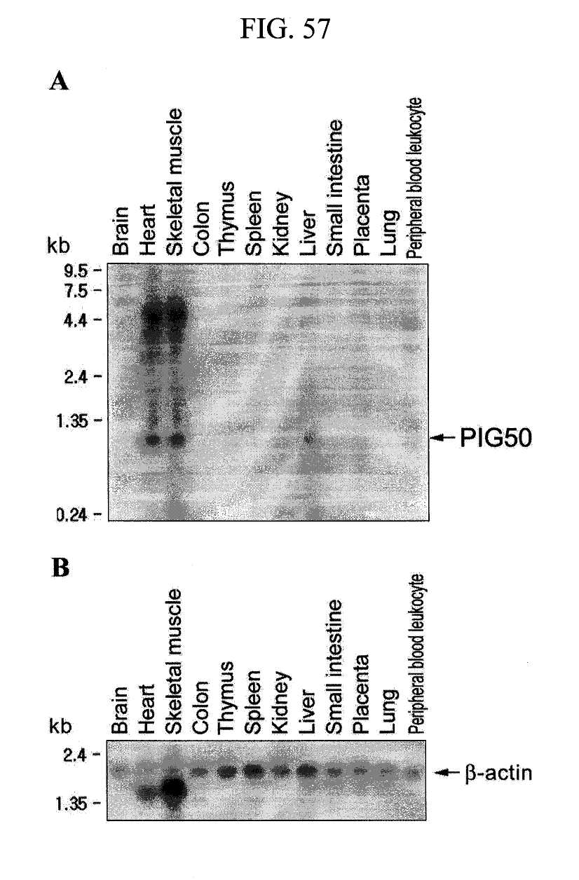

FIG. 57 shows a northern blotting result to determine whether or not the PIG50 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 57 shows the northern blotting result indicating whether or not β-actin mRNA is transcribed by hybridizing the same sample with β-actin probe. As shown in FIG. 57, it was revealed that the PIG50 mRNA transcript (approximately 1.0 kb) was very rarely expressed or not expressed in the various normal tissues. Also, it was revealed that a PIG50 mRNA transcript having a size of approximately 5.0 kb was very rarely expressed at the same time.

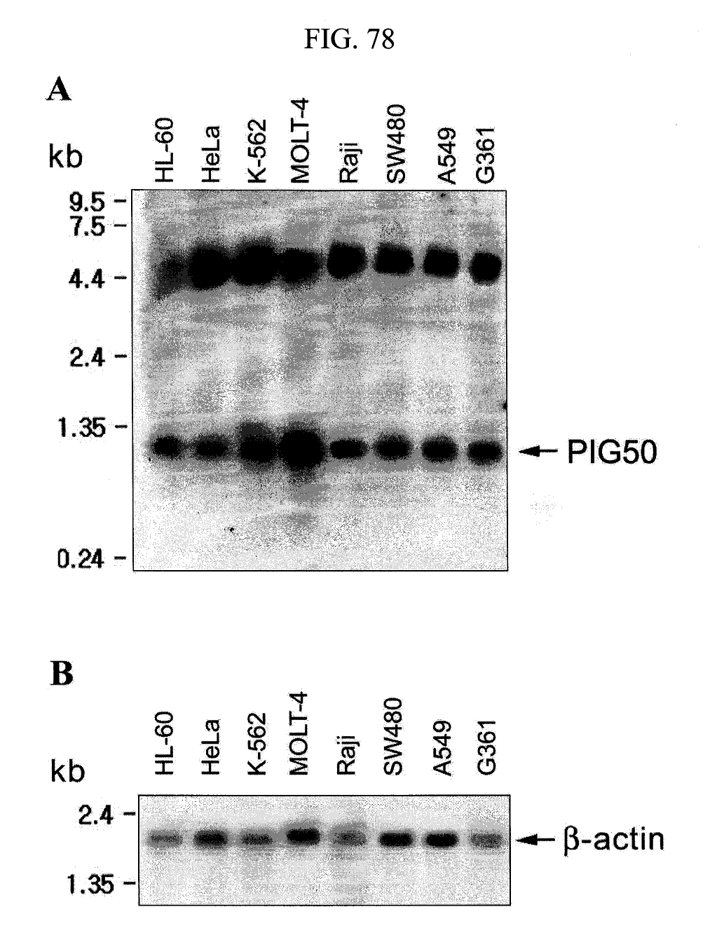

FIG. 78 shows a northern blotting result to determine whether or not the PIG50 protooncogene is expressed in the human cancer cell lines, for example HL-60, HeLa, K-562, MOLT-4, Raji, SW480, A549 and G361 (Clontech). A bottom of FIG. 78 shows the northern blotting result indicating whether or not β-actin mRNA is transcribed by hybridizing the same sample with β-actin probe. As shown in FIG. 78, it was revealed that the PIG50 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that a PIG50 mRNA transcript having a size of approximately 5.0 kb was highly expressed at the same time.

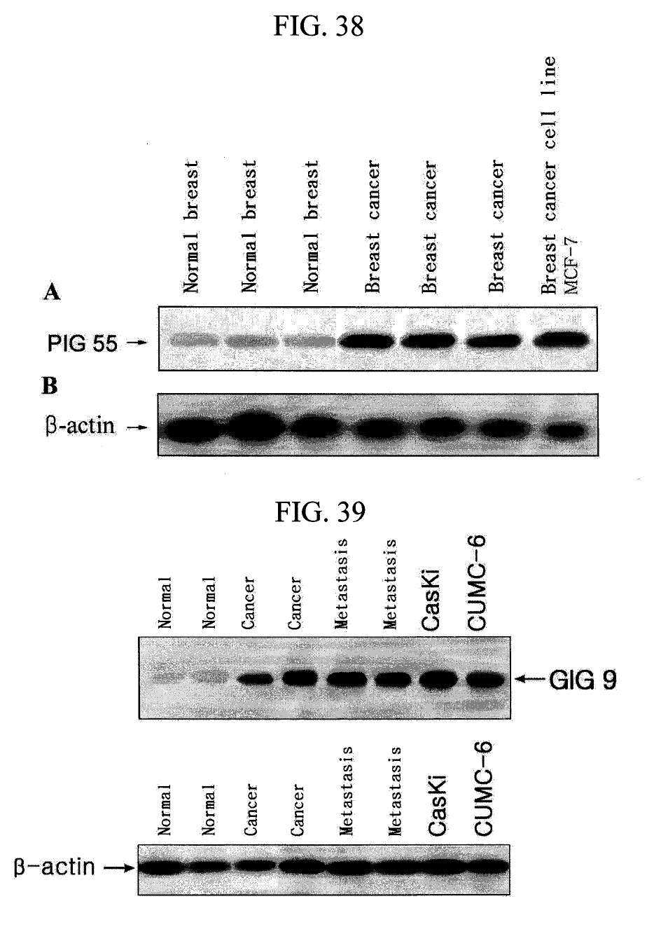

FIG. 38 shows a northern blotting result to determine whether or not the PIG55 protooncogene is expressed in the normal breast tissue, the breast cancer tissue and the breast cancer cell line (MCF-7). As shown in FIG. 38, it was revealed that the expression level of the PIG55 protooncogene was significantly increased in the breast cancer tissue and the breast cancer cell line MCF-7, but very low or not detected in the normal tissue. In FIG. 38, a lane “Normal” represents the normal breast tissue, a lane “Cancer” represents the breast cancer tissue, and a lane “MCF-7” represents the breast cancer cell line. A bottom of FIG. 38 shows the northern blotting result indicating whether or not β-actin mRNA is transcribed by hybridizing the same sample with β-actin probe.

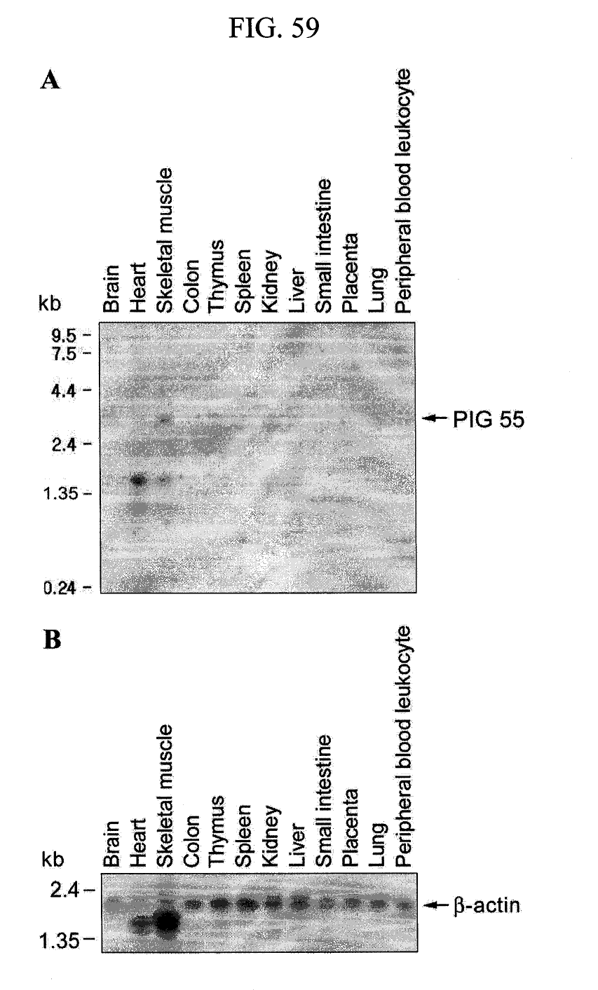

FIG. 59 shows a northern blotting result to determine whether or not the PIG55 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 59 shows the northern blotting result indicating whether or not β-actin mRNA is transcribed by hybridizing the same sample with β-actin probe. As shown in FIG. 59, it was revealed that the PIG55 mRNA transcript (approximately 3.0 kb) was rarely expressed or not expressed in the various normal tissues.

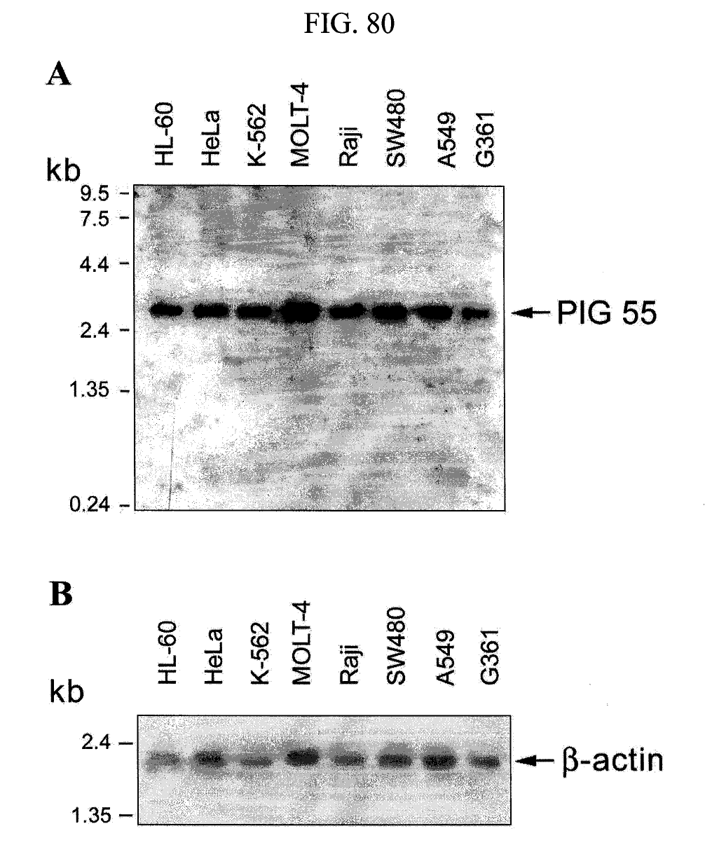

FIG. 80 shows a northern blotting result to determine whether or not the PIG55 protooncogene is expressed in the human cancer cell lines, for example HL-60, HeLa, K-562, MOLT-4, Raji, SW480, A549 and G361 (Clontech). A bottom of FIG. 80 shows the northern blotting result indicating whether or not β-actin mRNA is transcribed by hybridizing the same sample with β-actin probe. As shown in FIG. 80, it was revealed that the PIG55 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

7-2. PIG18, PIG23, PIG27 PIG28 and GIG9

The total RNA samples were extracted from the normal exocervical tissue, the cervical cancer tissue, the metastatic cervical lymph node tissue and the cervical cancer cell lines CaSki (ATCC CRL 1550) and CUMC-6 in the same manner as in Example 1-2.

In order to determine an expression level of each of the PIG or GIG genes, 20 μg of each of the total denatured RNA samples extracted from the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed full-length PIG23 cDNA probe prepared using the Rediprime II random prime labelling system ((Amersham, United Kingdom). The northern blotting analysis was repeated twice, and then the resultant blots were quantitified with the densitometer and normalized with the β-actin.

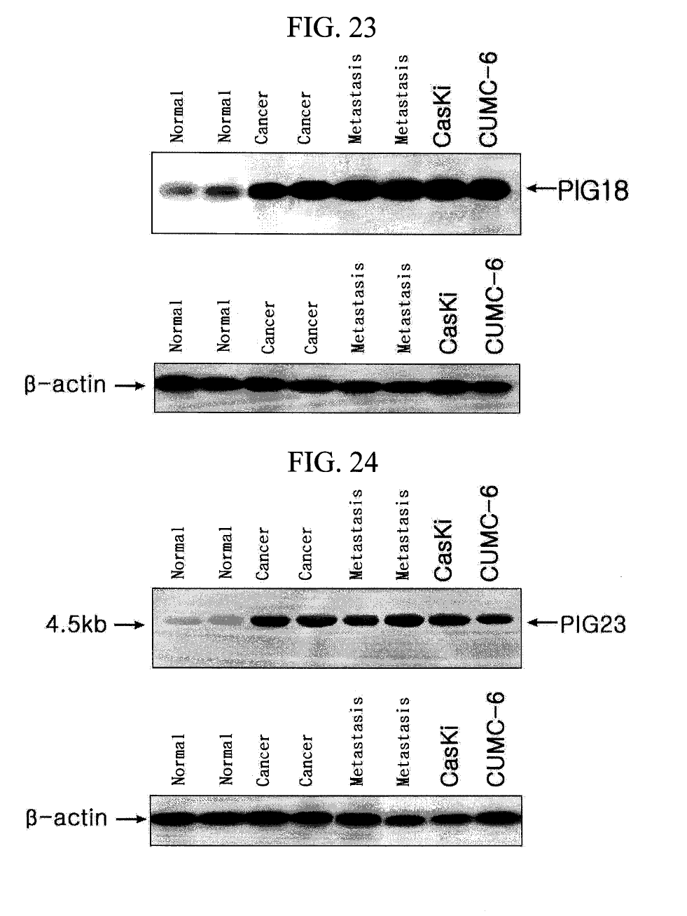

FIG. 23 shows a northern blotting result to determine whether or not the PIG18 protooncogene is expressed in the normal exocervical tissue, the cervical cancer tissue, the metastatic cervical lymph node tissue and the cervical cancer cell lines (CaSki and CUMC-6). As shown in FIG. 23, it was revealed that the expression level of the PIG18 protooncogene was increased, that is, a dominant PIG18 mRNA transcript having a size of approximately 5.0 kb was overexpressed in the cervical cancer tissue and the cervical cancer cell lines CaSki and CUMC-6. In FIG. 23, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 23 shows the northern blotting result indicating whether or not β-actin mRNA is transcribed by hybridizing the same sample with β-actin probe.

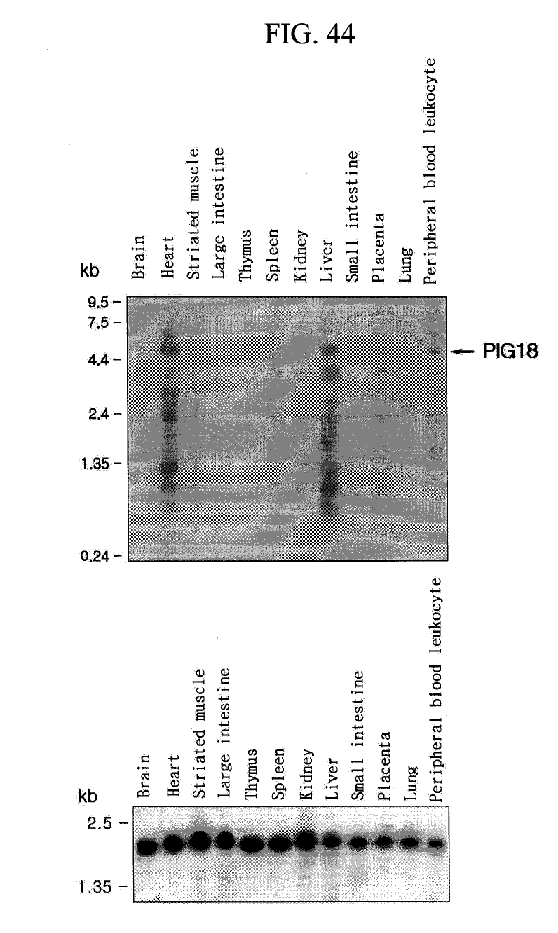

FIG. 44 shows a northern blotting result to determine whether or not the PIG18 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 44 shows the northern blotting result indicating whether or not β-actin mRNA is transcribed by hybridizing the same sample with β-actin probe. As shown in FIG. 44, it was revealed that a PIG18 mRNA transcript (the dominant PIG18 mRNA transcript having a size of approximately 5.0 kb) was very rarely expressed or not expressed in the normal tissues such as the brain, the heart, the muscle, the large intestine, the thymus, the spleen, the kidney, the liver, the small intestine, the placenta, the lung and the peripheral blood leukocyte. Also, it was revealed that a PIG18 mRNA transcript having a size of approximately 3.0 kb was very rarely expressed in the normal heart at the same time.

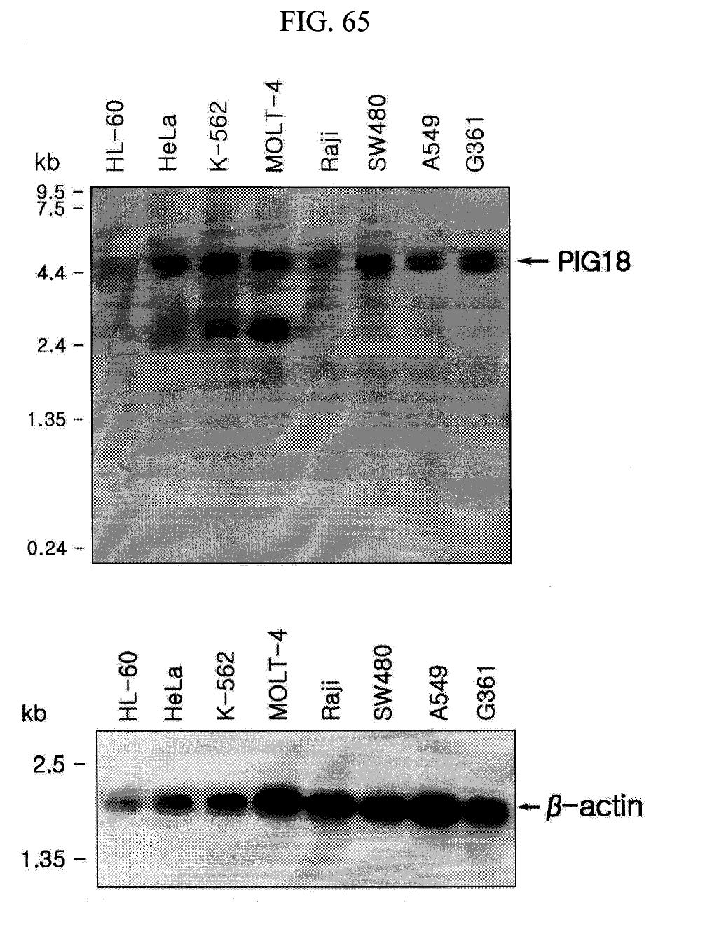

FIG. 65 shows a northern blotting result to determine whether or not the PIG18 protooncogene is expressed in the human cancer cell lines, for example HL-60, HeLa, K-562, MOLT-4, Raji, SW480, A549 and G361 (Clontech). A bottom of FIG. 65 shows the northern blotting result indicating whether or not β-actin mRNA is transcribed by hybridizing the same sample with β-actin probe. As shown in FIG. 65, it was revealed that a PIG18 mRNA transcript (the dominant PIG18 mRNA transcript having a size of approximately 5.0 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that a PIG18 mRNA transcript having a size of approximately 3.0 kb was simultaneously expressed at an increased level in the HeLa uterine cancer cell line, the chronic myelogenous leukemia cell line K-562 and the lymphoblastic leukaemia cell line MOLT-4.

FIG. 24 shows a northern blotting result to determine whether or not the PIG23 protooncogene is expressed in the normal exocervical tissue, the cervical cancer tissue, the metastatic cervical lymph node tissue and the cervical cancer cell lines (CaSki and CUMC-6). As shown in FIG. 24, it was revealed that the expression level of the PIG23 protooncogene was increased, that is, a dominant PIG23 mRNA transcript having a size of approximately 4.5 kb was overexpressed in the cervical cancer tissue, the metastatic cervical lymph node tissue and the cervical cancer cell lines CaSki and CUMC-6. In FIG. 24, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 24 shows the northern blotting result indicating whether or not β-actin mRNA is transcribed by hybridizing the same sample with β-actin probe.

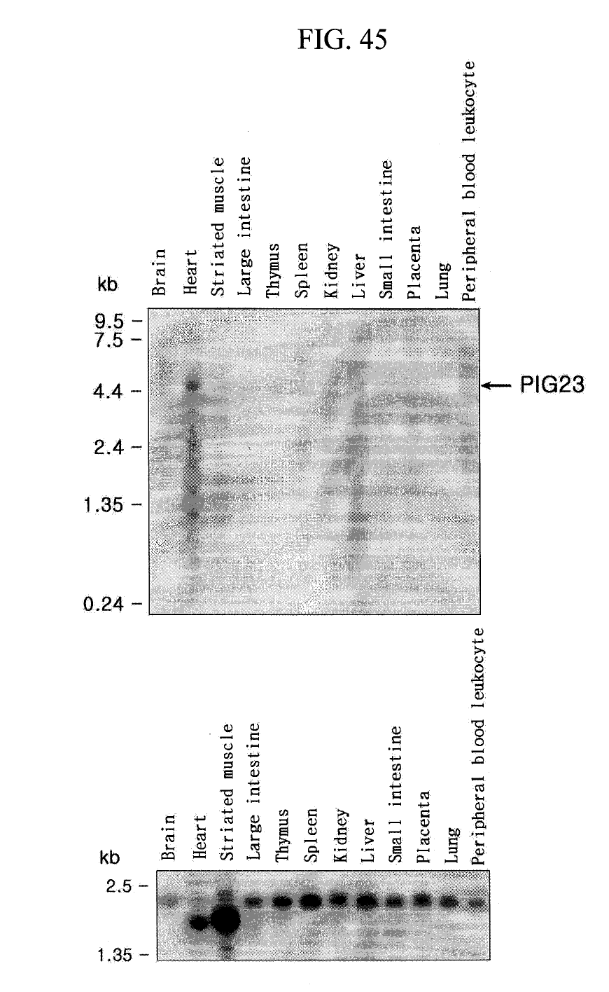

FIG. 45 shows a northern blotting result to determine whether or not the PIG23 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 45 shows the northern blotting result indicating whether or not β-actin mRNA is transcribed by hybridizing the same sample with β-actin probe. As shown in FIG. 45, it was revealed that a PIG23 mRNA transcript (the dominant PIG18 mRNA transcript having a size of approximately 4.5 kb) was very rarely expressed or not expressed in the normal tissues such as the brain, the heart, the muscle, the large intestine, the thymus, the spleen, the kidney, the liver, the small intestine, the placenta, the lung and the peripheral blood leukocyte.

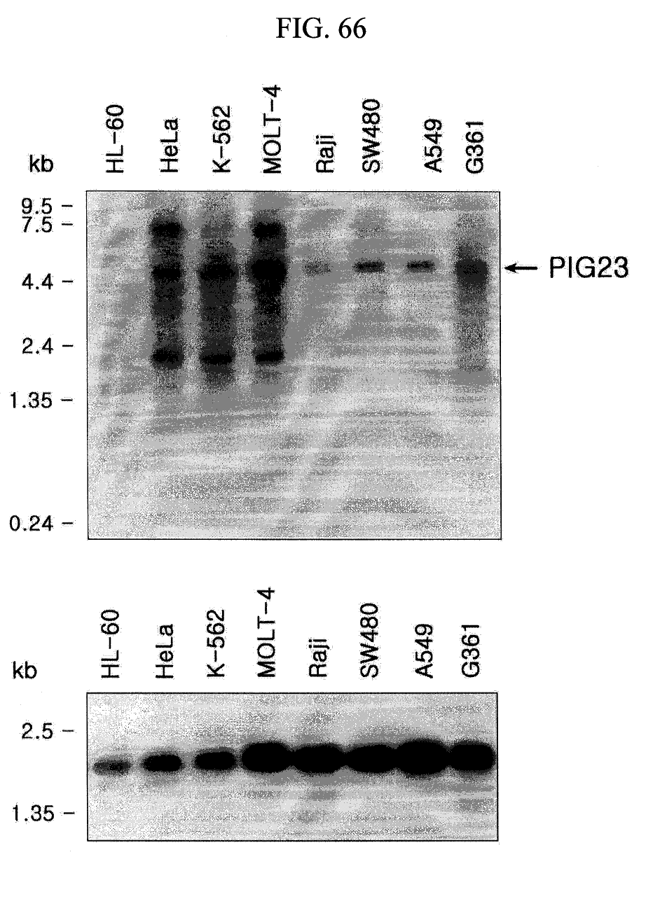

FIG. 66 shows a northern blotting result to determine whether or not the PIG23 protooncogene is expressed in the human cancer cell lines, for example HL-60, HeLa, K-562, MOLT-4, Raji, SW480, A549 and G361 (Clontech). A bottom of FIG. 66 shows the northern blotting result indicating whether or not β-actin mRNA is transcribed by hybridizing the same sample with β-actin probe. As shown in FIG. 66, it was revealed that a PIG23 mRNA transcript (the dominant PIG23 mRNA transcript having a size of approximately 4.5 kb) was very highly expressed in the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that PIG23 mRNA transcripts having sizes of approximately 7.0 kb and 2.0 kb were simultaneously expressed at an increased level in the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

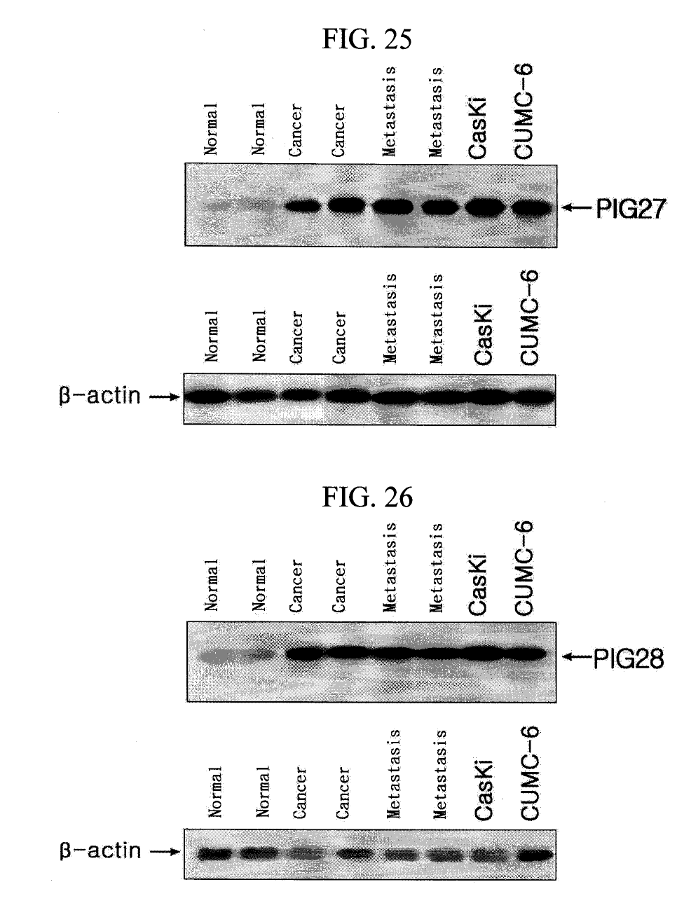

FIG. 25 shows a northern blotting result to determine whether or not the PIG27 protooncogene is expressed in the normal exocervical tissue, the cervical cancer tissue, the metastatic cervical lymph node tissue and the cervical cancer cell lines (CaSki and CUMC-6). As shown in FIG. 25, it was revealed that the expression level of the PIG27 protooncogene was increased, that is, a dominant PIG27 mRNA transcript having a size of approximately 1.5 kb was overexpressed in the cervical cancer tissue and the cervical cancer cell lines CaSki and CUMC-6. In FIG. 25, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 25 shows the northern blotting result indicating whether or not β-actin mRNA is transcribed by hybridizing the same sample with β-actin probe.

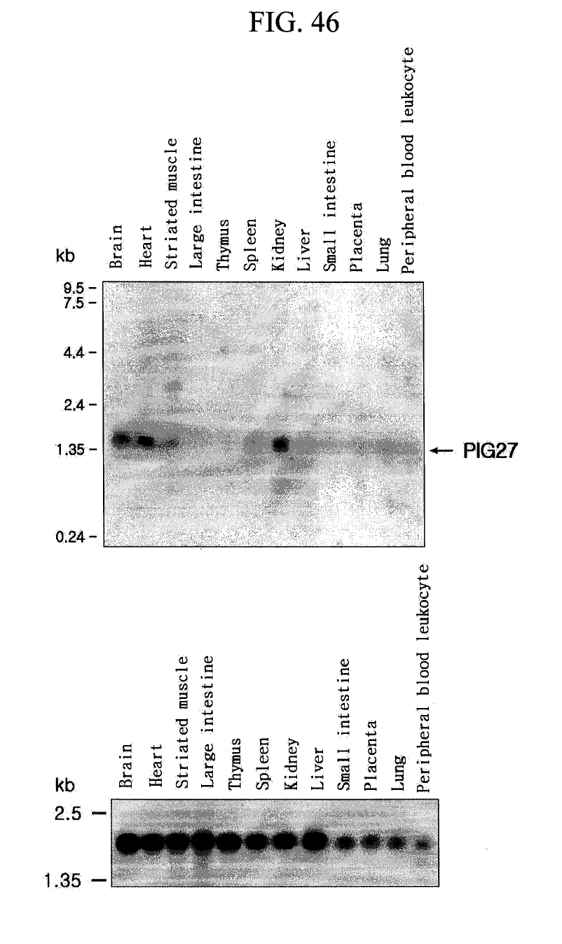

FIG. 46 shows a northern blotting result to determine whether or not the PIG27 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 46 shows the northern blotting result indicating whether or not β-actin mRNA is transcribed by hybridizing the same sample with β-actin probe. As shown in FIG. 46, it was revealed that a PIG27 mRNA transcript (the dominant PIG27 mRNA transcript having a size of approximately 1.5 kb) was very rarely expressed in the normal tissues such as the brain, the heart, the muscle, the large intestine, the thymus, the spleen, the kidney, the liver, the small intestine, the placenta, the lung and the peripheral blood leukocyte.

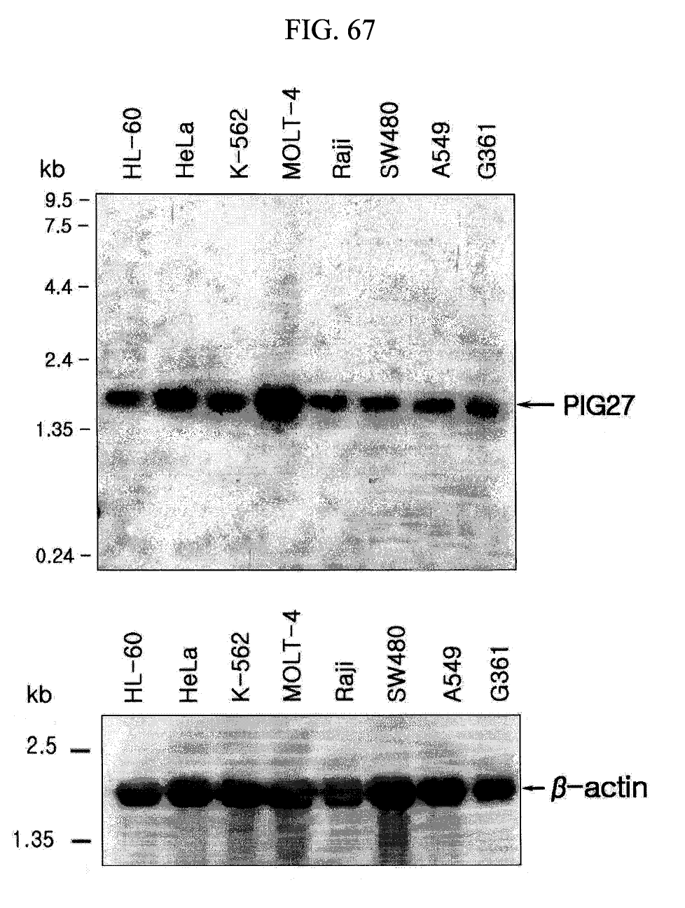

FIG. 67 shows a northern blotting result to determine whether or not the PIG27 protooncogene is expressed in the human cancer cell lines, for example HL-60, HeLa, K-562, MOLT-4, Raji, SW480, A549 and G361 (Clontech). A bottom of FIG. 67 shows the northern blotting result indicating whether or not β-actin mRNA is transcribed by hybridizing the same sample with β-actin probe. As shown in FIG. 67, it was revealed that a PIG27 mRNA transcript (the dominant PIG27 mRNA transcript having a size of approximately 1.5 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 26 shows a northern blotting result to determine whether or not the PIG28 protooncogene is expressed in the normal exocervical tissue, the cervical cancer tissue, the metastatic cervical lymph node tissue and the cervical cancer cell lines (CaSki and CUMC-6). As shown in FIG. 26, it was revealed that the expression level of the PIG28 protooncogene was increased, that is, a dominant PIG28 mRNA transcript having a size of approximately 1.5 kb was overexpressed in the cervical cancer tissue and the cervical cancer cell lines CaSki and CUMC-6. In FIG. 26, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 26 shows the northern blotting result indicating whether or not β-actin mRNA is transcribed by hybridizing the same sample with β-actin probe.

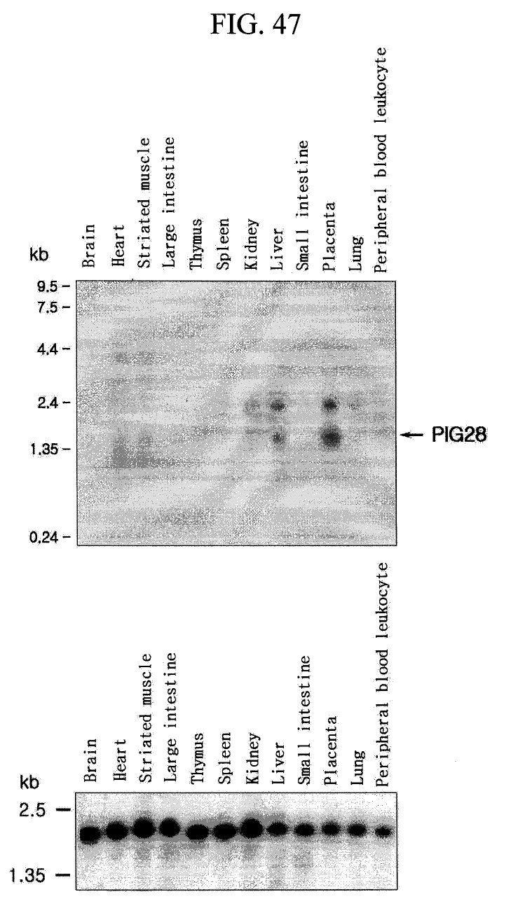

FIG. 47 shows a northern blotting result to determine whether or not the PIG28 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 47 shows the northern blotting result indicating whether or not β-actin mRNA is transcribed by hybridizing the same sample with β-actin probe. As shown in FIG. 47, it was revealed that a PIG28 mRNA transcript (the dominant PIG28 mRNA transcript having a size of approximately 1.5 kb) was very rarely expressed or not expressed in the normal tissues such as the brain, the heart, the muscle, the large intestine, the thymus, the spleen, the kidney, the liver, the small intestine, the placenta, the lung and the peripheral blood leukocyte. Also, it was revealed that a PIG28 mRNA transcript having a size of approximately 2.2 kb was very rarely expressed or not expressed in the normal tissues such as the brain, the heart, the muscle, the large intestine, the thymus, the spleen, the kidney, the liver, the small intestine, the placenta, the lung and the peripheral blood leukocyte at the same time.

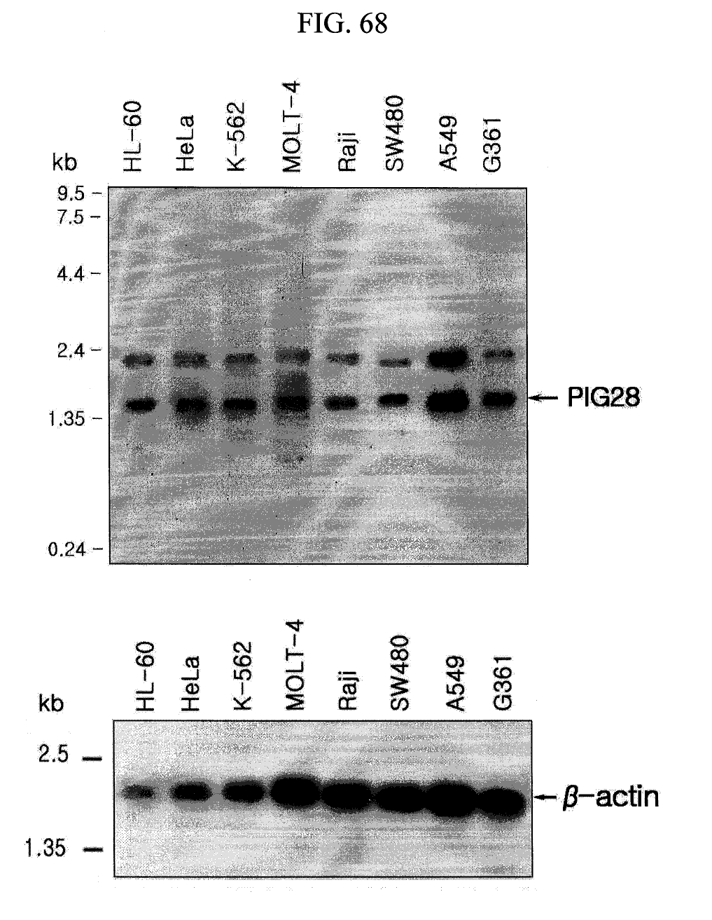

FIG. 68 shows a northern blotting result to determine whether or not the PIG28 protooncogene is expressed in the human cancer cell lines, for example HL-60, HeLa, K-562, MOLT-4, Raji, SW480, A549 and G361 (Clontech). A bottom of FIG. 68 shows the northern blotting result indicating whether or not β-actin mRNA is transcribed by hybridizing the same sample with β-actin probe. As shown in FIG. 68, it was revealed that a PIG28 mRNA transcript (the dominant PIG28 mRNA transcript having a size of approximately 1.5 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that a PIG28 mRNA transcript having a size of approximately 2.2 kb was simultaneously expressed at an increased level in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 39 shows a northern blotting result to determine whether or not the GIG9 protooncogene is expressed in the normal exocervical tissue, the cervical cancer tissue, the metastatic cervical lymph node tissue and the cervical cancer cell lines (CaSki and CUMC-6). As shown in FIG. 39, it was revealed that the expression level of the GIG9 protooncogene was increased, that is, a dominant GIG9 mRNA transcript having a size of approximately 1.5 kb was overexpressed in the cervical cancer tissue and the cervical cancer cell lines CaSki and CUMC-6. In FIG. 39, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 39 shows the northern blotting result indicating whether or not β-actin mRNA is transcribed by hybridizing the same sample with β-actin probe.

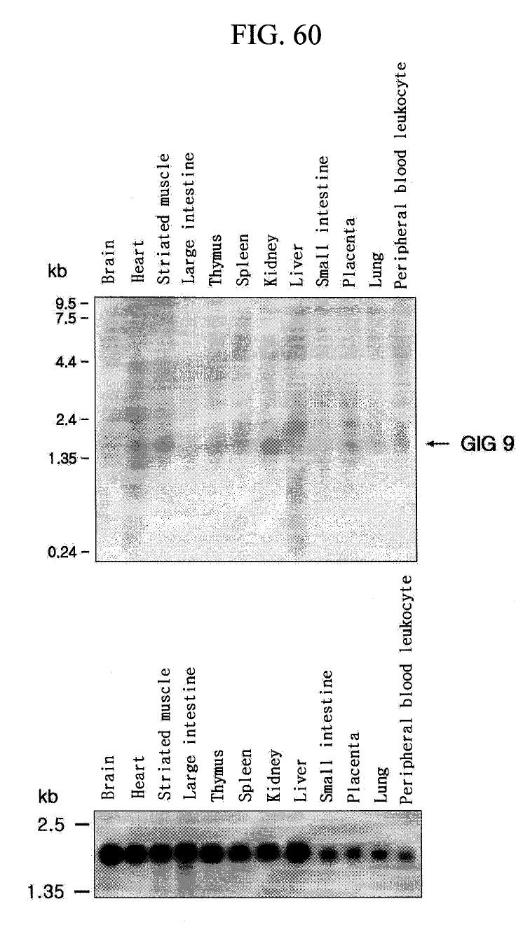

FIG. 60 shows a northern blotting result to determine whether or not the GIG9 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 60 shows the northern blotting result indicating whether or not β-actin mRNA is transcribed by hybridizing the same sample with β-actin probe. As shown in FIG. 60, it was revealed that a GIG9 mRNA transcript (the dominant GIG9 mRNA transcript having a size of approximately 1.5 kb) was very rarely expressed in the normal tissues such as the brain, the heart, the muscle, the large intestine, the thymus, the spleen, the kidney, the liver, the small intestine, the placenta, the lung and the peripheral blood leukocyte.

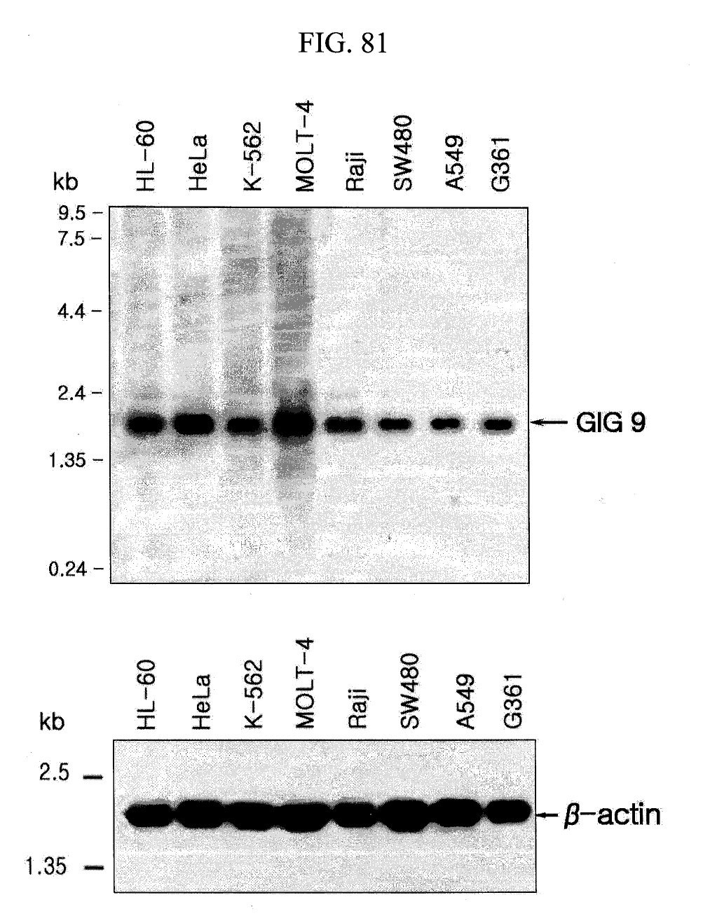

FIG. 81 shows a northern blotting result to determine whether or not the GIG9 protooncogene is expressed in the human cancer cell lines, for example HL-60, HeLa, K-562, MOLT-4, Raji, SW480, A549 and G361 (Clontech). A bottom of FIG. 81 shows the northern blotting result indicating whether or not β-actin mRNA is transcribed by hybridizing the same sample with β-actin probe. As shown in FIG. 81, it was revealed that a GIG9 mRNA transcript (the dominant GIG9 mRNA transcript having a size of approximately 1.5 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

7-3. PIG38, PIG43, PIG44. PIG54 and GIG18

The total RNA samples were extracted from the normal liver tissue, the liver cancer tissue and the liver cancer cell line HepG2 in the same manner as in Example 1-3.

In order to determine an expression level of each of the PIG genes, 20 μg of each of the total denatured RNA samples extracted from the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed HP103 cDNA probe prepared using the Rediprime II random prime labelling system ((Amersham, United Kingdom). The northern blotting analysis was repeated twice, and then the resultant blots were quantitified with the densitometer and normalized with the β-actin.

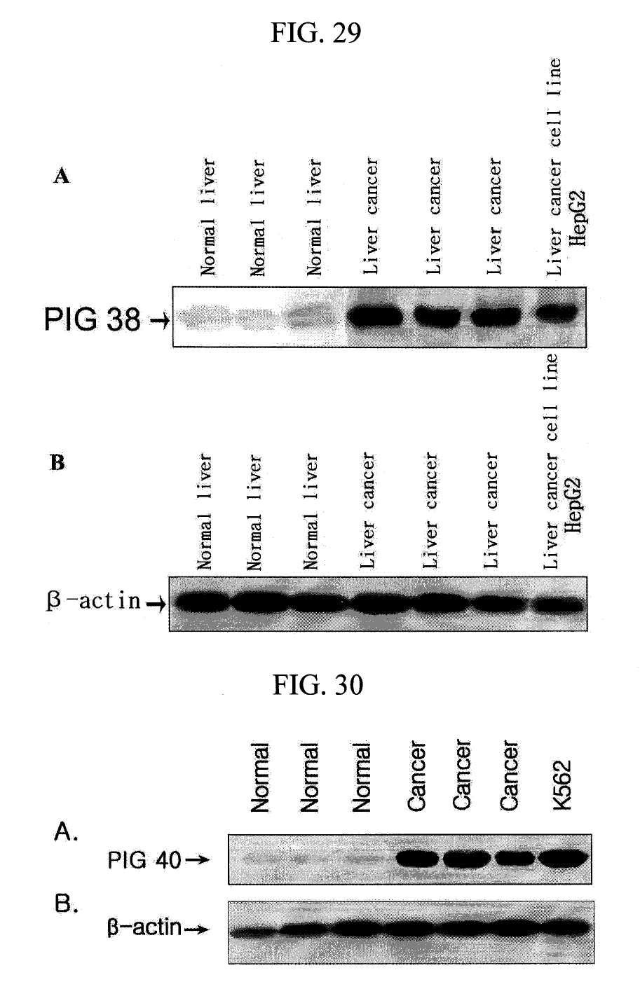

FIG. 29 shows a northern blotting result to determine whether or not the PIG38 protooncogene is expressed in the normal liver tissue, the liver cancer tissue and the liver cancer cell line (HepG2). As shown in FIG. 29, it was revealed that the PIG38 protooncogene was highly expressed in the liver cancer tissue and the liver cancer cell line HepG2, but not expressed or rarely expressed in the normal tissues. A bottom of FIG. 29 shows the northern blotting result indicating whether or not β-actin mRNA is transcribed by hybridizing the same sample with β-actin probe.

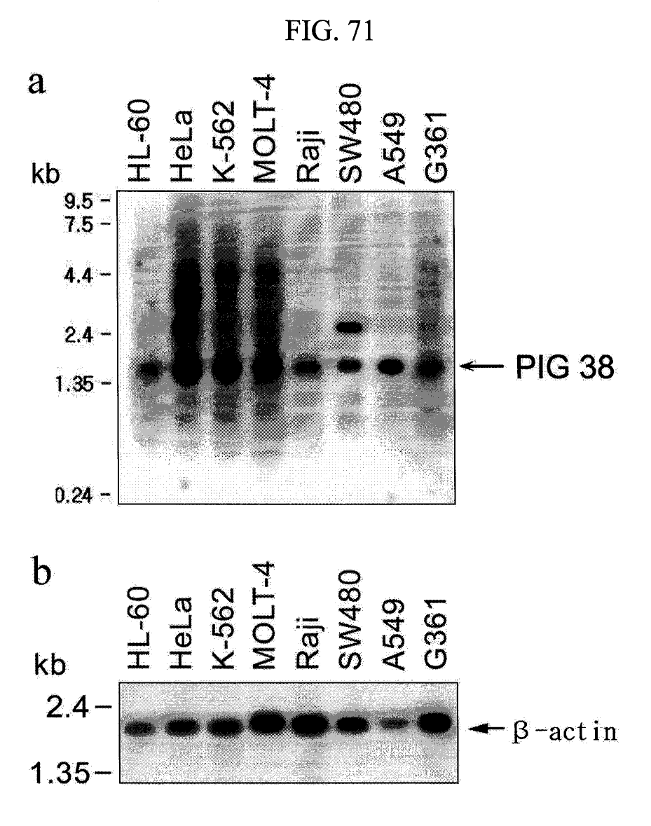

FIG. 50 shows a northern blotting result to determine whether or not the PIG38 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 50 shows the northern blotting result indicating whether or not β-actin mRNA is transcribed by hybridizing the same sample with β-actin probe. As shown in FIG. 50, it was revealed that a PIG38 mRNA transcript (approximately 1.5 kb) was not expressed or very rarely expressed in the various normal tissues such as the liver tissue. FIG. 71 shows a northern blotting result to determine whether or not the PIG38 protooncogene is expressed in the human cancer cell lines, for example HL-60, HeLa, K-562, MOLT-4, Raji, SW480, A549 and G361 (Clontech). A bottom of FIG. 71 shows the northern blotting result indicating whether or not β-actin mRNA is transcribed by hybridizing the same sample with β-actin probe. As shown in FIG. 71, it was revealed that a PIG38 mRNA transcript (approximately 1.5 kb) was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that another PIG38 mRNA transcripts having sizes of approximately 2.5 kb, 3 kb and 4.5 kb were simultaneously expressed in the cancer cell lines.

FIG. 31 shows a northern blotting result to determine whether or not the PIG43 protooncogene is expressed in the normal liver tissue, the liver cancer tissue and the liver cancer cell line (HepG2). As shown in FIG. 31, it was revealed that the PIG43 protooncogene was highly expressed in the liver cancer tissue and the liver cancer cell line HepG2, but not expressed or rarely expressed in the normal tissues. A bottom of FIG. 31 shows the northern blotting result indicating whether or not β-actin mRNA is transcribed by hybridizing the same sample with β-actin probe.

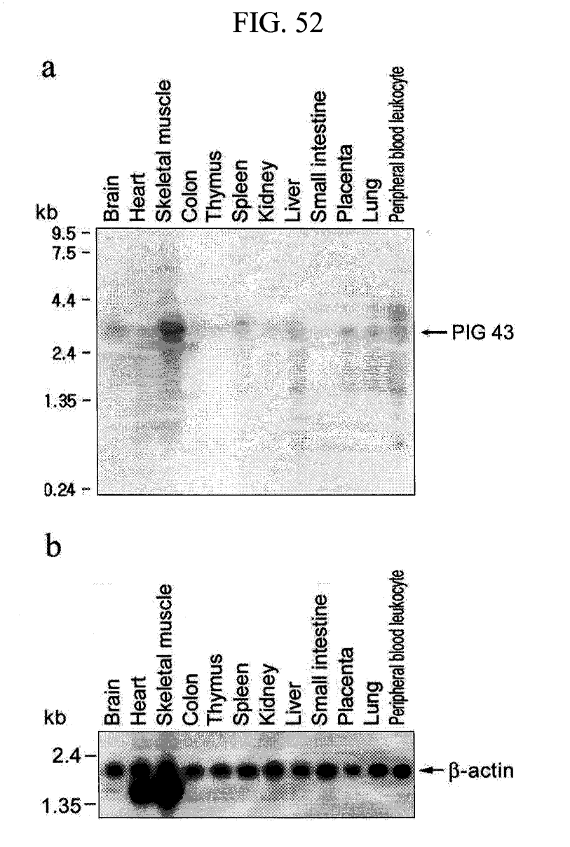

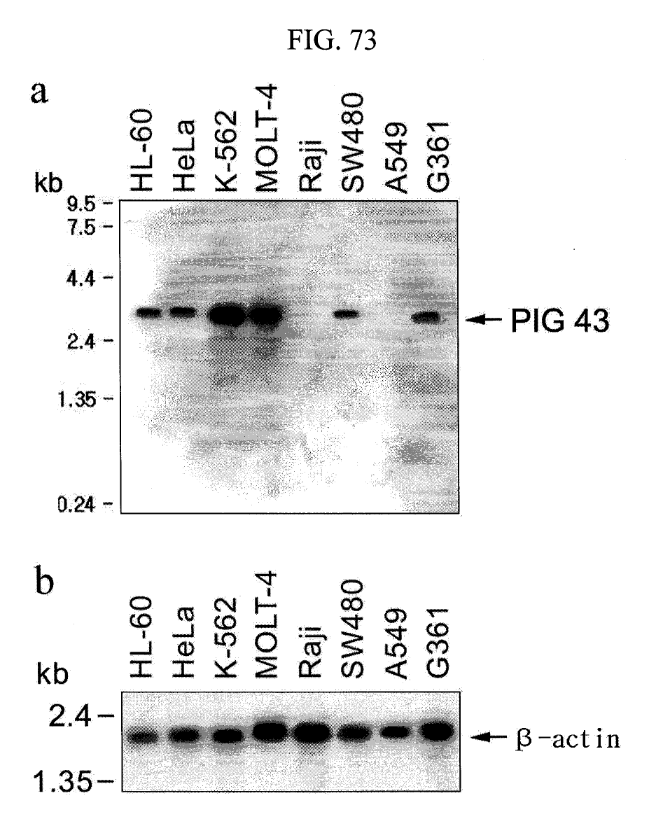

FIG. 52 shows a northern blotting result to determine whether or not the PIG43 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 52 shows the northern blotting result indicating whether or not β-actin mRNA is transcribed by hybridizing the same sample with β-actin probe. As shown in FIG. 52, it was revealed that a PIG43 mRNA transcript (approximately 3.0 kb) was not expressed or very rarely expressed in the various normal tissues such as the liver tissue. FIG. 73 shows a northern blotting result to determine whether or not the PIG43 protooncogene is expressed in the human cancer cell lines, for example HL-60, HeLa, K-562, MOLT-4, Raji, SW480, A549 and G361 (Clontech). A bottom of FIG. 73 shows the northern blotting result indicating whether or not β-actin mRNA is transcribed by hybridizing the same sample with β-actin probe. As shown in FIG. 73, it was revealed that a PIG43 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the colon cancer cell line SW480 and the skin cancer cell line G361, but not expressed in the Burkitt lymphoma cell line Raji and the lung cancer cell line A549.

FIG. 32 shows a northern blotting result to determine whether or not the PIG44 protooncogene is expressed in the normal liver tissue, the liver cancer tissue and the liver cancer cell line (HepG2). As shown in FIG. 32, it was revealed that the PIG44 protooncogene was highly expressed in the liver cancer tissue and the liver cancer cell line HepG2, but not expressed or rarely expressed in the normal tissues. A bottom of FIG. 32 shows the northern blotting result indicating whether or not β-actin mRNA is transcribed by hybridizing the same sample with β-actin probe.

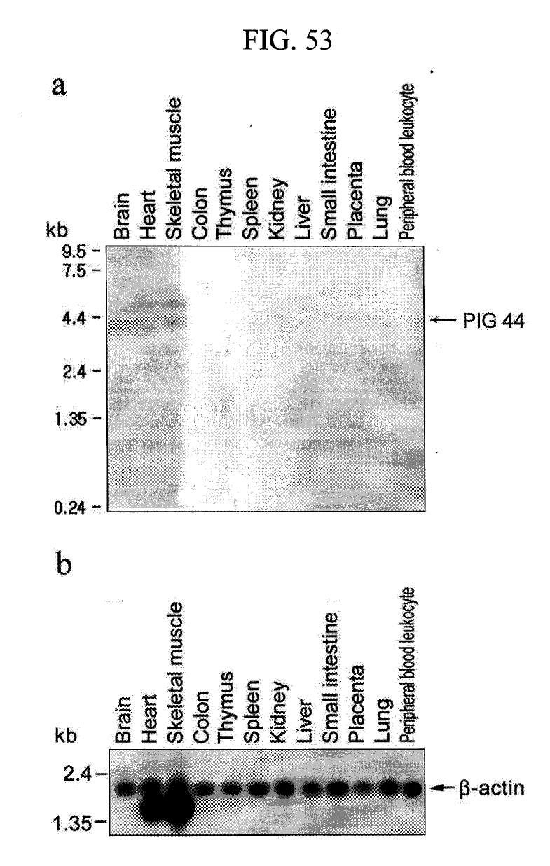

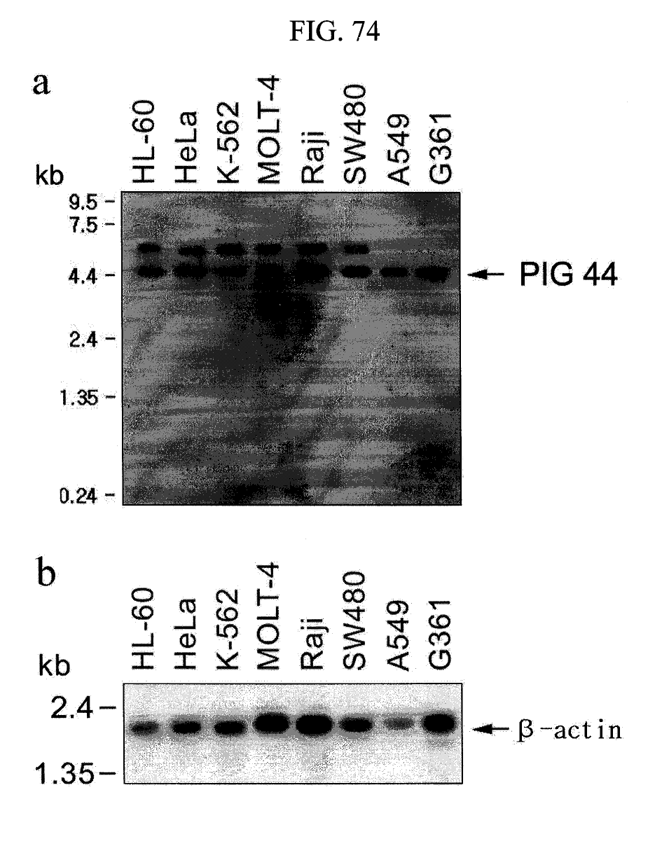

FIG. 53 shows a northern blotting result to determine whether or not the PIG44 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 53 shows the northern blotting result indicating whether or not β-actin mRNA is transcribed by hybridizing the same sample with β-actin probe. As shown in FIG. 53, it was revealed that a PIG44 mRNA transcript (approximately 4.5 kb) was not expressed or very rarely expressed in the various normal tissues such as the liver, but rarely expressed only in the normal heart and muscle. Also, it was revealed that a PIG44 mRNA transcript having a size of approximately 5.0 kb was very rarely expressed in the normal heart and muscle. FIG. 74 shows a northern blotting result to determine whether or not the PIG38 protooncogene is expressed in the human cancer cell lines, for example HL-60, HeLa, K-562, MOLT-4, Raji, SW480, A549 and G361 (Clontech). A bottom of FIG. 74 shows the northern blotting result indicating whether or not β-actin mRNA is transcribed by hybridizing the same sample with β-actin probe. As shown in FIG. 74, it was revealed that a PIG44 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that a PIG44 mRNA transcript having a size of approximately 5.0 kb was expressed at the same time.

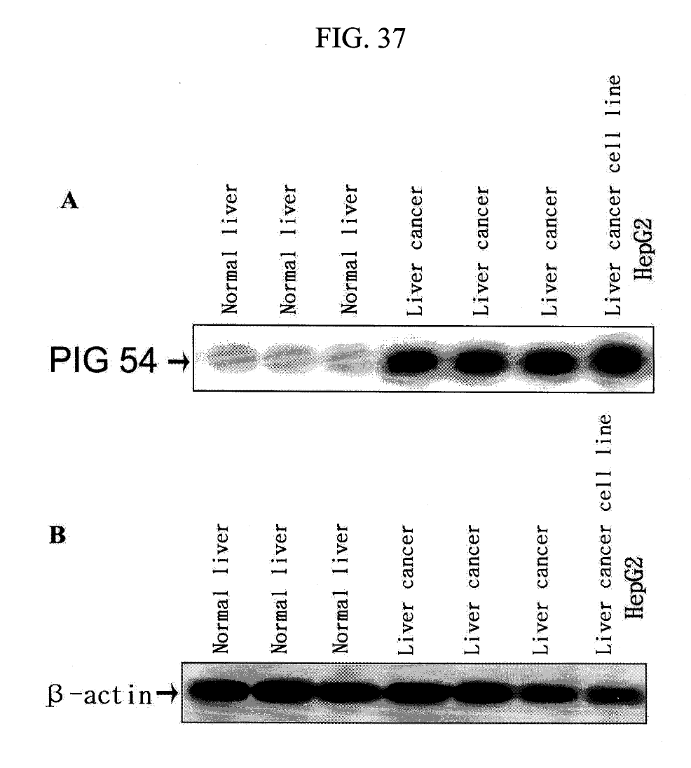

FIG. 37 shows a northern blotting result to determine whether or not the PIG54 protooncogene is expressed in the normal liver tissue, the liver cancer tissue and the liver cancer cell line (HepG2). As shown in FIG. 37, it was revealed that the PIG38 protooncogene was highly expressed in the liver cancer tissue and the liver cancer cell line HepG2, but not expressed or rarely expressed in the normal tissues. A bottom of FIG. 37 shows the northern blotting result indicating whether or not β-actin mRNA is transcribed by hybridizing the same sample with β-actin probe.

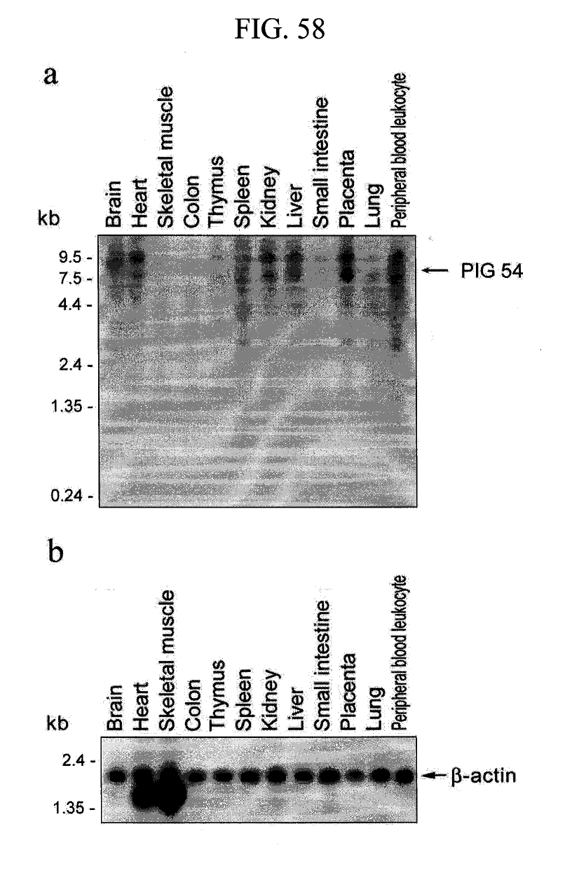

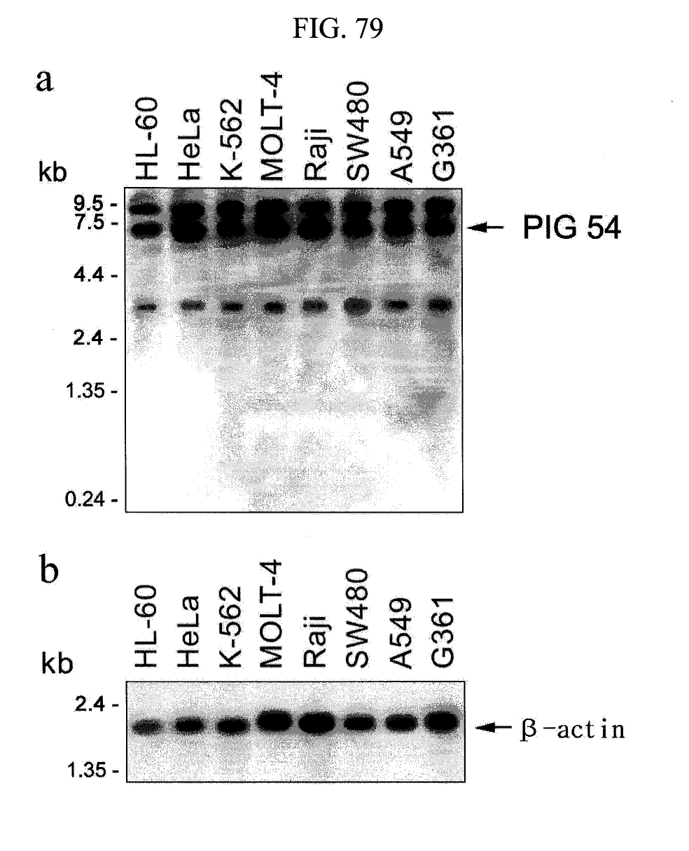

FIG. 58 shows a northern blotting result to determine whether or not the PIG54 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 58 shows the northern blotting result indicating whether or not β-actin mRNA is transcribed by hybridizing the same sample with β-actin probe. As shown in FIG. 58, it was revealed that a PIG54 mRNA transcript (approximately 7.0 kb) was not expressed or very rarely expressed in the various normal tissues such as the liver tissue. Also, it was revealed that a PIG54 mRNA transcript having a size of approximately 9.0 kb was very rarely expressed at the same time. FIG. 79 shows a northern blotting result to determine whether or not the PIG38 protooncogene is expressed in the human cancer cell lines, for example HL-60, HeLa, K-562, MOLT-4, Raji, SW480, A549 and G361 (Clontech). A bottom of FIG. 79 shows the northern blotting result indicating whether or not β-actin mRNA is transcribed by hybridizing the same sample with β-actin probe. As shown in FIG. 79, it was revealed that a PIG54 mRNA transcript was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that PIG54 mRNA transcripts having sizes of approximately 9.0 kb and 3.0 kb were highly expressed at the same time.

FIG. 41 shows a northern blotting result to determine whether or not the GIG18 protooncogene is expressed in the normal liver tissue, the liver cancer tissue and the liver cancer cell line (HepG2). As shown in FIG. 41, it was revealed that the GIG18 protooncogene was highly expressed in the liver cancer tissue and the liver cancer cell line HepG2, but rarely expressed in the normal tissues. A bottom of FIG. 41 shows the northern blotting result indicating whether or not β-actin mRNA is transcribed by hybridizing the same sample with β-actin probe.

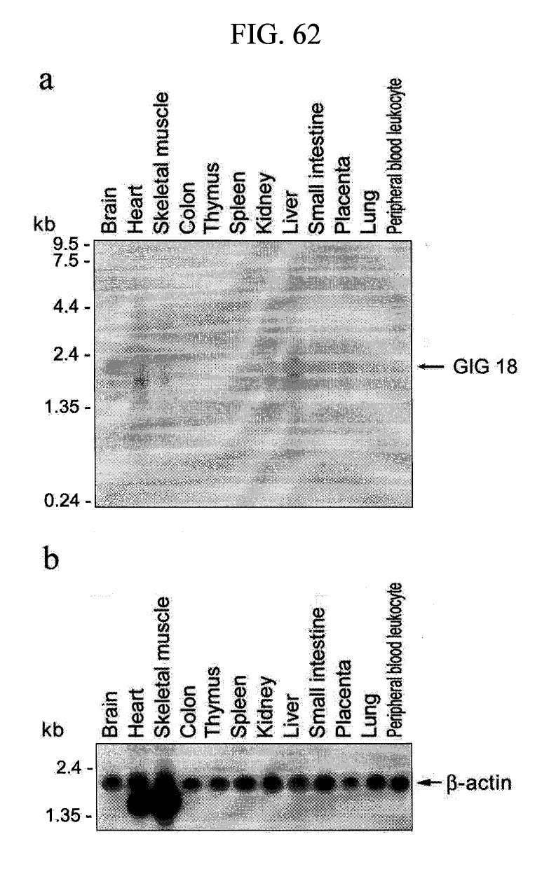

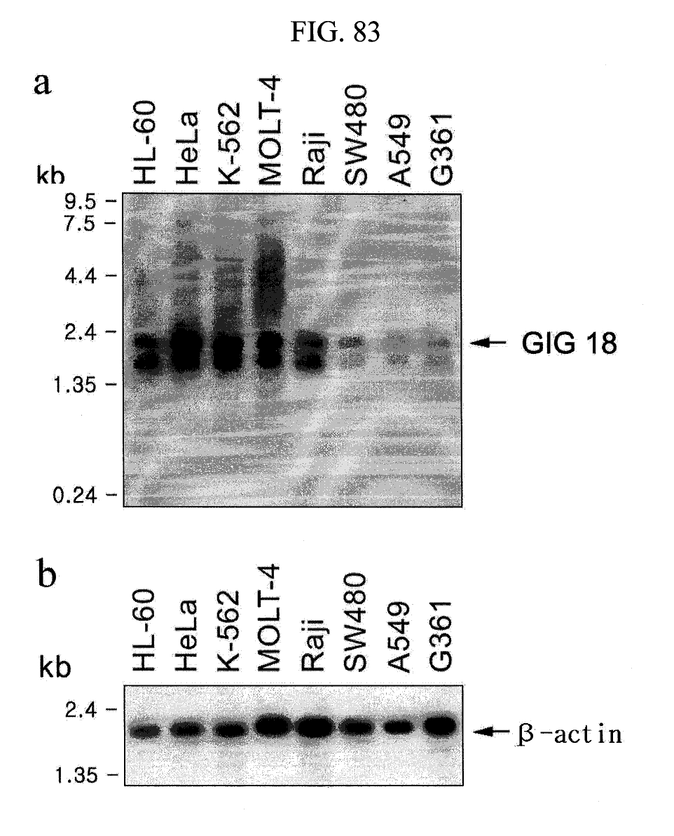

FIG. 62 shows a northern blotting result to determine whether or not the GIG18 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 62 shows the northern blotting result indicating whether or not β-actin mRNA is transcribed by hybridizing the same sample with β-actin probe. As shown in FIG. 62, it was revealed that a GIG18 mRNA transcript (approximately 2.2 kb) was not expressed or very rarely expressed in the various normal tissues such as the liver tissue. Also, it was revealed that another GIG18 mRNA transcript having a size of approximately 2.0 kb was rarely expressed at the same time. FIG. 83 shows a northern blotting result to determine whether or not the GIG18 protooncogene is expressed in the human cancer cell lines, for example HL-60, HeLa, K-562, MOLT-4, Raji, SW480, A549 and G361 (Clontech). A bottom of FIG. 83 shows the northern blotting result indicating whether or not β-actin mRNA is transcribed by hybridizing the same sample with β-actin probe. As shown in FIG. 83, it was revealed that a GIG18 mRNA transcript (approximately 2.2 kb) was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that another GIG18 mRNA transcript having a size of approximately 2.0 kb was highly expressed in the cancer cell lines at the same time.

7-4. PIG40

The total RNA samples were extracted from the normal peripheral blood tissue, the leukemia tissue and the K-562 cell in the same manner as in Example 1-4.

In order to determine an expression level of the PIG40 gene, 20 μg of each of the total denatured RNA samples extracted from the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed full-length PIG40 cDNA probe prepared using the Rediprime II random prime labelling system ((Amersham, United Kingdom). The northern blotting analysis was repeated twice, and then the resultant blots were quantitified with the densitometer and normalized with the β-actin.

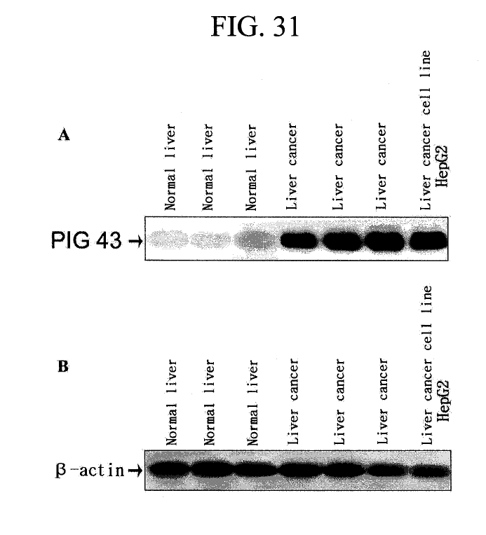

FIG. 30 shows a northern blotting result to determine whether or not the PIG40 protooncogene is expressed in the normal peripheral blood tissue, the leukemia tissue and the K-562 cell. As shown in FIG. 30, it was revealed that an expression level of the PIG40 protooncogene was increased, that is, a dominant PIG40 mRNA transcript having a size of approximately 2.5 kb was overexpressed in the leukemia tissue and the K-562 cell line. In FIG. 30, a lane “Normal” represents the normal peripheral blood tissue, a lane “Cancer” represents the leukemia tissue, and a line “K562” represents the leukemia cell line. A bottom of FIG. 30 shows the northern blotting result indicating whether or not β-actin mRNA is transcribed by hybridizing the same sample with β-actin probe.

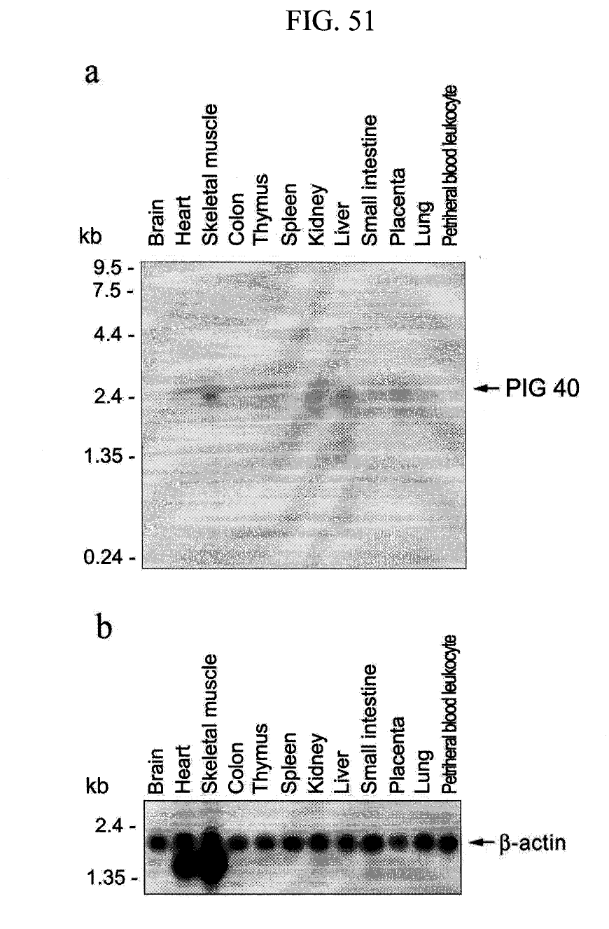

FIG. 51 shows a northern blotting result to determine whether or not the PIG40 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 51 shows the northern blotting result indicating whether or not β-actin mRNA is transcribed by hybridizing the same sample with β-actin probe. As shown in FIG. 51, it was revealed that a PIG40 mRNA transcript (the dominant PIG40 mRNA transcript having a size of approximately 2.5 kb) was very rarely expressed or not expressed in the various normal tissues such as the brain, the heart, the muscle, the large intestine, the thymus, the spleen, the kidney, the liver, the small intestine, the placenta, the lung and the peripheral blood leukocyte.

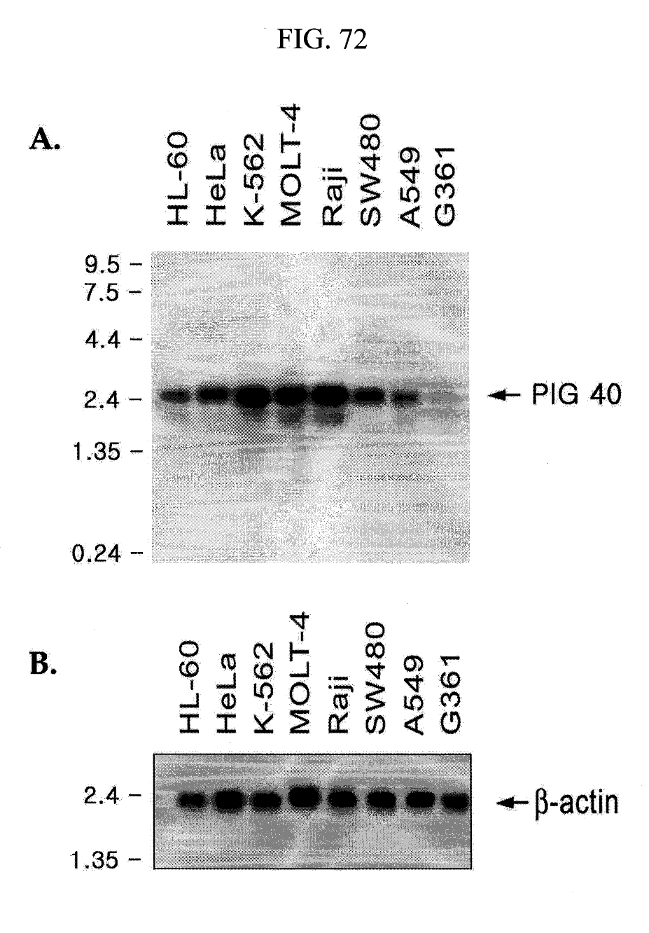

FIG. 72 shows a northern blotting result to determine whether or not the PIG40 protooncogene is expressed in the human cancer cell lines, for example HL-60, HeLa, K-562, MOLT-4, Raji, SW480, A549 and G361 (Clontech). A bottom of FIG. 72 shows the northern blotting result indicating whether or not β-actin mRNA is transcribed by hybridizing the same sample with β-actin probe. As shown in FIG. 72, it was revealed that a PIG40 mRNA transcript (the dominant PIG40 mRNA transcript having a size of approximately 2.5 kb) was highly expressed in the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that a PIG40 mRNA transcript having a size of approximately 2.0 kb was simultaneously expressed at an increased level in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

7-5. HLC-9 and MIG22

The total RNA samples were extracted from the normal lung tissue, the left lung cancer tissue, the metastatic lung cancer tissue metastasized from the left lung to the right lung, and the lung cancer cell lines A549, NCI-H2009 (American Type Culture Collection; ATCC Number CRL-5911) and NCI-H441 (American Type Culture Collection; ATCC Number HTB-174) in the same manner as in Example 1.

In order to determine an expression level of each of the HLC9 or MIG22 genes, 20 μg of each of the total denatured RNA samples extracted from the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed partical L738 or L690 cDNA probe prepared using the Rediprime II random prime labelling system ((Amersham, United Kingdom). The northern blotting analysis was repeated twice, and then the resultant blots were quantitified with the densitometer and normalized with the β-actin.

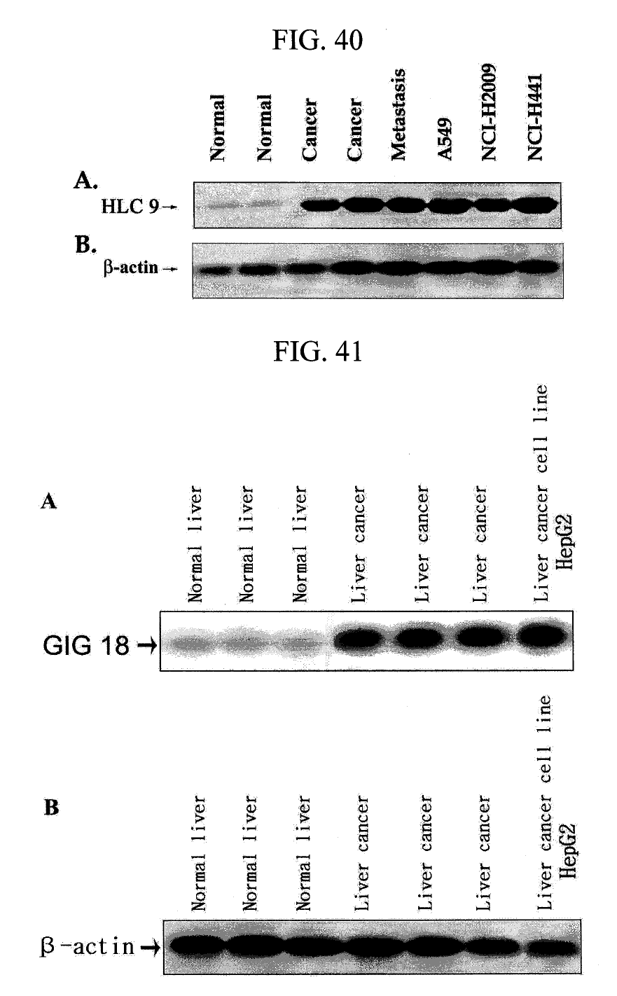

FIG. 40 shows a northern blotting result to determine whether or not the HLC9 protooncogene is expressed in the normal lung tissue, the lung cancer tissue, the metastatic lung cancer tissue and the lung cancer cell lines (A549, NCI-H2009 and NCI-H441). As shown in FIG. 40, it was revealed that the HLC9 protooncogene was highly expressed in the lung cancer tissue, the metastatic lung cancer tissue and the lung cancer cell lines A549, NCI-H2009 and NCI-H441, but rarely expressed or not expressed in the normal lung tissues. In FIG. 40, a lane “Normal” represents the normal lung tissue, a lane “Cancer” represents the lung cancer tissue, a lane “Metastasis” represents the metastatic lung cancer tissue, and lines “A549”, “NCI-H2009” and “NCI-H441” represent the lung cancer cell line. A bottom of FIG. 40 shows the northern blotting result indicating whether or not β-actin mRNA is transcribed by hybridizing the same sample with β-actin probe.

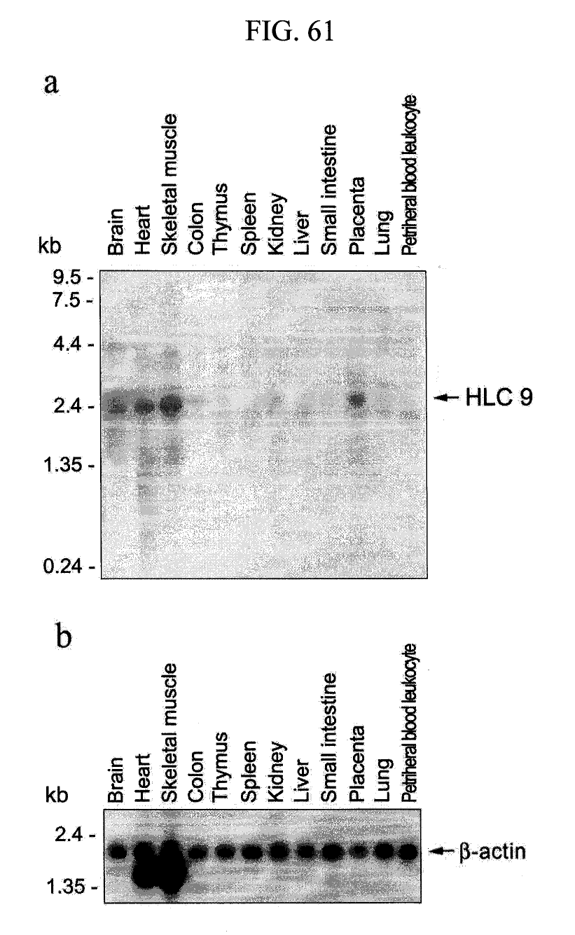

FIG. 61 shows a northern blotting result to determine whether or not the HLC9 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 61 shows the northern blotting result indicating whether or not β-actin mRNA is transcribed by hybridizing the same sample with β-actin probe. As shown in FIG. 61, it was revealed that an HLC9 mRNA transcript (approximately 2.5 kb) was expressed in the normal tissues such as the muscle, the heart and the placenta, and very expressed or not expressed in the other normal tissues. Also, it was revealed that another HLC9 mRNA transcript having a size of approximately 4.4 kb was very rarely expressed or not expressed in the normal tissue at the same time.

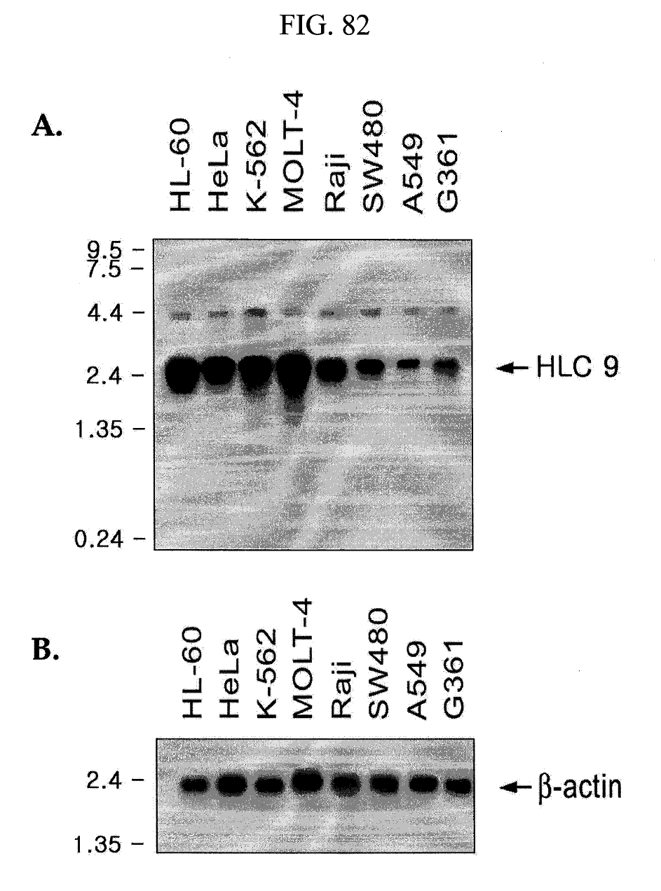

FIG. 82 shows a northern blotting result to determine whether or not the HLC9 protooncogene is expressed in the human cancer cell lines, for example HL-60, HeLa, K-562, MOLT-4, Raji, SW480, A549 and G361 (Clontech). A bottom of FIG. 82 shows the northern blotting result indicating whether or not β-actin mRNA is transcribed by hybridizing the same sample with β-actin probe. As shown in FIG. 82, it was revealed that an HLC9 mRNA transcript (approximately 1.5 kb) was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, it was revealed that another HLC9 mRNA transcript having a size of approximately 4.4 kb was highly expressed in the cancer cell lines at the same time.

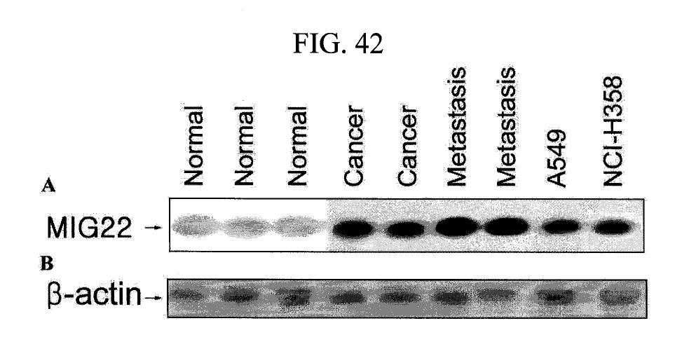

FIG. 42 shows a northern blotting result to determine whether or not the MIG22 protooncogene is expressed in the normal lung tissue, the lung cancer tissue, the metastatic lung cancer tissue and the lung cancer cell lines (A549 and NCI-H358). As shown in FIG. 42, it was revealed that the MIG22 protooncogene was highly expressed in the lung cancer tissue, the metastatic lung cancer tissue and the lung cancer cell lines A549 and NCI-H358, but rarely expressed or not expressed in the normal lung tissues. In FIG. 42, a lane “Normal” represents the normal lung tissue, a lane “Cancer” represents the lung cancer tissue, a lane “Metastasis” represents the metastatic lung cancer tissue, and lines “A549” and “NCI-H358” represent the lung cancer cell line. A bottom of FIG. 42 shows the northern blotting result indicating whether or not β-actin mRNA is transcribed by hybridizing the same sample with β-actin probe.

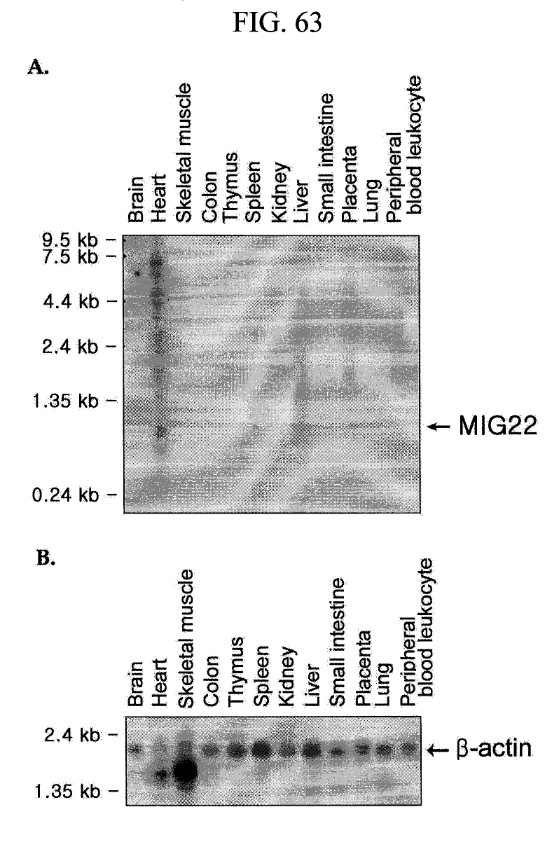

FIG. 63 shows a northern blotting result to determine whether or not the MIG22 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 63 shows the northern blotting result indicating whether or not β-actin mRNA is transcribed by hybridizing the same sample with β-actin probe. As shown in FIG. 63, it was revealed that an MIG22 mRNA transcript (the transcript having a size of approximately 1.0 kb) was very rarely expressed or not expressed in the normal tissues.

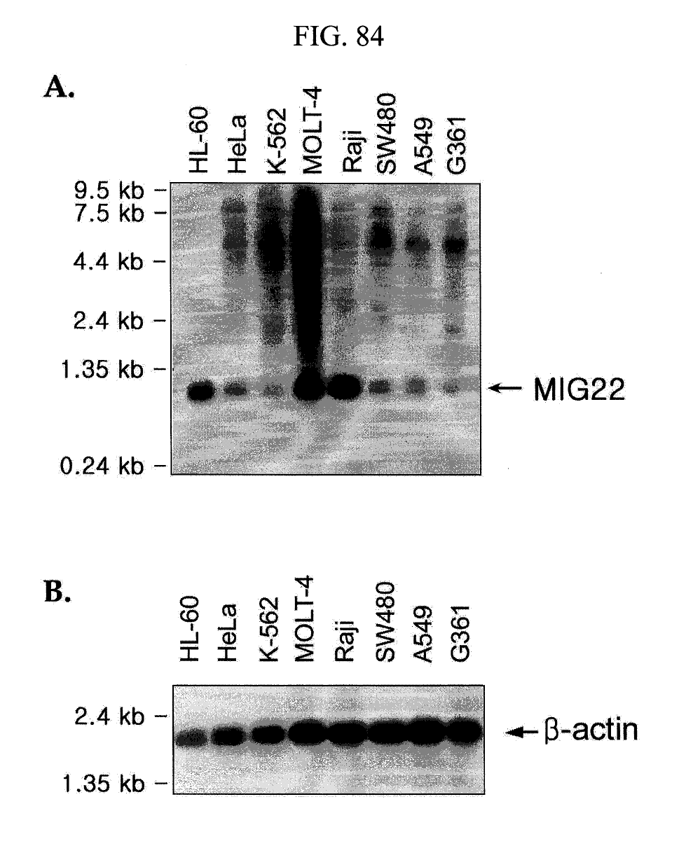

FIG. 84 shows a northern blotting result to determine whether or not the MIG22 protooncogene is expressed in the human cancer cell lines, for example HL-60, HeLa, K-562, MOLT-4, Raji, SW480, A549 and G361 (Clontech). A bottom of FIG. 84 shows the northern blotting result indicating whether or not β-actin mRNA is transcribed by hybridizing the same sample with β-actin probe. As shown in FIG. 84, it was revealed that MIG22 mRNA transcripts (the dominant transcript having a size of approximately 1.0 kb, and another transcripts having sizes of approximately 5.0 kb and 8.0 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

### Example 8

### Size Determination of Protein Expressed after Transforming E. coli with Protooncogene

Each of the PIG12 protooncogene of SEQ ID NO: 1; the PIG18 protooncogene of SEQ ID NO: 5; the PIG23 protooncogene of SEQ ID NO: 9; the PIG27 protooncogene of SEQ ID NO: 13; the PIG28 protooncogene of SEQ ID NO: 17; the PIG30 protooncogene of SEQ ID NO: 21; the PIG31 protooncogene of SEQ ID NO: 25; the PIG38 protooncogene of SEQ ID NO: 29; the PIG40 protooncogene of SEQ ID NO: 33; the PIG43 protooncogene of SEQ ID NO: 37; the PIG44 protooncogene of SEQ ID NO: 41; the PIG46 protooncogene of SEQ ID NO: 45; the PIG47 protooncogene of SEQ ID NO: 49; the PIG48 protooncogene of SEQ ID NO: 53; the PIG50 protooncogene of SEQ ID NO: 57; the PIG54 protooncogene of SEQ ID NO: 61; the PIG55 protooncogene of SEQ ID NO: 65; the GIG9 protooncogene of SEQ ID NO: 69; the HLC-9 protooncogene of SEQ ID NO: 73; the GIG18 protooncogene of SEQ ID NO: 77; and the MIG22 protooncogene of SEQ ID NO: 81 was inserted into a multi-cloning site of the pBAD/thio-TOPO vector (Invitrogen), and then E. coli was transformed with each of the resultant expression vectors. Each of the transformed E. coli strains was incubated in LB broth while shaking, and then each of the resultant cultures was diluted at a ratio of 1/100 and incubated for 3 hours again. 1 mM isopropyl beta-D-thiogalacto-pyranoside (IPTG, Sigma) was added thereto to facilitate production of their proteins. The E. coli cells were sonicated in the culture media before/after IPTG induction, and then the sonicated homogenates were subject to 12% sodium dodecyl sulfate-polyacrylamide gel electrophoresis (SDS-PAGE). The SDS-PAGE was conducted after the protein samples were obtained from the culture media according to the method described in the cited reference (Sambrook, J. et al., Molecular Cloning: A Laboratory manual, New York: Cold Spring Harbor Laboratory (1989)).

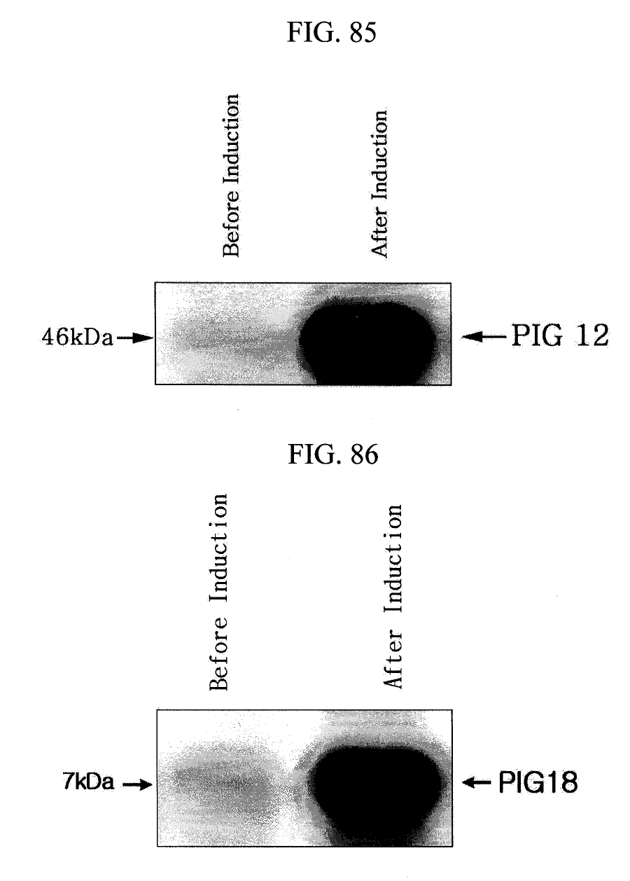

FIG. 85 is a diagram showing an SDS-PAGE analysis result on the PIG12 protein. In FIG. 85, a lane 1 represents a protein sample before IPTG induction, and a line 2 represents a protein sample after expression of the PIG12 gene is induced by IPTG. As shown in FIG. 85, the expressed PIG12 protein has a molecular weight of approximately 46 kDa, which corresponds to the molecular weight derived from its DNA sequence.

FIG. 86 shows a SDS-PAGE result to determine an expression pattern of proteins in the E. coli Top 10 strain transformed with the pBAD/thio-Topo/PIG 18 vector, wherein a band of a fusion protein having a molecular weight of approximately 22 kDa was clearly observed after L-arabinose induction. The 15-kDa fusion protein includes the HT-thioredoxin protein having a molecular weight of approximately 15 kDa and the PIG18 protein having a molecular weight of approximately 7 kDa, each protein being inserted into the pBAD/thio-Topo/PIG18 vector.

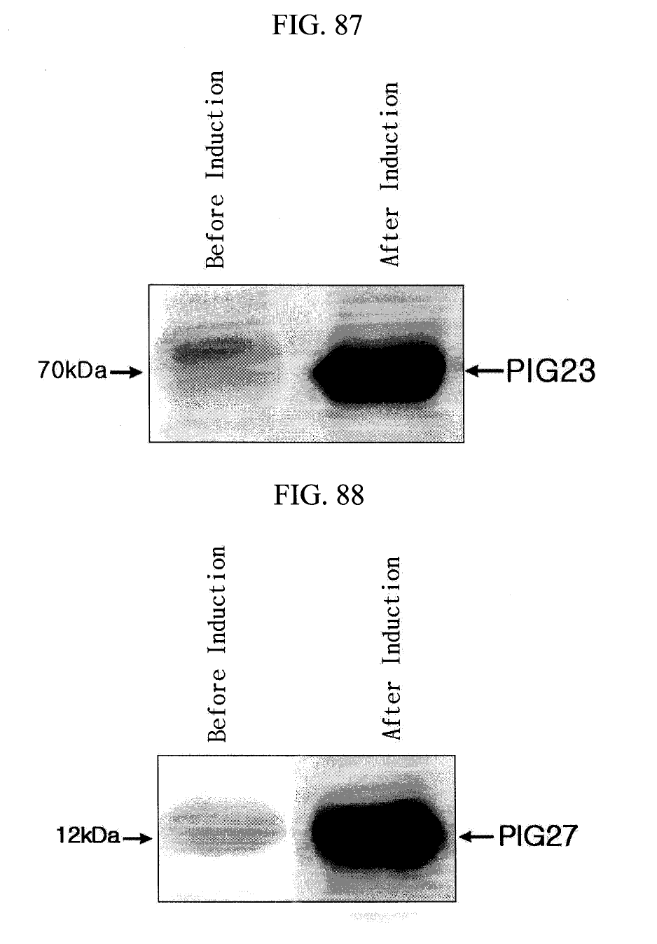

FIG. 87 shows a SDS-PAGE result to determine an expression pattern of proteins in the E. coli Top 10 strain transformed with the pBAD/thio-Topo/PIG28 vector, wherein a band of a fusion protein having a molecular weight of approximately 85 kDa was clearly observed after L-arabinose induction. The 85-kDa fusion protein includes the HT-thioredoxin protein having a molecular weight of approximately 15 kDa and the PIG23 protein having a molecular weight of approximately 70 kDa, each protein being inserted into the pBAD/thio-Topo/PIG23 vector.

FIG. 88 shows a SDS-PAGE result to determine an expression pattern of proteins in the E. coli Top 10 strain transformed with the pBAD/thio-Topo/PIG27 vector, wherein a band of a fusion protein having a molecular weight of approximately 27 kDa was clearly observed after L-arabinose induction. The 27-kDa fusion protein includes the HT-thioredoxin protein having a molecular weight of approximately 15 kDa and the PIG27 protein having a molecular weight of approximately 12 kDa, each protein being inserted into the pBAD/thio-Topo/PIG27 vector.

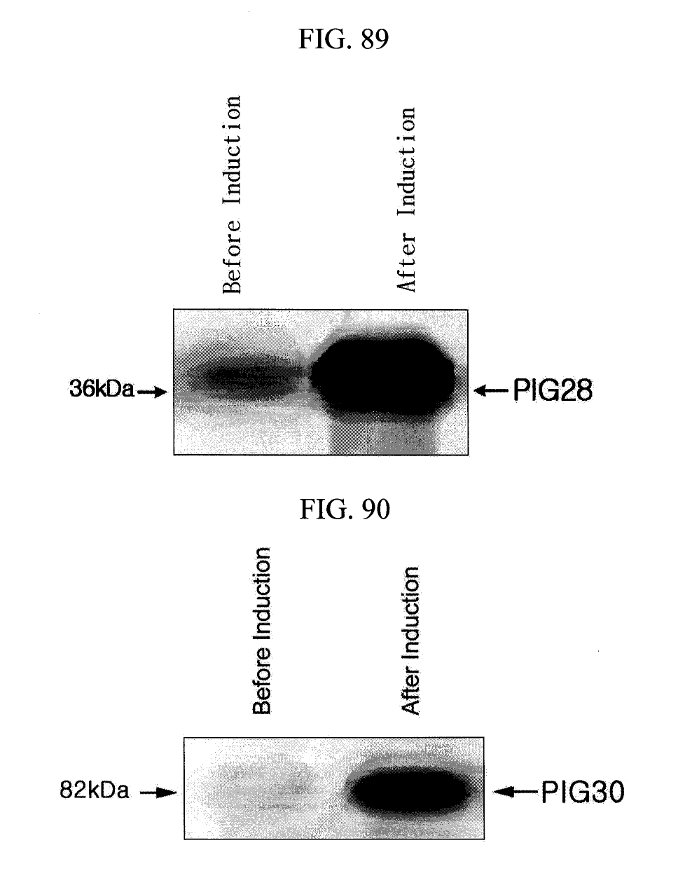

FIG. 89 shows a SDS-PAGE result to determine an expression pattern of proteins in the E. coli Top 10 strain transformed with the pBAD/thio-Topo/PIG28 vector, wherein a band of a fusion protein having a molecular weight of approximately 51 kDa was clearly observed after L-arabinose induction. The 51-kDa fusion protein includes the HT-thioredoxin protein having a molecular weight of approximately 15 kDa and the PIG28 protein having a molecular weight of approximately 36 kDa, each protein being inserted into the pBAD/thio-Topo/PIG28 vector.

FIG. 90 is a diagram showing an SDS-PAGE analysis result on the PIG30 protein. In FIG. 90, a lane 1 represents a protein sample before IPTG induction, and a line 2 represents a protein sample after expression of the PIG30 gene is induced by IPTG. As shown in FIG. 90, the expressed PIG30 protein has a molecular weight of approximately 82 kDa, which corresponds to the molecular weight derived from its DNA sequence.

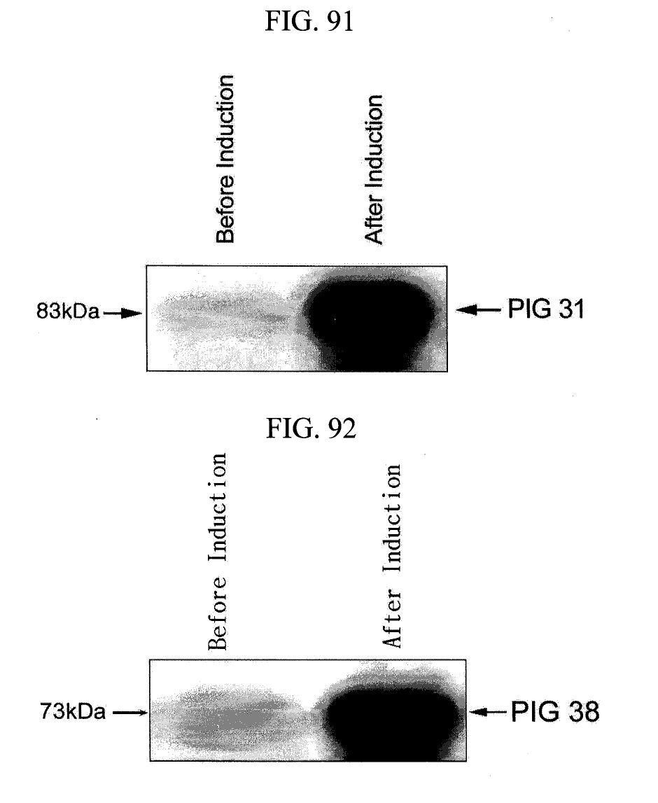

FIG. 91 is a diagram showing an SDS-PAGE analysis result on the PIG31 protein. In FIG. 91, a lane 1 represents a protein sample before IPTG induction, and a line 2 represents a protein sample after expression of the PIG31 gene is induced by IPTG. As shown in FIG. 91, the expressed PIG31 protein has a molecular weight of approximately 83 kDa, which corresponds to the molecular weight derived from its DNA sequence.

FIG. 92 shows a SDS-PAGE result to determine an expression pattern of proteins in the E. coli Top 10 strain transformed with the pBAD/thio-Topo/PIG38 vector, wherein a band of a fusion protein having a molecular weight of approximately 88 kDa was clearly observed after L-arabinose induction. The 88-kDa fusion protein includes the HT-thioredoxin protein having a molecular weight of approximately 15 kDa and the PIG38 protein having a molecular weight of approximately 73 kDa, each protein being inserted into the pBAD/thio-Topo/PIG38 vector.

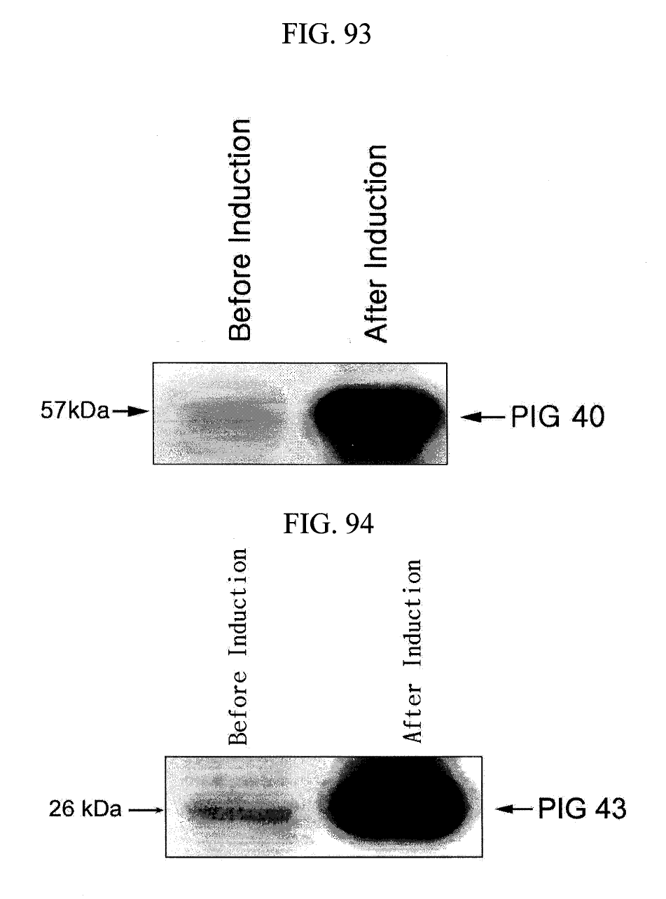

FIG. 93 shows a SDS-PAGE result to determine an expression pattern of proteins in the E. coli Top 10 strain transformed with the pBAD/thio-Topo/PIG40 vector, wherein a band of a fusion protein having a molecular weight of approximately 72 kDa was clearly observed after L-arabinose induction. The 72-kDa fusion protein includes the HT-thioredoxin protein having a molecular weight of approximately 15 kDa and the PIG40 protein having a molecular weight of approximately 57 kDa, each protein being inserted into the pBAD/thio-Topo/PIG40 vector.

FIG. 94 shows a SDS-PAGE result to determine an expression pattern of proteins in the E. coli Top 10 strain transformed with the pBAD/thio-Topo/PIG43 vector, wherein a band of a fusion protein having a molecular weight of approximately 41 kDa was clearly observed after L-arabinose induction. The 41-kDa fusion protein includes the HT-thioredoxin protein having a molecular weight of approximately 15 kDa and the PIG43 protein having a molecular weight of approximately 26 kDa, each protein being inserted into the pBAD/thio-Topo/PIG43 vector.

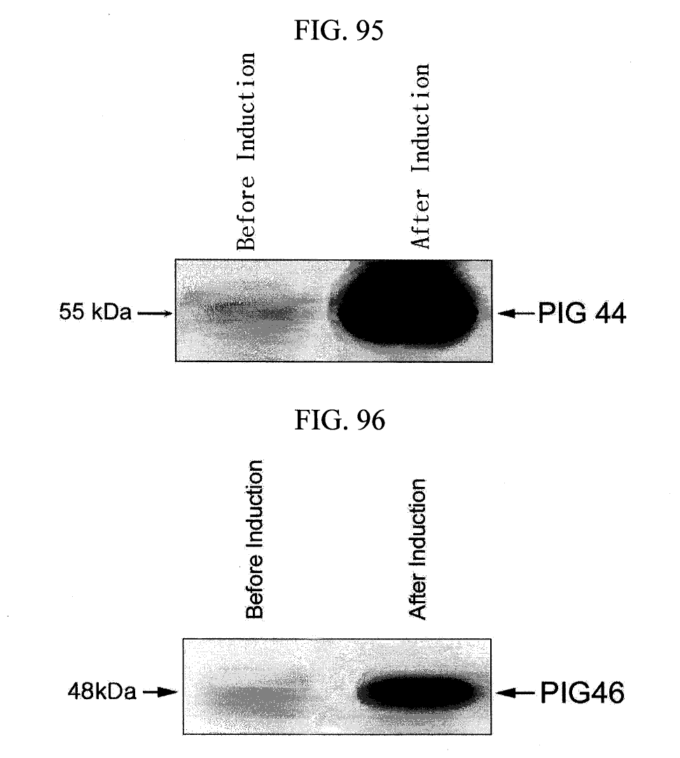

FIG. 95 shows a SDS-PAGE result to determine an expression pattern of proteins in the E. coli Top 10 strain transformed with the pBAD/thio-Topo/PIG44 vector, wherein a band of a fusion protein having a molecular weight of approximately 70 kDa was clearly observed after L-arabinose induction. The 70-kDa fusion protein includes the HT-thioredoxin protein having a molecular weight of approximately 15 kDa and the PIG44 protein having a molecular weight of approximately 55 kDa, each protein being inserted into the pBAD/thio-Topo/PIG44 vector.

FIG. 96 is a diagram showing an SDS-PAGE analysis result on the PIG46 protein. In FIG. 96, a lane 1 represents a protein sample before IPTG induction, and a line 2 represents a protein sample after expression of the PIG46 gene is induced by IPTG. As shown in FIG. 96, the expressed PIG46 protein has a molecular weight of approximately 48 kDa, which corresponds to the molecular weight derived from its DNA sequence.

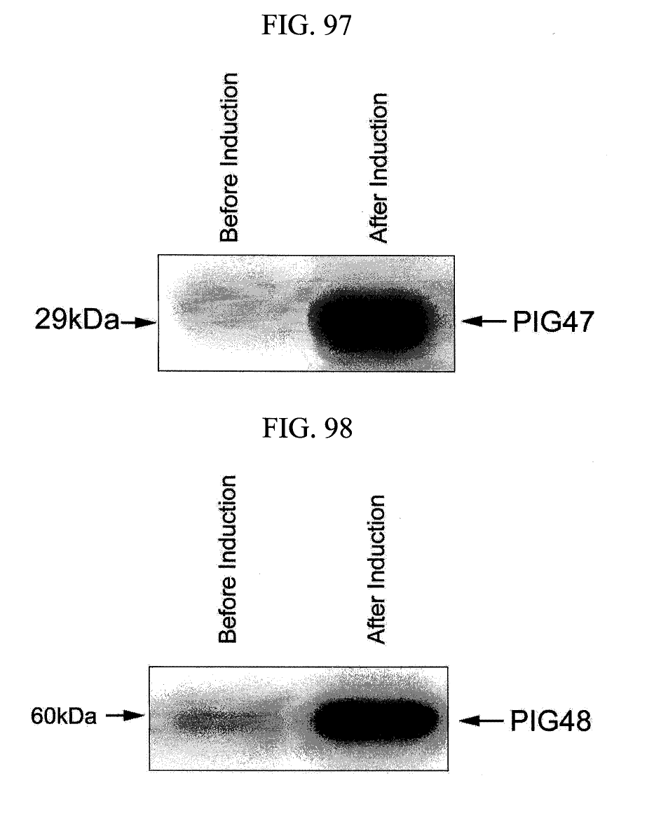

FIG. 97 is a diagram showing an SDS-PAGE analysis result on the PIG47 protein. In FIG. 97, a lane 1 represents a protein sample before IPTG induction, and a line 2 represents a protein sample after expression of the PIG47 gene is induced by IPTG. As shown in FIG. 97, the expressed PIG47 protein has a molecular weight of approximately 29 kDa, which corresponds to the molecular weight derived from its DNA sequence.

FIG. 98 is a diagram showing an SDS-PAGE analysis result on the PIG48 protein. In FIG. 98, a lane 1 represents a protein sample before IPTG induction, and a line 2 represents a protein sample after expression of the PIG48 gene is induced by IPTG. As shown in FIG. 98, the expressed PIG48 protein has a molecular weight of approximately 60 kDa, which corresponds to the molecular weight derived from its DNA sequence.

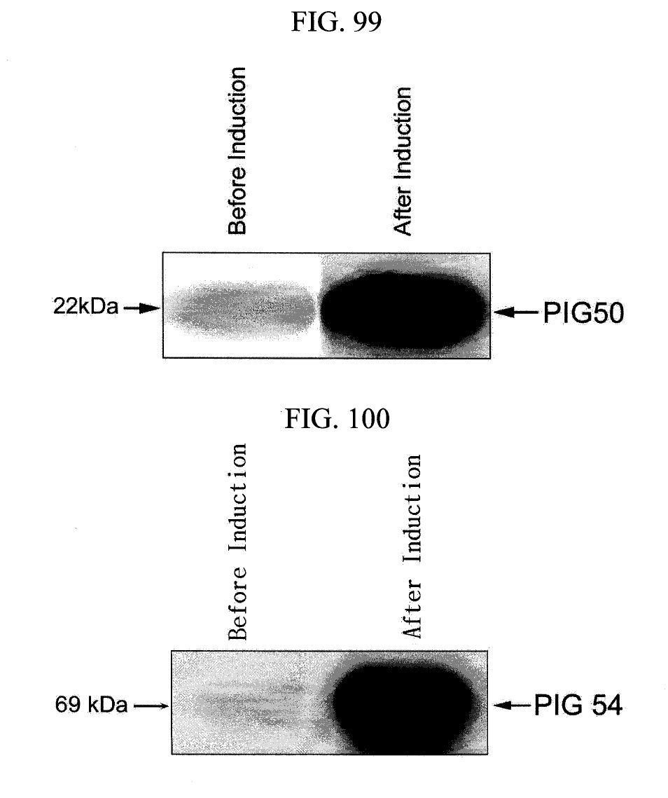

FIG. 99 is a diagram showing an SDS-PAGE analysis result on the PIG50 protein. In FIG. 99, a lane 1 represents a protein sample before IPTG induction, and a line 2 represents a protein sample after expression of the PIG50 gene is induced by IPTG. As shown in FIG. 99, the expressed PIG50 protein has a molecular weight of approximately 22 kDa, which corresponds to the molecular weight derived from its DNA sequence.

FIG. 100 shows a SDS-PAGE result to determine an expression pattern of proteins in the E. coli Top 10 strain transformed with the pBAD/thio-Topo/PIG54 vector, wherein a band of a fusion protein having a molecular weight of approximately 84 kDa was clearly observed after L-arabinose induction. The 84-kDa fusion protein includes the HT-thioredoxin protein having a molecular weight of approximately 15 kDa and the PIG54 protein having a molecular weight of approximately 69 kDa, each protein being inserted into the pBAD/thio-Topo/PIG54 vector.

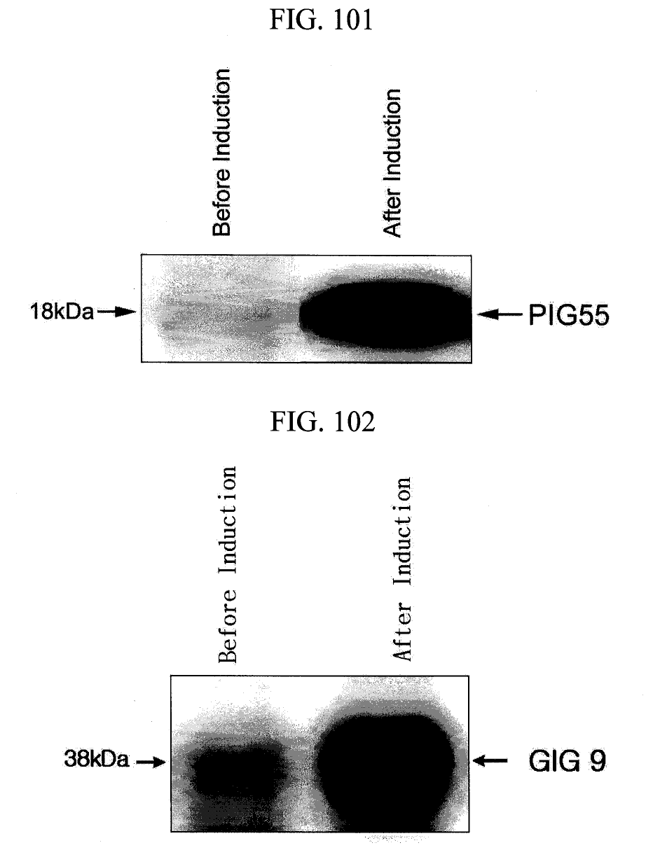

FIG. 101 is a diagram showing an SDS-PAGE analysis result on the PIG55 protein. In FIG. 101, a lane 1 represents a protein sample before IPTG induction, and a line 2 represents a protein sample after expression of the PIG55 gene is induced by IPTG. As shown in FIG. 101, the expressed PIG55 protein has a molecular weight of approximately 18 kDa, which corresponds to the molecular weight derived from its DNA sequence.

FIG. 102 shows a SDS-PAGE result to determine an expression pattern of proteins in the E. coli Top 10 strain transformed with the pBAD/thio-Topo/GIG9 vector, wherein a band of a fusion protein having a molecular weight of approximately 53 kDa was clearly observed after L-arabinose induction. The 53-kDa fusion protein includes the HT-thioredoxin protein having a molecular weight of approximately 15 kDa and the GIG9 protein having a molecular weight of approximately 38 kDa, each protein being inserted into the pBAD/thio-Topo/GIG9 vector.

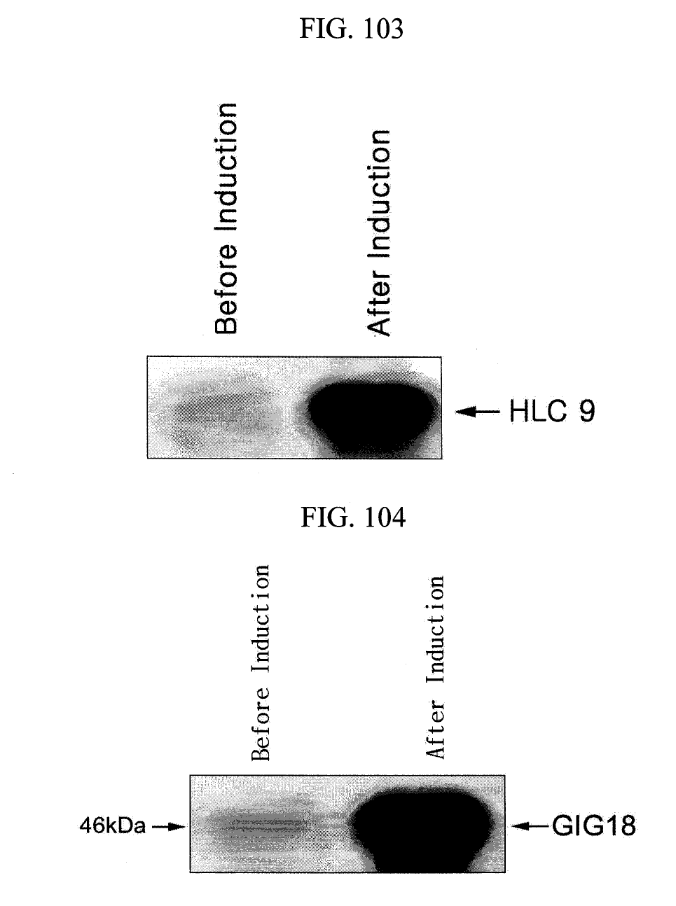

FIG. 103 shows a SDS-PAGE result to determine an expression pattern of proteins in the E. coli Top 10 strain transformed with the pBAD/thio-Topo/HLC9 vector, wherein a band of a fusion protein having a molecular weight of approximately 66 kDa was clearly observed after L-arabinose induction. The 66-kDa fusion protein includes the HT-thioredoxin protein having a molecular weight of approximately 15 kDa and the HLC9 protein having a molecular weight of approximately 51 kDa, each protein being inserted into the pBAD/thio-Topo/HLC9 vector.

FIG. 104 shows a SDS-PAGE result to determine an expression pattern of proteins in the E. coli Top 10 strain transformed with the pBAD/thio-Topo/GIG18 vector, wherein a band of a fusion protein having a molecular weight of approximately 61 kDa was clearly observed after L-arabinose induction. The 61-kDa fusion protein includes the HT-thioredoxin protein having a molecular weight of approximately 15 kDa and the GIG18 protein having a molecular weight of approximately 46 kDa, each protein being inserted into the pBAD/thio-Topo/GIG18 vector.

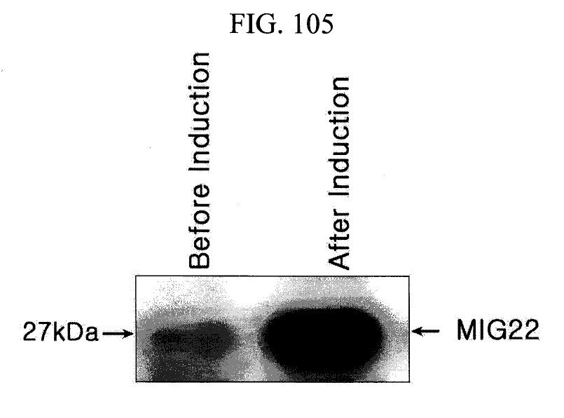

FIG. 105 shows a SDS-PAGE result to determine an expression pattern of proteins in the E. coli Top 10 strain transformed with the pBAD/thio-Topo/MIG22 vector, wherein a band of a fusion protein having a molecular weight of approximately 42 kDa was clearly observed after L-arabinose induction. The 42-kDa fusion protein includes the HT-thioredoxin protein having a molecular weight of approximately 15 kDa and the MIG22 protein having a molecular weight of approximately 27 kDa, each protein being inserted into the pBAD/thio-Topo/MIG22 vector.

### INDUSTRIAL APPLICABILITY

The protooncogenes of the present invention may be effectively used for diagnosing various cancers including breast cancer, leukemia, uterine cancer, lung cancer, malignant lymphoma, etc.

What is claimed is:

1. A human protooncoprotein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; SEQ ID NO: 58; SEQ ID NO: 62; SEQ ID NO: 66; SEQ ID NO: 70; SEQ ID NO: 74; SEQ ID NO: 78; and SEQ ID NO: 82.

2. A human protooncogene having a DNA sequence selected from the group consisting of a DNA sequence corresponding to nucleotide sequence positions from 68 to 1,252 of SEQ ID NO: 1; a DNA sequence corresponding to nucleotide sequence positions from 875 to 1,063 of SEQ ID NO: 5; a DNA sequence corresponding to nucleotide sequence positions from 25 to 1,953 of SEQ ID NO: 9; a DNA sequence corresponding to nucleotide sequence positions from 20 to 337 of SEQ ID NO: 13; a DNA sequence corresponding to nucleotide sequence positions from 33 to 998 of SEQ ID NO: 17; a DNA sequence corresponding to nucleotide sequence positions from position 6 to 2,150 of SEQ ID NO: 21; a DNA sequence corresponding to nucleotide sequences 37 to 2,232 of SEQ ID NO: 25; a DNA sequence corresponding to nucleotide sequence positions from 25 to 1,956 of SEQ ID NO: 29; a DNA sequence corresponding to nucleotide sequence positions from 36 to 1,541 of SEQ ID NO: 33; a DNA sequence corresponding to nucleotide sequence positions from 57 to 758 of SEQ ID NO: 37; a DNA sequence corresponding to nucleotide sequence positions from 55 to 1,512 of SEQ ID NO: 41; a DNA sequence corresponding to nucleotide sequence positions from 5 to 1,297 of SEQ ID NO: 45; a DNA sequence corresponding to nucleotide sequence positions from 56 to 826 of SEQ ID NO: 49; a DNA sequence corresponding to nucleotide sequence positions from 57 to 1,694 of SEQ ID NO: 53; a DNA sequence corresponding to nucleotide sequence positions from 2 to 595 of SEQ ID NO: 57; a DNA sequence corresponding to nucleotide sequence positions from 38 to 1,840 of SEQ ID NO: 61; a DNA sequence corresponding to nucleotide sequence positions from 15 to 485 of SEQ ID NO: 65; a DNA sequence corresponding to nucleotide sequence positions from 1 to 1,008 of SEQ ID NO: 69; a DNA sequence corresponding to nucleotide sequence positions from 27 to 1,370 of SEQ ID NO: 73; a DNA sequence corresponding to nucleotide sequence positions from 3 to 1,244 of SEQ ID NO: 77; and a DNA sequence corresponding to nucleotide sequence positions from 15 to 734 of SEQ ID NO: 81, wherein the DNA sequences encode the protooncoproteins defined in claim 1, respectivley.

3. A kit for diagnosing cancer including the protooncoprotein defined in claim 1.

4. A kit for diagnosing cancer including the protooncogene as defined in claim 2.
